SEQUENCE LISTING (172 sequences)

SEQ ID NO: 1 - protein, 235 aa, Artificial Sequence
  in vitro translated mutant E. coli RS3 protein
MGQKVHPNGIRLGIVKPWNSTWFANTKEFADNLDSDFKVRQYLTKELAKASVSRIVIERPAKSIRVTIHTARPGIVIGKKGEDVEKLRKVVADIAGVPAQINIAEVRKPELDAKLVADSITSQLERRVMFRRAMKRAVQNAMRLGAKGIKVEVSGRLGGAEIARTEWYREGRVPLHTLRADIDYNTSEAHTTYGVIGVKVWIFKGEILGGMAAVEQPEKPAAQHSRKNSSVKVVN

SEQ ID NO: 2 - protein, 47 aa, Artificial Sequence
  in vitro translated mutant E. coli RS3 protein
DIDYNTSEAHTTYGVIGVKVWIFKGEILGGMAAVEQPEKPAAQHSRK

SEQ ID NO: 3 - protein, 43 aa, Artificial Sequence
  in vitro translated mutant E. coli RS3 protein
DIDYNTSEAHTTYGVIGVKVWIFKGEILGGMAAVEQPEKPAAQ

SEQ ID NO: 4 - protein, 47 aa, Artificial Sequence
  in vitro translated E. coli RS3 protein
DIDYNTSEAHTTYGVIGVKVWIFKGEILGGMAAVEQPEKPAAQPKKQ

SEQ ID NO: 5 - DNA, 707 bp, Artificial Sequence
  nucleic acid sequence of mutant E. coli RS3 gene made by polymerase assembly multiplexing
atgggacagaaagttcatccgaacggcattcgtctgggcatcgtaaagccttggaatagtacctggttcgctaataccaaagaatttgcagataatctggacagtgacttcaaagttcgtcagtatttaaccaaagaactggctaaagcaagtgttagtcgtattgttattgaacgtccggcaaaaagtattcgtgttaccattcataccgcacgtccgggaatagttattggtaaaaaaggtgaagacgtagaaaaattacgtaaagttgttgcagacatagcaggcgtaccggcacagattaatattgcagaagttcgtaaaccggaattagatgcaaaacttgtcgcagatagtattaccagtcagttagaaagaagagttatgttccgtcgtgcaatgaagagagcagttcagaacgctatgcgtttaggtgcaaaaggtattaaagttgaagttagtggtcgtttaggtggtgcagaaattgcacgtaccgaatggtatcgtgaaggtcgtgttccgttacataccttacgtgcagatattgattataacacaagtgaagcacacactacctatggcgtaattggtgttaaggtatggattttcaagggtgaaattttaggtggtatggcagcagttgaacagccggaaaaaccggcagcacagcacagccgaaaaaacagcagcgtaaaggtcgtaaataa

SEQ ID NO: 6 - DNA, 702 bp, Artificial Sequence
  nucleic acid sequence of E. coli RS3 gene made by polymerase assembly multiplexing
atgggtcagaaagtacatcctaatggtattcgcctgggtattgtaaaaccatggaactctacctggtttgcgaacaccaaagaattcgctgacaacctggacagcgattttaaagtacgtcagtacctgactaaggaactggctaaagcgtccgtatctcgtatcgttatcgagcgtccggctaagagcatccgtgtaaccattcacactgctcgcccgggtatcgttatcggtaaaaaaggtgaagacgtagaaaaactgcgtaaggtcgtagcggacatcgctggcgttcctgcacagatcaacatcgccgaagttcgtaagcctgaactggacgcaaaactggttgctgacagcatcacttctcagctggaacgtcgcgttatgttccgtcgtgctatgaagcgtgctgtacagaacgcaatgcgtctgggcgctaaaggtattaaagttgaagttagcggccgtctgggcggcgcggaaatcgcacgtaccgaatggtaccgcgaaggtcgcgtaccgctgcacactctgcgtgctgacatcgactacaacacctctgaagcgcacaccacttacggtgtaatcggcgttaaagtgtggatcttcaaaggcgagatcctgggtggtatggctgctgttgaacaaccggaaaaaccggctgctcagcctaaaaagcagcagcgtaaaggccgtaaataa

SEQ ID NO: 7 - DNA, 1674 bp, Artificial Sequence
  synthetic oligonucleotide generated by polymerase assembly multiplexing
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggactggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaa

SEQ ID NO: 8 - DNA, 726 bp, Artificial Sequence
  synthetic oligonucleotide generated by polymerase assembly multiplexing
atggcaactgtttccatgcgcgacatgctcaaggctggtgttcacttcggtcaccagacccgttactggaacccgaaaatgaagccgttcatcttcggtgcgcgtaacaaagttcacatcatcaaccttgagaaaactgtaccgatgttcaacgaagctctggctgaactgaacaagattgcttctcgcaaaggtaaaatccttttcgttggtactaaacgcgctgcaagcgaagcggtgaaagacgctgctctgagctgcgaccagttcttcgtgaaccatcgctggctgggcggtatgctgactaactggaaaaccgttcgtcagtccatcaaacgtctgaaagacctggaaactcagtctcaggacggtactttcgacaagctgaccaagaaagaagcgctgatgcgcactcgtgagctggagaaactggaaaacagcctgggcggtatcaaagacatgggcggtctgccggacgctctgtttgtaatcgatgctgaccacgaacacattgctatcaaagaagcaaacaacctgggtattccggtatttgctatcgttgataccaactctgatccggacggtgttgacttcgttatcccgggtaacgacgacgcaatccgtgctgtgaccctgtacctgggcgctgttgctgcaaccgtacgtgaaggccgttctcaggatctggcttcccaggcggaagaaagcttcgtagaagctgagtaa

SEQ ID NO: 9 - DNA, 565 bp, Artificial Sequence
  synthetic oligonucleotide generated by polymerase assembly multiplexing
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacc

SEQ ID NO: 10 - DNA, 697 bp, Artificial Sequence
  synthetic oligonucleotide generated by polymerase assembly multiplexing
tgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggactggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgca

SEQ ID NO: 11 - DNA, 170 bp, Escherichia coli
ggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacc

SEQ ID NO: 12 - DNA, 242 bp, Artificial Sequence
  synthetic oligonucleotide generated by polymerase assembly multiplexing
tgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaa

SEQ ID NO: 13 - DNA, 726 bp, Escherichia coli; identical to SEQ ID NO: 8

SEQ ID NO: 14 - DNA, 54 bp, Artificial Sequence
  synthetic oligonucleotide generated by microchip synt
gtgccgttatcgaatccgaaaacagcgcagagcgcgatcagctgctggaaaacc

SEQ ID NO: 15 - DNA, 54 bp, Artificial Sequence
  synthetic oligonucleotide generated by microchip synt
gggtccgatgactgaatcttttgctcaactctttgaagagtccttaaaagaaat

SEQ ID NO: 16 - DNA, 27 bp, Artificial Sequence
  synthetic oligonucleotide generated by microchip synt
cagagcgcgatcagctgctggaaaacc

SEQ ID NO: 17 - DNA, 13 bp, Artificial Sequence
  synthetic oligonucleotide generated by microchip synt
cagagcgcgatca

SEQ ID NO: 18 - DNA, 14 bp, Artificial Sequence
  synthetic oligonucleotide generated by microchip synt
gctgctggaaaacc

SEQ ID NO: 19 - DNA, 33 bp, Artificial Sequence
  synthetic oligonucleotide generated by microchip synt
gggtccgatgactgaatcttttgctcaactctt

SEQ ID NO: 20 - DNA, 16 bp, Artificial Sequence
  synthetic oligonucleotide generated by microchip synt
gggtccgatgactgaa

SEQ ID NO: 21 - DNA, 17 bp, Artificial Sequence
  synthetic oligonucleotide generated by microchip synt
tcttttgctcaactctt

SEQ ID NO: 22 - DNA, 34 bp, Artificial Sequence
  synthetic oligonucleotide generated by microchip synt
atgactgaatcttttgcatgactgaatcttttgc

SEQ ID NO: 23 - DNA, 28 bp, Artificial Sequence
  synthetic oligonucleotide generated by microchip synt
ggttttccagcagcggttttccagcagc

SEQ ID NO: 24 - DNA, 17 bp, Artificial Sequence
  synthetic oligonucleotide generated by microchip synt
atgactgaatcttttgc

SEQ ID NO: 25 - DNA, 27 bp, Artificial Sequence
  synthetic oligonucleotide generated by microchip synt
cagagcgcgatcagctgctggaaaacc

SEQ ID NO: 26 - DNA, 64 bp, Artificial Sequence
  synthetic oligonucleotide generated by microchip synt
tcagaagcgcaacaacgttgttgtttctcgtcgtgccgttatcgaatccgaaaacagcgcagag

SEQ ID NO: 27 - DNA, 56 bp, Artificial Sequence
  synthetic oligonucleotide generated by microchip synt
tgcgaaaccaatgaaatctctatgactgaatcttttgctcaactctttgaagagtc

SEQ ID NO: 28 - DNA, 40 bp, Artificial Sequence
  synthetic oligonucleotide generated by microchip synt
tccgaaaacagcgcagagcgcgatcagctgctggaaaacc

SEQ ID NO: 29 - DNA, 13 bp, Artificial Sequence
  synthetic oligonucleotide generated by microchip synt
tccgaaaacagcg

SEQ ID NO: 30 - DNA, 14 bp, Artificial Sequence
  synthetic oligonucleotide generated by microchip synt
gctgctggaaaacc

SEQ ID NO: 31 - DNA, 40 bp, Artificial Sequence
  synthetic oligonucleotide generated by microchip synt
tgcgaaaccaatgaaatctctatgactgaatcttttgctc

SEQ ID NO: 32 - DNA, 13 bp, Artificial Sequence
  synthetic oligonucleotide generated by microchip synt
tgcgaaaccaatg

SEQ ID NO: 33 - DNA, 17 bp, Artificial Sequence
  synthetic oligonucleotide generated by microchip synt
gactgaatcttttgctc

SEQ ID NO: 34 - DNA, 15 bp, Artificial Sequence
  synthetic oligonucleotide generated by microchip synt
cggcggtgctcttca

SEQ ID NO: 35 - DNA, 15 bp, Artificial Sequence
  synthetic oligonucleotide generated by microchip synt
gctcggagacctgag

SEQ ID NO: 36 - DNA, 19 bp, Artificial Sequence
  synthetic oligonucleotide generated by microchip synt
atgggacagaaagttcatc

SEQ ID NO: 37 - DNA, 20 bp, Artificial Sequence
  synthetic oligonucleotide generated by microchip synt
ttatttacgacctttacgct

SEQ ID NO: 38 - DNA, 47 bp, Artificial Sequence
  synthetic oligonucleotide generated by microchip synthesis
acctgcgttcggttttccagcagctgatcgcgctctgggagacccta

SEQ ID NO: 39 - DNA, 10 bp, Artificial Sequence
  synthetic oligonucleotide generated by microchip synthesis
acctgcgttc

SEQ ID NO: 40 - DNA, 10 bp, Artificial Sequence
  synthetic oligonucleotide generated by microchip synthesis
ggagacccta

SEQ ID NO: 41 - DNA, 29 bp, Artificial Sequence
  synthetic oligonucleotide generated by microchip synthesis
gctgctggaaaaccgctcggagacctgag

SEQ ID NO: 42 - DNA, 46 bp, Artificial Sequence
  synthetic oligonucleotide generated by microchip synthesis
aaaaaaaaaaaaaaaaagctgctggaaaaccgctcggagacctgag

SEQ ID NO: 43 - DNA, 60 bp, Artificial Sequence
  synthetic oligonucleotide generated by microchip synthesis
cggcggtgctcttcaaaaaaaaaaaaaaaaagctgctggaaaaccgctcggagacctgag

SEQ ID NO: 44 - DNA, 28 bp, Artificial Sequence
  synthetic oligonucleotide generated by microchip synthesis
cagagcgcgatcagctcggagacctgag

SEQ ID NO: 45 - DNA, 46 bp, Artificial Sequence
  synthetic oligonucleotide generated by microchip synthesis
aaaaaaaaaaaaaaaaaacagagcgcgatcagctcggagacctgag

SEQ ID NO: 46 - DNA, 60 bp, Artificial Sequence
  synthetic oligonucleotide generated by microchip synthesis
cggcggtgctcttcaaaaaaaaaaaaaaaaaacagagcgcgatcagctcggagacctgag

SEQ ID NO: 47 - DNA, 27 bp, Artificial Sequence
  synthetic oligonucleotide generated by microchip synthesis
gcgcaacaacgttgttgtttctcgtcg

SEQ ID NO: 48 - DNA, 31 bp, Artificial Sequence
  synthetic oligonucleotide generated by microchip synthesis
tgaagagtccttaaaagaaatcgaaacccgc

SEQ ID NO: 49 - DNA, 27 bp, Artificial Sequence
  synthetic oligonucleotide generated by microchip synthesis
cgttcgtccggtgcgtgacactctgca

SEQ ID NO: 50 - DNA, 27 bp, Artificial Sequence
  synthetic oligonucleotide generated by microchip synthesis
caagctggatcagaagcgcaacaacgt

SEQ ID NO: 51 - DNA, 27 bp, Artificial Sequence
  synthetic oligonucleotide generated by microchip synthesis
cggcaaagttaagggcggcttcactgt

SEQ ID NO: 52 - DNA, 30 bp, Artificial Sequence
  synthetic oligonucleotide generated by microchip synthesis
ccaggttctctggtagacgttcgtccggtg

SEQ ID NO: 53 - DNA, 31 bp, Artificial Sequence
  synthetic oligonucleotide generated by microchip synthesis
gagaaagctaaacgtcacgaagcctggatca

SEQ ID NO: 54 - DNA, 30 bp, Artificial Sequence
  synthetic oligonucleotide generated by microchip synthesis
ttaccggtgttatcaacggcaaagttaagg

SEQ ID NO: 55 - DNA, 30 bp, Artificial Sequence
  synthetic oligonucleotide generated by microchip synthesis
ccaggtaggtgacgaagttgacgttgctct

SEQ ID NO: 56 - DNA, 30 bp, Artificial Sequence
  synthetic oligonucleotide generated by microchip synthesis
tctgctgtcccgtgagaaagctaaacgtca

SEQ ID NO: 57 - DNA, 28 bp, Artificial Sequence
  synthetic oligonucleotide generated by microchip synthesis
ctggtctgaaatctgagtccgccatccc

SEQ ID NO: 58 - DNA, 27 bp, Artificial Sequence
  synthetic oligonucleotide generated by microchip synthesis
gcgagctggaaatccaggtaggtgacg

SEQ ID NO: 59 - DNA, 27 bp, Artificial Sequence
  synthetic oligonucleotide generated by microchip synthesis
atcgaaacccgcccgggttctatcgtt

SEQ ID NO: 60 - DNA, 34 bp, Artificial Sequence
  synthetic oligonucleotide generated by microchip synthesis
cgtagtactggttgacgctggtctgaaatctgag

SEQ ID NO: 61 - DNA, 36 bp, Artificial Sequence
  synthetic oligonucleotide generated by microchip synthesis
ctttgggaatatgactgaatcttttgctcaactctt

SEQ ID NO: 62 - DNA, 19 bp, Artificial Sequence
  synthetic oligonucleotide generated by microchip synthesis
ctttgggaatatgactgaa

SEQ ID NO: 63 - DNA, 708 bp, Artificial Sequence
  synthetic oligonucleotide generated by polymerase assembly multiplexing
tatgggacagaaagttcatccgaacggcattcgtctgggcatcgtaaagccttggaatagtacctggttcgctaataccaaagaatttgcagataatctggacagtgacttcaaagttcgtcagtatttaaccaaagaactggctaaagcaagtgttagtcgtattgttattgaacgtccggcaaaaagtattcgtgttaccattcataccgcacgtccgggaatagttattggtaaaaaaggtgaagacgtagaaaaattacgtaaagttgttgcagacatagcaggcgtaccggcacagattaatattgcagaagttcgtaaaccggaattagatgcaaaacttgtcgcagatagtattaccagtcagttagaaagaagagttatgttccgtcgtgcaatgaagagagcagttcagaacgctatgcgtttaggtgcaaaaggtattaaagttgaagttagtggtcgtttaggtggtgcagaaattgcacgtaccgaatggtatcgtgaaggtcgtgttccgttacataccttacgtgcagatattgattataacacaagtgaagcacacactacctatggcgtaattggtgttaaggtatggattttcaagggtgaaattttaggtggtatggcagcagttgaacagccggaaaaaccggcagcacagcacagccgaaaaaacagcagcgtaaaggtcgtaaataa

SEQ ID NO: 64 - DNA, 44 bp, Artificial Sequence
  synthetic oligonucleotide generated by PCR
tagtagataaacaggaagatgcaaattttagtaatatctatcta

SEQ ID NO: 65 - DNA, 44 bp, Artificial Sequence
  synthetic oligonucleotide generated by PCR
tagatagatattactaaaatttgcatcttcctgtttatctacta

SEQ ID NO: 66 - DNA, 37 bp, Artificial Sequence
  synthetic oligonucleotide generated by PCR
tagtagataaacaggaagatgcaaattttagtaataa

SEQ ID NO: 67 - DNA, 46 bp, Artificial Sequence
  synthetic oligonucleotide generated by PCR
tagatagactttaaatgcttctgccattgcaattattactctacta

SEQ ID NO: 68 - DNA, 68 bp, Artificial Sequence
  synthetic oligonucleotide generated by PCR
tagtagataaacaggaagatgcaaattttagtaataatgcaatggcagaagcatttaaagtctatcta

SEQ ID NO: 69 - DNA, 68 bp, Artificial Sequence
  synthetic oligonucleotide generated by PCR
tagatagactttaaatgcttctgccattgcattattactaaaatttgcatcttcctgtttatctacta

SEQ ID NO: 70 - DNA, 702 bp, Artificial Sequence
  synthetic oligonucleotide generated by polymerase assembly multiplexing
atgggacagaaagttcatccgaacggcattcgtctgggcatcgtaaagccttggaatagtacctggttcgctaataccaaagaatttgcagataatctggacagtgacttcaaagttcgtcagtatttaaccaaagaactggctaaagcaagtgttagtcgtattgttattgaacgtccggcaaaaagtattcgtgttaccattcataccgcacgtccgggaatagttattggtaaaaaaggtgaagacgtagaaaaattacgtaaagttgttgcagacatagcaggcgtaccggcacagattaatattgcagaagttcgtaaaccggaattagatgcaaaacttgtcgcagatagtattaccagtcagttagaaagaagagttatgttccgtcgtgcaatgaagagagcagttcagaacgctatgcgtttaggtgcaaaaggtattaaagttgaagttagtggtcgtttaggtggtgcagaaattgcacgtaccgaatggtatcgtgaaggtcgtgttccgttacataccttacgtgcagatattgattataacacaagtgaagcacacactacctatggcgtaattggtgttaaggtatggattttcaagggtgaaattttaggtggtatggcagcagttgaacagccggaaaaaccggcagcacagccgaaaaaacagcagcgtaaaggtcgtaaataa

SEQ ID NO: 71 - DNA, 306 bp, Artificial Sequence
  synthetic oligonucleotide generated by polymerase assembly multiplexing
atggcaaaacagtcaatgaaagctagagaagttaaacgtgttgcattagcagataaatatttcgctaaacgtgcagaattaaaagcaatcatctcagacgttaatgcatcagacgaagatcgttggaacgcagttttaaaattacagaccttaccgcgtgactcaagtccgagtcgtcagcgtaacagatgtcgtcagaccggcagaccgcatggcttcttacgtaaattcggcttaagtagaatcaaagttcgtgaagcagcaatgcgtggtgaaattccgggtttaaaaaaagcaagttggtaa

SEQ ID NO: 72 - DNA, 70 bp, Artificial Sequence
  synthetic oligonucleotide generated by microchip synt
taaacaggaagatgcaaattttagtaataatgcaatggcagaagcatttaaagcagcaaaaggtgaataa

SEQ ID NO: 73 - DNA, 70 bp, Artificial Sequence
  synthetic oligonucleotide generated by microchip synthesis
agatgaagcagatgaaaaagatgcaattgcaaccgttaataaacaggaagatgcaaattttagtaataat

SEQ ID NO: 74 - DNA, 70 bp, Artificial Sequence
  synthetic oligonucleotide generated by microchip synthesis
ggtgtagaccgtaaaaatcgtgcaattagtttaagtgttcgtgcaaaagatgaagcagatgaaaaagatg

SEQ ID NO: 75 - DNA, 70 bp, Artificial Sequence
  synthetic oligonucleotide generated by microchip synthesis
gcaacccttgtcttaagtgtaggcgatgaagttgaagcaaaatttaccggtgtagaccgtaaaaatcgtg

SEQ ID NO: 76 - DNA, 70 bp, Artificial Sequence
  synthetic oligonucleotide generated by microchip synthesis
aaggctacttacgtgcaagtgaagcaagtcgtgatcgtgttgaagatgcaacccttgtcttaagtgtagg

SEQ ID NO: 77 - DNA, 70 bp, Artificial Sequence
  synthetic oligonucleotide generated by microchip synthesis
cgcagtagacgcaaaaggtgcaactgtagaactggctgacggcgttgaaggctacttacgtgcaagtgaa

SEQ ID NO: 78 - DNA, 70 bp, Artificial Sequence
  synthetic oligonucleotide generated by microchip synthesis
caattgggtagctttaaataaaaaaggtgcaattgttaccggtaaagttaccgcagtagacgcaaaaggt

SEQ ID NO: 79 - DNA, 70 bp, Artificial Sequence
  synthetic oligonucleotide generated by microchip synthesis
tattagtctgggcgtaaagcaactggcagaagacccgtttaacaattgggtagctttaaataaaaaaggt

SEQ ID NO: 80 - DNA, 70 bp, Artificial Sequence
  synthetic oligonucleotide generated by microchip synthesis
acgaaattgcagcagttgttttacaggtagacgcagaacgtgaacgtattagtctgggcgtaaagcaact

SEQ ID NO: 81 - DNA, 70 bp, Artificial Sequence
  synthetic oligonucleotide generated by microchip synthesis
agttggaatgttgcaggtgaagaagctgtacgtgaatataaaaaaggagacgaaattgcagcagttgttt

SEQ ID NO: 82 - DNA, 70 bp, Artificial Sequence
  synthetic oligonucleotide generated by microchip synthesis
ttatcggccttgacggtggcatcgatggtcttgtccatttaagtgacatcagttggaatgttgcaggtga

SEQ ID NO: 83 - DNA, 70 bp, Artificial Sequence
  synthetic oligonucleotide generated by microchip synthesis
taaaggcgaccgtgtagagggtaagattaaaagcattactgactttggcatctttatcggccttgacggt

SEQ ID NO: 84 - DNA, 70 bp, Artificial Sequence
  synthetic oligonucleotide generated by microchip synthesis
ttaaaacagtgcaaggctaacccgtggcagcagttcgctgaaacccataataaaggcgaccgtgtagagg

SEQ ID NO: 85 - DNA, 70 bp, Artificial Sequence
  synthetic oligonucleotide generated by microchip synthesis
gtaatggttttagatatcgacgaagaacgtcgtcgtattagtttaggtttaaaacagtgcaaggctaacc

SEQ ID NO: 86 - DNA, 70 bp, Artificial Sequence
  synthetic oligonucleotide generated by microchip synthesis
atccatccgagtaaagtagtaaacgtaggtgacgtagtggaggtaatggttttagatatcgacgaagaac

SEQ ID NO: 87 - DNA, 70 bp, Artificial Sequence
  synthetic oligonucleotide generated by microchip synthesis
ggccttgttcacgttagtgaaatggactggaccaataaaaacatccatccgagtaaagtagtaaacgtag

SEQ ID NO: 88 - DNA, 70 bp, Artificial Sequence
  synthetic oligonucleotide generated by microchip synthesis
caatttaaccgattatggttgcttcgtagagatcgaggaaggtgtagagggccttgttcacgttagtgaa

SEQ ID NO: 89 - DNA, 70 bp, Artificial Sequence
  synthetic oligonucleotide generated by microchip synthesis
ttgcaaaacgttatccggaaggtaccaaattaaccggcagagttaccaatttaaccgattatggttgctt

SEQ ID NO: 90 - DNA, 70 bp, Artificial Sequence
  synthetic oligonucleotide generated by microchip synthesis
cccgtgttagtttaggattgaaacagttaggtgaagatccgtgggttgcaattgcaaaacgttatccgga

SEQ ID NO: 91 - DNA, 70 bp, Artificial Sequence
  synthetic oligonucleotide generated by microchip synthesis
ggcgacgagataaccgtaaaggttttaaaatttgatcgtgaacgtacccgtgttagtttaggattgaaac

SEQ ID NO: 92 - DNA, 70 bp, Artificial Sequence
  synthetic oligonucleotide generated by microchip synthesis
ccgacatggcatggaaacgtgttaaacatccgagtgaaatcgtaaatgttggcgacgagataaccgtaaa

SEQ ID NO: 93 - DNA, 70 bp, Artificial Sequence
  synthetic oligonucleotide generated by microchip synthesis
ccgattatggtgcatttgtcgacttaggcggcgttgatggtttattacacatcaccgacatggcatggaa

SEQ ID NO: 94 - DNA, 70 bp, Artificial Sequence
  synthetic oligonucleotide generated by microchip synthesis
agaaaatctgcaagaaggtatggaagtaaagggtattgtaaagaatttaaccgattatggtgcatttgtc

SEQ ID NO: 95 - DNA, 70 bp, Artificial Sequence
  synthetic oligonucleotide generated by microchip synthesis
gtgcagtaatcgaaagtgaaaactcagcagaacgtgatcagttattagaaaatctgcaagaaggtatgga

SEQ ID NO: 96 - DNA, 70 bp, Artificial Sequence
  synthetic oligonucleotide generated by microchip synthesis
taatcaaattagatcagaaacgtaacaacgtagtagttagtcgtcgtgcagtaatcgaaagtgaaaactc

SEQ ID NO: 97 - DNA, 70 bp, Artificial Sequence
  synthetic oligonucleotide generated by microchip synthesis
tgataccttacatttagaaggtaaagaattagaatttaaagtaatcaaattagatcagaaacgtaacaac

SEQ ID NO: 98 - DNA, 70 bp, Artificial Sequence
  synthetic oligonucleotide generated by microchip synthesis
ttaccaggcagtttagttgatgttcgtccggttcgtgataccttacatttagaaggtaaagaattagaat

SEQ ID NO: 99 - DNA, 70 bp, Artificial Sequence
  synthetic oligonucleotide generated by microchip synthesis
gtaaaaggcggctttactgttgagttaaatggtattcgtgcatttttaccaggcagtttagttgatgttc

SEQ ID NO: 100 - DNA, 70 bp, Artificial Sequence
  synthetic oligonucleotide generated by microchip synthesis
aaagcatatgaagatgcagaaactgtaaccggtgtaatcaacggcaaggtaaaaggcggctttactgttg

SEQ ID NO: 101 - DNA, 70 bp, Artificial Sequence
  synthetic oligonucleotide generated by microchip synthesis
aagtcgtgaaaaagcaaaacgtcatgaagcatggattaccttagaaaaagcatatgaagatgcagaaact

SEQ ID NO: 102 - DNA, 70 bp, Artificial Sequence
  synthetic oligonucleotide generated by microchip synthesis
agatgtagctttagatgcagtagaggatggcttcggtgaaaccttattaagtcgtgaaaaagcaaaacgt

SEQ ID NO: 103 - DNA, 70 bp, Artificial Sequence
  synthetic oligonucleotide generated by microchip synthesis
aaatgcacagggtgaattagaaattcaggtaggcgatgaggtagatgtagctttagatgcagtagaggat

SEQ ID NO: 104 - DNA, 70 bp, Artificial Sequence
  synthetic oligonucleotide generated by microchip synthesis
atgcaggtttaaaaagtgaaagtgcaattccggcagaacagtttaaaaatgcacagggtgaattagaaat

SEQ ID NO: 105 - DNA, 70 bp, Artificial Sequence
  synthetic oligonucleotide generated by microchip synthesis
gtgcgtggcgtagttgttgctatagacaaagatgttgttttagttgatgcaggtttaaaaagtgaaagtg

SEQ ID NO: 106 - DNA, 70 bp, Artificial Sequence
  synthetic oligonucleotide generated by microchip synthesis
acagttattcgaggaaagtttaaaagaaattgaaacccgtccgggctcaatcgtgcgtggcgtagttgtt

SEQ ID NO: 107 - DNA, 47 bp, Artificial Sequence
  synthetic oligonucleotide generated by microchip synthesis
atgaccgaatcattcgcacagttattcgaggaaagtttaaaagaaat

SEQ ID NO: 108 - DNA, 70 bp, Artificial Sequence
  synthetic oligonucleotide generated by microchip synthesis
ggcagcagttgaacagccggaaaaaccggcagcacagccgaaaaaacagcagcgtaaaggtcgtaaataa

SEQ ID NO: 109 - DNA, 70 bp, Artificial Sequence
  synthetic oligonucleotide generated by microchip synthesis
ggcgtaattggtgttaaggtatggattttcaagggtgaaattttaggtggtatggcagcagttgaacagc

SEQ ID NO: 110 - DNA, 70 bp, Artificial Sequence
  synthetic oligonucleotide generated by microchip synthesis
tacgtgcagatattgattataacacaagtgaagcacacactacctatggcgtaattggtgttaaggtatg

SEQ ID NO: 111 - DNA, 70 bp, Artificial Sequence
  synthetic oligonucleotide generated by microchip synthesis
taccgaatggtatcgtgaaggtcgtgttccgttacataccttacgtgcagatattgattataacacaagt

SEQ ID NO: 112 - DNA, 70 bp, Artificial Sequence
  synthetic oligonucleotide generated by microchip synthesis
gtattaaagttgaagttagtggtcgtttaggtggtgcagaaattgcacgtaccgaatggtatcgtgaagg

SEQ ID NO: 113 - DNA, 70 bp, Artificial Sequence
  synthetic oligonucleotide generated by microchip synthesis
aagagagcagttcagaacgctatgcgtttaggtgcaaaaggtattaaagttgaagttagtggtcgtttag

SEQ ID NO: 114 - DNA, 70 bp, Artificial Sequence
  synthetic oligonucleotide generated by microchip synthesis
gtattaccagtcagttagaaagaagagttatgttccgtcgtgcaatgaagagagcagttcagaacgctat

SEQ ID NO: 115 - DNA, 70 bp, Artificial Sequence
  synthetic oligonucleotide generated by microchip synthesis
gtaaaccggaattagatgcaaaacttgtcgcagatagtattaccagtcagttagaaagaagagttatgtt

SEQ ID NO: 116 - DNA, 70 bp, Artificial Sequence
  synthetic oligonucleotide generated by microchip synthesis
cagacatagcaggcgtaccggcacagattaatattgcagaagttcgtaaaccggaattagatgcaaaact

SEQ ID NO: 117 - DNA, 70 bp, Artificial Sequence
  synthetic oligonucleotide generated by microchip synthesis
tagttattggtaaaaaaggtgaagacgtagaaaaattacgtaaagttgttgcagacatagcaggcgtacc

SEQ ID NO: 118 - DNA, 70 bp, Artificial Sequence
  synthetic oligonucleotide generated by microchip synthesis
aaaaagtattcgtgttaccattcataccgcacgtccgggaatagttattggtaaaaaaggtgaagacgta

SEQ ID NO: 119 - DNA, 70 bp, Artificial Sequence
  synthetic oligonucleotide generated by microchip synthesis
ggctaaagcaagtgttagtcgtattgttattgaacgtccggcaaaaagtattcgtgttaccattcatacc

SEQ ID NO: 120 - DNA, 70 bp, Artificial Sequence
  synthetic oligonucleotide generated by microchip synthesis
tctggacagtgacttcaaagttcgtcagtatttaaccaaagaactggctaaagcaagtgttagtcgtatt

SEQ ID NO: 121 - DNA, 70 bp, Artificial Sequence
  synthetic oligonucleotide generated by microchip synthesis
ccttggaatagtacctggttcgctaataccaaagaatttgcagataatctggacagtgacttcaaagttc

SEQ ID NO: 122 - DNA, 70 bp, Artificial Sequence
  synthetic oligonucleotide generated by microchip synthesis
atgggacagaaagttcatccgaacggcattcgtctgggcatcgtaaagccttggaatagtacctggttcg

SEQ ID NO: 123 - DNA, 20 bp, Artificial Sequence
  synthetic oligonucleotide generated by microchip synthesis
atgggacagaaagttcatcc

SEQ ID NO: 124 - DNA, 70 bp, Artificial Sequence
  synthetic oligonucleotide generated by microchip synthesis
aagtagaatcaaagttcgtgaagcagcaatgcgtggtgaaattccgggtttaaaaaaagcaagttggtaa

SEQ ID NO: 125 - DNA, 70 bp, Artificial Sequence
  synthetic oligonucleotide generated by microchip synthesis
tcgtcagaccggcagaccgcatggcttcttacgtaaattcggcttaagtagaatcaaagttcgtgaagca

SEQ ID NO: 126 - DNA, 70 bp, Artificial Sequence
  synthetic oligonucleotide generated by microchip synthesis
aaaattacagaccttaccgcgtgactcaagtccgagtcgtcagcgtaacagatgtcgtcagaccggcaga

SEQ ID NO: 127 - DNA, 70 bp, Artificial Sequence
  synthetic oligonucleotide generated by microchip synthesis
catctcagacgttaatgcatcagacgaagatcgttggaacgcagttttaaaattacagaccttaccgcgt

SEQ ID NO: 128 - DNA, 70 bp, Artificial Sequence
  synthetic oligonucleotide generated by microchip synthesis
gcattagcagataaatatttcgctaaacgtgcagaattaaaagcaatcatctcagacgttaatgcatcag

SEQ ID NO: 129 - DNA, 70 bp, Artificial Sequence
  synthetic oligonucleotide generated by microchip synthesis
atggcaaaacagtcaatgaaagctagagaagttaaacgtgttgcattagcagataaatatttcgctaaac

SEQ ID NO: 130 - DNA, 22 bp, Artificial Sequence
  synthetic oligonucleotide generated by microchip synthesis
atggcaaaacagtcaatgaaag

SEQ ID NO: 131 - DNA, 70 bp, Artificial Sequence
  synthetic oligonucleotide generated by microchip synthesis
cactccagggtctcgttatttacgacctttacgctgctgttttttcggctgtgctcgtcgcaggtgtcac

SEQ ID NO: 132 - DNA, 70 bp, Artificial Sequence
  synthetic oligonucleotide generated by microchip synthesis
cactccagggtctcgctgttttttcggctgtgctgccggtttttccggctgttcacgtcgcaggtgtcac

SEQ ID NO: 133 - DNA, 70 bp, Artificial Sequence
  synthetic oligonucleotide generated by microchip synthesis
cactccagggtctcggtttttccggctgttcaactgctgccataccacctaaaatcgtcgcaggtgtcac

SEQ ID NO: 134 - DNA, 70 bp, Artificial Sequence
  synthetic oligonucleotide generated by microchip synthesis
cactccagggtctcgctgccataccacctaaaatttcacccttgaaaatccataccgtcgcaggtgtcac

SEQ ID NO: 135 - DNA, 70 bp, Artificial Sequence
  synthetic oligonucleotide generated by microchip synthesis
cactccagggtctcgtcacccttgaaaatccataccttaacaccaattacgccatcgtcgcaggtgtcac

SEQ ID NO: 136 - DNA, 70 bp, Artificial Sequence
  synthetic oligonucleotide generated by microchip synthesis
cactccagggtctcgataccttaacaccaattacgccataggtagtgtgtgcttccgtcgcaggtgtcac

SEQ ID NO: 137 - DNA, 70 bp, Artificial Sequence
  synthetic oligonucleotide generated by microchip synthesis
cactccagggtctcggccataggtagtgtgtgcttcacttgtgttataatcaatacgtcgcaggtgtcac

SEQ ID NO: 138 - DNA, 70 bp, Artificial Sequence
  synthetic oligonucleotide generated by microchip synthesis
cactccagggtctcgtgcttcacttgtgttataatcaatatctgcacgtaaggtacgtcgcaggtgtcac

SEQ ID NO: 139 - DNA, 70 bp, Artificial Sequence
  synthetic oligonucleotide generated by microchip synthesis
cactccagggtctcgtataatcaatatctgcacgtaaggtatgtaacggaacacgcgtcgcaggtgtcac

SEQ ID NO: 140 - DNA, 70 bp, Artificial Sequence
  synthetic oligonucleotide generated by microchip synthesis
cactccagggtctcggtaaggtatgtaacggaacacgaccttcacgataccattccgtcgcaggtgtcac

SEQ ID NO: 141 - DNA, 70 bp, Artificial Sequence
  synthetic oligonucleotide generated by microchip synthesis
cactccagggtctcgcgaccttcacgataccattcggtacgtgcaatttctgcaccgtcgcaggtgtcac

SEQ ID NO: 142 - DNA, 70 bp, Artificial Sequence
  synthetic oligonucleotide generated by microchip synthesis
cactccagggtctcgggtacgtgcaatttctgcaccacctaaacgaccactaactcgtcgcaggtgtcac

SEQ ID NO: 143 - DNA, 70 bp, Artificial Sequence
  synthetic oligonucleotide generated by microchip synthesis
cactccagggtctcgcacctaaacgaccactaacttcaactttaataccttttgccgtcgcaggtgtcac

SEQ ID NO: 144 - DNA, 70 bp, Artificial Sequence
  synthetic oligonucleotide generated by microchip synthesis
cactccagggtctcgcactaacttcaactttaataccttttgcacctaaacgcatcgtcgcaggtgtcac

SEQ ID NO: 145 - DNA, 70 bp, Artificial Sequence
  synthetic oligonucleotide generated by microchip synthesis
cactccagggtctcgaataccttttgcacctaaacgcatagcgttctgaactgctcgtcgcaggtgtcac

SEQ ID NO: 146 - DNA, 70 bp, Artificial Sequence
  synthetic oligonucleotide generated by microchip synthesis
cactccagggtctcggcatagcgttctgaactgctctcttcattgcacgacggaacgtcgcaggtgtcac

SEQ ID NO: 147 - DNA, 70 bp, Artificial Sequence
  synthetic oligonucleotide generated by microchip synthesis
cactccagggtctcgctcttcattgcacgacggaacataactcttctttctaactcgtcgcaggtgtcac

SEQ ID NO: 148 - DNA, 70 bp, Artificial Sequence
  synthetic oligonucleotide generated by microchip synthesis
cactccagggtctcgggaacataactcttctttctaactgactggtaatactatccgtcgcaggtgtcac

SEQ ID NO: 149 - DNA, 70 bp, Artificial Sequence
  synthetic oligonucleotide generated by microchip synthesis
cactccagggtctcgtctttctaactgactggtaatactatctgcgacaagttttcgtcgcaggtgtcac

SEQ ID NO: 150 - DNA, 70 bp, Artificial Sequence
  synthetic oligonucleotide generated by microchip synthesis
cactccagggtctcggtaatactatctgcgacaagttttgcatctaattccggttcgtcgcaggtgtcac

SEQ ID NO: 151 - DNA, 70 bp, Artificial Sequence
  synthetic oligonucleotide generated by microchip synthesis
cactccagggtctcgaagttttgcatctaattccggtttacgaacttctgcaatacgtcgcaggtgtcac

SEQ ID NO: 152 - DNA, 70 bp, Artificial Sequence
  synthetic oligonucleotide generated by microchip synthesis
cactccagggtctcgggtttacgaacttctgcaatattaatctgtgccggtacgccgtcgcaggtgtcac

SEQ ID NO: 153 - DNA, 70 bp, Artificial Sequence
  synthetic oligonucleotide generated by microchip synthesis
cactccagggtctcgatattaatctgtgccggtacgcctgctatgtctgcaacaacgtcgcaggtgtcac

SEQ ID NO: 154 - DNA, 70 bp, Artificial Sequence
  synthetic oligonucleotide generated by microchip synthesis
cactccagggtctcgcctgctatgtctgcaacaactttacgtaatttttctacgtcgtcgcaggtgtcac

SEQ ID NO: 155 - DNA, 70 bp, Artificial Sequence
  synthetic oligonucleotide generated by microchip synthesis
cactccagggtctcgaacaactttacgtaatttttctacgtcttcaccttttttacgtcgcaggtgtcac

SEQ ID NO: 156 - DNA, 70 bp, Artificial Sequence
  synthetic oligonucleotide generated by microchip synthesis
cactccagggtctcgtttctacgtcttcaccttttttaccaataactattcccggcgtcgcaggtgtcac

SEQ ID NO: 157 - DNA, 70 bp, Artificial Sequence
  synthetic oligonucleotide generated by microchip synthesis
cactccagggtctcgaccttttttaccaataactattcccggacgtgcggtatgacgtcgcaggtgtcac

SEQ ID NO: 158 - DNA, 70 bp, Artificial Sequence
  synthetic oligonucleotide generated by microchip synthesis
cactccagggtctcgggacgtgcggtatgaatggtaacacgaatactttttgccgcgtcgcaggtgtcac

SEQ ID NO: 159 - DNA, 70 bp, Artificial Sequence
  synthetic oligonucleotide generated by microchip synthesis
cactccagggtctcgatggtaacacgaatactttttgccggacgttcaataacaacgtcgcaggtgtcac

SEQ ID NO: 160 - DNA, 70 bp, Artificial Sequence
  synthetic oligonucleotide generated by microchip synthesis
cactccagggtctcgccggacgttcaataacaatacgactaacacttgctttagccgtcgcaggtgtcac

SEQ ID NO: 161 - DNA, 70 bp, Artificial Sequence
  synthetic oligonucleotide generated by microchip synthesis
cactccagggtctcgaatacgactaacacttgctttagccagttctttggttaaacgtcgcaggtgtcac

SEQ ID NO: 162 - DNA, 70 bp, Artificial Sequence
  synthetic oligonucleotide generated by microchip synthesis
cactccagggtctcgtttagccagttctttggttaaatactgacgaactttgaagcgtcgcaggtgtcac

SEQ ID NO: 163 - DNA, 70 bp, Artificial Sequence
  synthetic oligonucleotide generated by microchip synthesis
cactccagggtctcgggttaaatactgacgaactttgaagtcactgtccagattacgtcgcaggtgtcac

SEQ ID NO: 164 - DNA, 70 bp, Artificial Sequence
  synthetic oligonucleotide generated by microchip synthesis
cactccagggtctcgtttgaagtcactgtccagattatctgcaaattctttggtacgtcgcaggtgtcac

SEQ ID NO: 165 - DNA, 70 bp, Artificial Sequence
  synthetic oligonucleotide generated by microchip synthesis
cactccagggtctcgagattatctgcaaattctttggtattagcgaaccaggtaccgtcgcaggtgtcac

SEQ ID NO: 166 - DNA, 70 bp, Artificial Sequence
  synthetic oligonucleotide generated by microchip synthesis
cactccagggtctcgtggtattagcgaaccaggtactattccaaggctttacgatcgtcgcaggtgtcac

SEQ ID NO: 167 - DNA, 70 bp, Artificial Sequence
  synthetic oligonucleotide generated by microchip synthesis
cactccagggtctcggtactattccaaggctttacgatgcccagacgaatgccgtcgtcgcaggtgtcac

SEQ ID NO: 168 - DNA, 70 bp, Artificial Sequence
  synthetic oligonucleotide generated by microchip synthesis
cactccagggtctcggcccagacgaatgccgttcggatgaactttctgtcccatacgtcgcaggtgtcac

SEQ ID NO: 169 - DNA, 15 bp, Artificial Sequence
  synthetic oligonucleotide generated by microchip synthesis
cactccagggtctcg

SEQ ID NO: 170 - DNA, 15 bp, Artificial Sequence
  synthetic oligonucleotide generated by microchip synthesis
gtgacacctgcgacg

SEQ ID NO: 171 - DNA, 70 bp, Artificial Sequence; identical to SEQ ID NO: 131
  synthetic oligonucleot ide generated by PCR

SEQ ID NO: 172 - DNA, 70 bp, Artificial Sequence
  synthetic oligonucleot ide generated by PCR
gagacacctgcgacgagcacagccgaaaaaacagcagcgtaaaggtcgtaaataacgagaccctggagtg

(19) United States
(12) Patent Application Publication
(10) Pub. No.: US 2006/0127920 A1
(43) Pub. Date: Jun. 15, 2006

(54) Polynucleotide synthesis

(75) Inventors: George M. Church, Brookline, MA (US); Jingdong Tian, Arlington, MA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 11/069,910

(22) Filed: Feb. 28, 2005

Related U.S. Application Data
(60) Provisional application No. 60/636,672, filed on Dec. 16, 2004.
(60) Provisional application No. 60/600,957, filed on Aug. 12, 2004.
(60) Provisional application No. 60/548,637, filed on Feb. 27, 2004.

(51) Int. Cl. C12Q 1/68; C12P 19/34; C12M 1/34

(52) U.S. Cl. 435/6; 435/91.2; 435/287.2

(57) ABSTRACT

Methods of improving the kinetics of bimolecular interactions where reactants are present in low concentrations are provided. Methods of pre-amplifying one or more oligonucleotides using high concentration universal primers are provided. Methods of improving the error rate in oligonucleotide and/or polynucleotide syntheses are also provided. Methods for sequence optimization and oligonucleotides design are further provided.

### RELATED U.S. APPLICATIONS

This application claims priority to U.S. Provisional Patent Application Ser. Nos. 60/548,637 filed on Feb. 27, 2004; 60/600,957 filed on Aug. 12, 2004; and 60/636,672, filed on Dec. 16, 2004, hereby incorporated by reference in their entirety for all purposes.

### STATEMENT OF GOVERNMENT INTERESTS

This invention was made with Government support under Award Number F30602-01-2-0586 awarded by The Defense Advanced Research Projects Agency (DARPA). The Government has certain rights in the invention.

### FIELD OF THE INVENTION

The present invention relates to methods of making synthetic polynucleotides.

### BACKGROUND OF THE INVENTION

The advance of large-scale biochemical analyses such as sequencing, microarrays and proteomics has generated vast amounts of data, which computational biologists have leveraged into a large number of hypotheses. However, the bottleneck in constructing new genetic elements, genetic pathways and engineered cells must be overcome. To optimize complex biological processes using Darwinian selection, the finite diversity available in combinatorial oligonucleotide synthesis (about 25 randomized base pairs (bp) or equivalents) needs to be directed thoughtfully through large stretches (at the megabase level) of DNA sequence. These represent great challenges and potential payoffs for the emerging field of synthetic biology.

Methods are available in the art to create a useful variety of molecules, cellular and cell-free systems given a sufficient supply of custom genes and genomes. However, current methods for generating even simple oligonucleotides are expensive (US $0.11 per nucleotide) and have very high levels of errors (deletions at a rate of 1 in 100 bases and mismatches and insertions at a rate of about 1 in 400 bases). As a result, gene or genome synthesis from oligonucleotides is both expensive and prone to error. Correcting errors by clone sequencing and mutagenesis methods further increases the amount of labor and total cost (to at least US $2 per base pair).

The cost of oligonucleotide synthesis can be reduced by performing massively parallel custom syntheses on microchips (Zhou et al. (2004) Nucleic Acids Res. 32:5409; Fodor et al. (1991) Science 251:767). This can be achieved using a variety of methods, including ink-jet printing with standard reagents (Agilent; see e.g., U.S. Pat. No. 6,323,043), photolabile 5′ protecting groups (Nimbelgen/Affymetrix; see e.g., U.S. Pat. No. 5,405,783; and PCT Publication Nos. WO 03/065038; 03/064699; WO 03/064026; 02/04597), photo-generated acid deprotection (e.g., Atactic and Xeotron technologies, see e.g., X. Gao et al., Nucleic Acids Res. 29: 4744-50 (2001); X. Gao et al., J. Am. Chem. Soc. 120: 12698-12699 (1998); O. Srivannavit et al., Sensors and Actuators A. 116: 150-160 (2004); and U.S. Pat. No. 6,426,184) and electrolytic acid/base arrays (Oxamer/Combimatrix; see e.g., U.S. Patent Publication No. 2003/0054344; U.S. Pat. Nos. 6,093,302; 6,444,111; 6,280,595). However, current microchips have very low surface areas and hence only small amounts of oligonucleotides can be produced. When released into solution, the oligonucleotides are present at pictomolar or lower concentrations per sequence, concentrations that are insufficiently high to drive bimolecular priming reactions efficiently.

The manufacture of accurate DNA constructs is severely impacted by error rates inherent in chemical synthesis techniques. As FIG. 1 illustrates, by way of example, in a DNA embodying an open reading frames comprising 3000 base pairs, synthesized by a method having an error rate of 1 base in 1000, less than 5% of the copies of the synthesized DNA will be correct.

A state of the art oligonucleotide synthesizer exploiting phosphoamidite chemistry makes errors at a rate of approximately one base in 200. DNAs synthesized on chips using photo labile synthesis techniques reportedly have an error rate of about 1/50, and potentially may be improved to about 1/100. High fidelity PCR has an error rate of about 1/10⁵. Even at such high fidelity duplication, for a gene 3000 bp in length, a polymerases operating ex vivo produce copies that contain an error about 3% of the time. Because the current best commercial DNA synthesis protocols represent the pinnacle of several decades of development, it seems unlikely that order of magnitude additional improvements in chemical synthesis of polynucleotides will be forthcoming in the near future.

The widespread use of gene and genome synthesis technology is hampered by limitations such as high cost and high error rate, and lack of automation. Practical, economical methods of synthesizing custom polynucleotides, large genetic systems, and methods of producing synthetic polynucleotides that have lower error rates than synthetic polynucleotides made by methods known in the art are needed.

### SUMMARY

Broadly, the invention enables cost-effective production of useful, high fidelity synthetic DNA constructs by providing a group of improvements to the DNA assembly methods of Mullis (Mullis et al. (1986) Cold Spring Harb. Symp. Quant. Biol. 51 Pt 1:263) and Stemmer (Stemmer et al. (1995) Gene 164:49) which may be used individually or together. The improvements include advances in computational design of the oligonucleotides used for assembly, i.e., in the design of the “construction oligonucleotides” and for purification, i.e., the “selection oligonucleotides,” multiplexing of construction oligonucleotide assembly, i.e., making plural different assemblies in the same pool, construction oligonucleotide amplification techniques, and construction oligonucleotide error reduction techniques.

In one embodiment, the invention provides methods for preparing a polynucleotide construct having a predefined sequence involving amplification of the oligonucleotides at various stages. The method comprises providing a pool of construction oligonucleotides having (i) partially overlapping sequences that define the sequence of the polynucleotide construct, (ii) at least one pair of primer hybridization sites flanking at least a portion of said construction oligonucleotides and common to at least a subset of said construction oligonucleotides, and (iii) cleavage sites between the primer hybridization sites and the construction oligonucleotides. The pool of construction oligonucleotides may then be amplified using at least one primer that binds to the primer hybridization sites. Optionally, the primer hybridization sites may then be removed from the construction oligonucleotides at the cleavage sites (e.g., using a restriction endonuclease, chemical cleavage, etc.). After amplification, the construction oligonucleotides may then be subjected to assembly, e.g., by denaturing the oligonucleotides to separate the complementary strands and then exposing the pool of construction oligonucleotides to hybridization conditions and ligation and/or chain extension conditions.

In another embodiment, the invention provides methods for preparing a purified pool of construction oligonucleotides. The methods comprise contacting a pool of construction oligonucleotides with a pool of selection oligonucleotides under hybridization conditions to form duplexes. The reaction will form both stable duplexes (e.g., duplexes comprising a copy of a construction oligonucleotide and a copy of a selection oligonucleotide that do not contain a mismatch in the complementary region) and unstable duplexes (e.g., duplexes comprising a copy of a construction oligonucleotide and a copy of a selection oligonucleotide that contain one or more mismatches, e.g., base mismatches, insertions, or deletion, in the complementary region). The copies of the construction oligonucleotides that formed unstable duplexes may then be removed from the pool (e.g., using a separation technique such as a column) to form a pool of purified construction oligonucleotides. Optionally, the purification process (e.g., mixture of the construction and selection oligonucleotides) may be repeated at least once before use of the construction oligonucleotides. Additionally, the pool of construction oligonucleotides may be amplified before and/or after the various rounds of purification by selection. After forming the pool of purified construction oligonucleotides, they pool may be subjected to assembly conditions. For example, the pool of construction oligonucleotides may be exposed to hybridization conditions and ligation and/or chain extension conditions.

In another embodiment, the invention provides methods for preparing a plurality of polynucleotide constructs having different predefined sequences in a single pool. The method comprises (i) providing a pool of construction oligonucleotides comprising partially overlapping sequences that define the sequence of each of said plurality of polynucleotide constructs and (ii) incubating said pool of construction oligonucleotides under hybridization conditions and ligation and/or chain extension conditions. Optionally, the oligonucleotides and/or polynucleotide constructs may be subjected to one or more rounds of amplification and/or error reduction as desired. Additionally, the polynucleotide constructs may be subject to further rounds of assembly to produce even longer polynucleotide constructs. At least about 2, 4, 5, 10, 50, 100, 1,000 or more polynucleotide constructs may be assembled in a single pool.

In another embodiment, the invention provides methods for designing construction and/or selection oligonucleotides as well as an assembly strategy for producing one or more polynucleotide constructs. The method may comprise, for example, (i) computationally dividing the sequence of each polynucleotide construct into partially overlapping sequence segments; (ii) synthesizing construction oligonucleotides comprising sequences corresponding to the sets of partially overlapping sequence segments; and (iii) incubating said construction oligonucleotides under hybridization conditions and ligation and/or chain extension conditions. Optionally, the method may further comprise (i) computationally adding to the termini of at least a portion of said construction oligonucleotides one or more pairs of primer hybridization sites common to at least a subset of said construction oligonucleotides and defining cleavage sites between the primer hybridization sites and the construction oligonucleotides; (ii) amplifying said construction oligonucleotides using at least one primer that binds to said primer hybridization sites; and (iii) removing said primer hybridization sites from said construction oligonucleotides at said cleavage sites. Preferably such primer sites may be common to at least a portion of the construction oligonucleotides in the pool. The method may further comprise computationally designing at least one pool of selection oligonucleotides comprising sequences that are complementary to at least portions of said construction oligonucleotides, synthesizing said selection oligonucleotides, and conduction an error filtration process by hybridization the pool of construction oligonucleotides to the pool of selection oligonucleotides.

Embodiments of the present invention are also directed to methods for assembling plural different polynucleotide sequences in a single pool. These methods include the steps of providing a group of synthetic oligonucleotides having complementary terminal regions and primer sites flanking the oligonucleotides comprising the ends of said different polynucleotide sequences, mixing the synthetic oligonucleotides together with dNTPs and a polymerase, and cycling the mixture to induce hybridization of the complementary terminal regions, polymerase mediated incorporation of bases to extend overlapping oligonucleotides and to produce copies of full length different polynucleotide sequences, and amplification of multiple said full length sequences.

In certain aspects, such methods also include the use of plural separate pools, at least some of the different synthetic polynucleotide sequences thereby produced in each pool comprising polynucleotides having complementary terminal regions and primer sites flanking the different polynucleotide sequences comprising the ends of said larger polynucleotides. At least some of the plural pools are mixed together with dNTPs and a polymerase, and the mixture is cycled to induce hybridization of complementary terminal regions of the different polynucleotide sequences. polymerase mediated incorporation of bases is used to extend overlapping polynucleotide sequences and to produce copies of full length larger polynucleotides, and amplification of multiple said full length larger polynucleotides.

In certain aspects, synthetic oligonucleotides are synthesized in parallel by serial automated parallel assembly of plural base sequences and purified (e.g., purification by hybridization) to reduce the concentration of oligonucleotide copies embodying sequence errors. In other aspects, the synthetic oligonucleotides are synthesized on a surface. In still other aspects, plural pairs of the complementary terminal regions are designed to have similar melting temperatures. In yet other aspects, the pool is a well or a microchannel. In other aspects, the mixing step is conducted by flowing the components of said mixture together in a microfluidic system wherein said polymerase is a thermally stable polymerase.

Embodiments of the present invention are directed to articles of manufacture including a multiplicity of different, retrievable polynucleotides. The articles include a polynucleotide reservoir which contains a mixture of different polynucleotides comprising differing pairs of primer sequences which permit amplification of a subgroup of said different polynucleotides from the reservoir, and plural primer reservoirs each of which contains a pair of oligonucleotide primers complementary to a pair of primer sequences of a polynucleotide in the construct reservoir. The primer sequence pairs of polynucleotides in a polynucleotide reservoir can be different from each other. The polynucleotides can comprise synthetic DNA, genes, multiple mutants of a wild-type sequence, vectors and the like. at least a portion of said polynucleotides are at least one kilobase long. In certain aspects, at least a portion of the polynucleotides are at least two kilobases long, at least five kilobases long, at least ten kilobases long, or longer.

In certain aspects, the polynucleotides can be circularized. The polynucleotides can optionally be flanked by adapter sequences to facilitate manipulation of the polynucleotide sequence, such as insertion into a vector, immobilization, or identification of a function of the sequence. The polynucleotides can include one or more sequences selected from the group consisting of mammalian sequences, yeast sequences, prokaryotic sequences, plant sequences, D. melanogaster sequences, C. elegans sequences, and Xenopus sequences.

In other aspects, the mixture of different, retrievable polynucleotide constructs are independently retrievable. For example, the article of manufacture may include plural polynucleotide reservoirs containing plural different polynucleotides, the polynucleotides in different reservoirs comprising an identical said pair of primer sequences, wherein one or more of said plural primer reservoirs contain a pair of said complementary oligonucleotide primers. A polynucleotide reservoir can contain D different independently retrievable polynucleotides each of which comprise N nested primer pairs, the number of primer reservoirs being at least N/2×D^{1/N}, or can contain D different polynucleotides and D primer reservoirs containing pairs of primers. A polynucleotide reservoir can contain different polynucleotides comprising plural nested pairs of primer sequences, each of said plural nested pairs permitting amplification of a selected group of polynucleotides in said reservoir or of individual ones of said different polynucleotides therein. The article of manufacture can contain 10² different polynucleotides, 10³ different polynucleotides, 10⁴ different polynucleotides, 10⁵ different polynucleotides, 10⁶ different polynucleotides or more.

Embodiments of the present invention are further directed to articles of manufacture comprising a package containing a multiplicity of different, retrievable polynucleotides. The articles include a polynucleotide reservoir which contains a mixture of different polynucleotides at least some of which comprise plural nested pairs of primer sequences, each of the plural nested pairs permitting amplification of a selected group of polynucleotides in the reservoir or of individual ones of said different polynucleotides therein. The articles also include plural primer reservoirs each of which contains a pair of oligonucleotide primers complementary to a pair of primer sequences of a polynucleotide in said construct reservoir. The combination of nested pairs on each polynucleotide in the reservoir can be different from the combination of nested pairs of all other polynucleotides in the reservoir. The article can include plural construct reservoirs each of which contains plural different polynucleotides, polynucleotides in different reservoirs comprising an identical pair of primer sequences so that a given primer pair anneals with different polynucleotides in different reservoirs.

Embodiments of the present invention are also directed to apparatuses for supplying a solution rich in a selected one of or a selected group of polynucleotide constructs. The apparatuses include a polynucleotide reservoir which contains a mixture of identified polynucleotides comprising at least one pair of primer sequences which permit amplification of selected ones of said different polynucleotides from said reservoir and being different from other pairs of primer sequence of other polynucleotides in said reservoir and plural primer reservoirs each of which contains a pair of oligonucleotide primers complementary to a pair of primer sequences of a different polynucleotide in the construct reservoirs. The apparatuses also include data storage listing the identified polynucleotides and the position of the one or more reservoirs containing the primer pair or pairs complementary to the respective identified polynucleotides and an interface permitting a user to specify a polynucleotide or group of polynucleotides. The apparatuses further include automated means responsive to specifications input at the interface and instructions accessed from the data storage for extracting aliquots of polynucleotides from the construct reservoir and primers from selected primer reservoirs to prepare reagents needed to amplify selectively said specified polynucleotide or group of polynucleotides.

In certain aspects, the apparatuses include plural polynucleotide reservoirs which contain different identified polynucleotides. In other aspects, polynucleotides in different reservoirs comprise the same pair of primer sequences. In still other aspects, polynucleotides in different reservoirs comprise plural nested pairs of primer sequences comprising at least 10 polynucleotide reservoirs. In yet other aspects, polynucleotides in different reservoirs comprise unique nested pairs of primer sequences.

The apparatuses can include an amplification chamber adapted to amplify a selected identified polynucleotide retrieved from the construct reservoir as specified by a selected primer pair. In other aspects, the apparatuses also include a second amplification chamber adapted to amplify one or a subgroup of identified polynucleotides retrieved from the amplification chamber as specified by a selected primer pair.

Embodiments of the present invention are also directed to methods of obtaining a polynucleotide of choice. The methods include providing plural construct reservoirs containing mixtures of identified synthesized polynucleotides comprising plural nested pairs of primer sequences which permit amplification of selected ones of said polynucleotides from a said reservoir, the combination of primer pairs of a polynucleotide in a said reservoir being different from other pairs of primer sequence of other polynucleotides in said reservoir. Then plural primer reservoirs each of which contains a pair of oligonucleotide primers complementary to a pair of primer sequences of a polynucleotide in the construct reservoirs are provided. A first amplification procedure is conducted in a first amplification mixture comprising an aliquot of a the mixture of polynucleotides retrieved from a selected construct reservoir and a pair of primers complementary to an outer nested pair of primer sequences retrieved from one or more primer reservoirs. A second amplification procedure is conducted in a second amplification mixture comprising an aliquot of amplicons retrieved from the first amplification mixture and a pair of primers complementary to an inner nested pair of primer sequences retrieved from one or more primer reservoirs.

Embodiments of the present invention are also directed to multiplicities of synthesized polynucleotides in admixture forming a library. The library includes a multiplicity of polynucleotide species, at least some of the species having an outer pair of primer sequences of a length sufficient to permit amplification of selected groups of species retrieved from the library. The library also includes an inner pair of primer sequences having a length sufficient to permit amplification of one or selected groups of species retrieved from a mixture of amplicons produced by amplification using said outer pair. In certain aspects, a concentration of an individual species in the library is insufficient to permit selective amplification thereof directly from the library but sufficient to permit selective amplification thereof after amplification using the outer primer sequence pair. In another aspect, the synthesized polynucleotides comprise three nested pairs of primer sequences. In another aspect, the synthesized polynucleotides each comprise nested pairs of primer sequences having a different nucleic acid sequence than all other nested pairs of primer sequences in the library.

The methods described herein are also useful for generating libraries of variant sequences for functional screening and selection.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A-1C depict preparation of free oligonucleotides from a customary microarray. (A) depicts a diagram of synthesis and cleavage of a PCR-amplifiable oligonucleotide from a microchip surface. The portion of the oligonucleotide used for gene construction is depicted in black; PCR-primer adaptors are shown in grey. (B) depicts synthesis and cleavage of oligonucleotides from a Xeotron/Atactic 4K photo-programmable microfluidic microchip. Left: fluorescent scanning micrograph of an oligonucleotides array before cleavage. Insert: details of microfluidic chambers and connecting channels. Right: array after cleavage. (C) depicts hybridization of released fluorescein (FAM)-labelled oligonucleotides to a quality assessment (QA)-chip. Left: prior to hybridization; middle: after hybridization; right: after stripping of hybridized nucleotides.

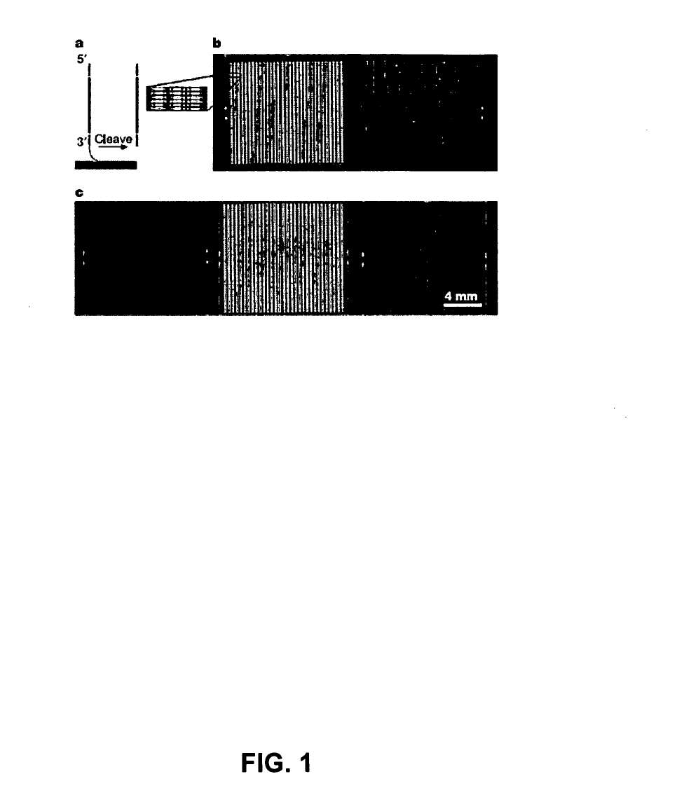

FIGS. 2A-2B depict the amino acid sequences of new RS3 vs. original E. coli K12. 2A is set forth as SEQ ID NO:1; 2B upper is set forth as SEQ ID NO:2; 2B middle is set forth as SEQ ID NO:3; 2B lower is set forth as SEQ ID NO:4.

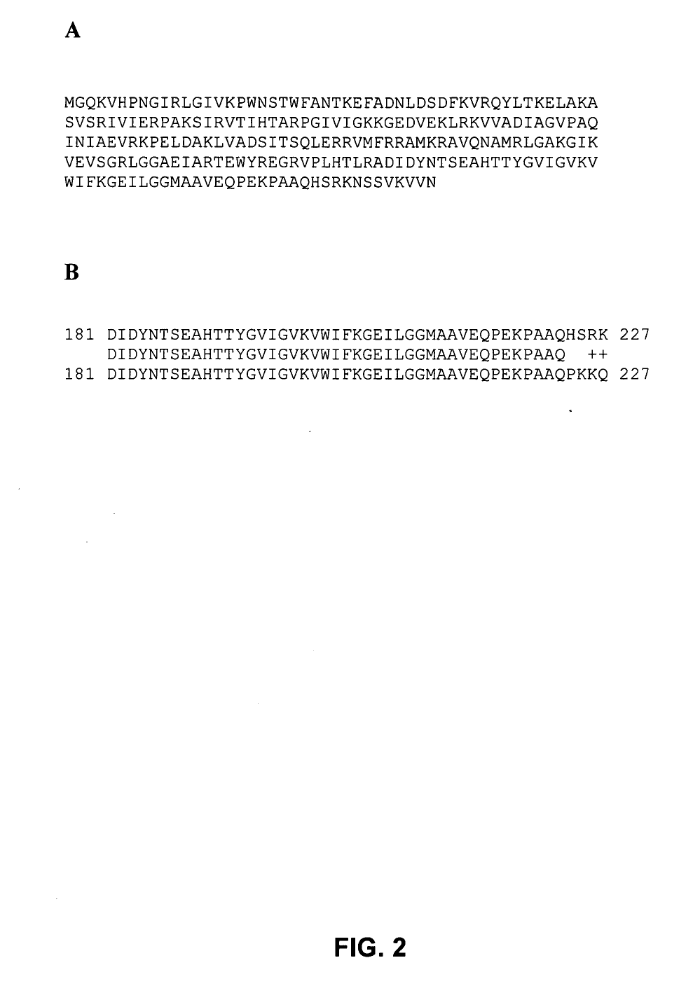

FIG. 3 depicts the nucleic acid sequences of new RS3 vs. original E. coli K12. Score=212 bits (107), Expect=6e-52, Identities=557/707 (78%), Gaps=5/707 (0%). Upper sequence is set forth as SEQ ID NO:5; lower sequence is set forth as SEQ ID NO:6.

FIG. 4 depicts an agarose gel showing 21 synthesized rs gene T7-expression constructs.

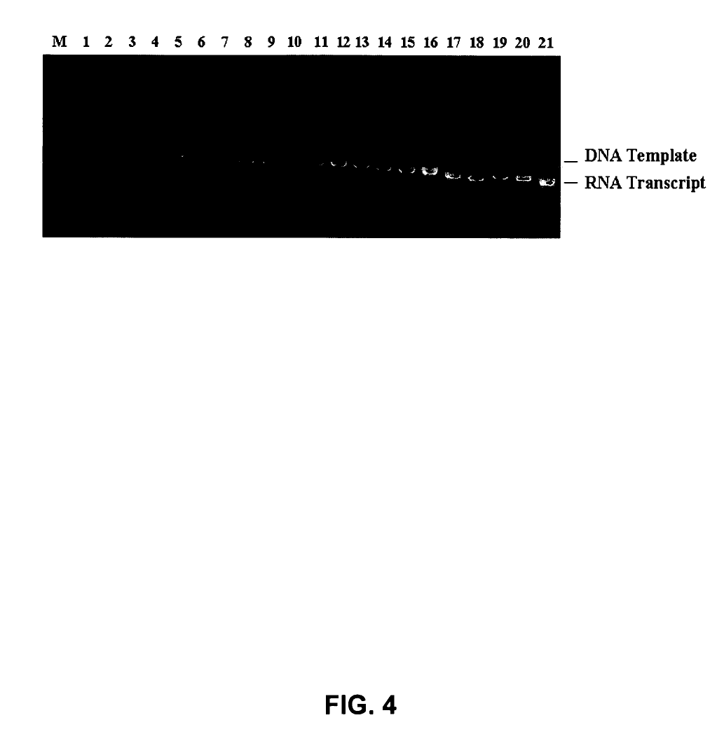

FIG. 5 depicts a diagram of the hybridization strategy for hybridization selection of microchip-synthesized oligonucleotides. 90-mer oligonucleotides (upper strands black, lower strands grey) are cut with type IIS restriction enzymes to release hybrids of 50-mers and complementary 44-mers, some of which have incorrect sequences (indicated by a bulge in the upper strand of the second 90-mer oligonucleotide). Only the correct upper 50-mer strand hybridizes well with left (L) then right (R) selection oligonucleotides (immobilized on beads in grey).

FIG. 6 depicts a flow chart for the design, synthesis and analysis of multiple genes in pools. Estimates of current process timing (not always the minimum possible times) are listed.

FIG. 7 depicts a flow chart showing operation of a program for designing oligonucleotides according to certain embodiments of the invention.

FIG. 8 depicts an exemplary input sequences file for the program of FIG. 7. Rs1 is set forth as SEQ ID NO:7; rs2 is set forth as SEQ ID NO:8.

FIGS. 9A-9B depicts an exemplary parameters input file for the program of FIG. 7.

FIGS. 10A-10B depict exemplary codon usage tables for the program of FIG. 7.

FIG. 11 depicts a flow chart showing optimization of an input sequence according to certain embodiments of the invention.

FIG. 12 depicts one of the sequences from FIG. 8 after restriction enzyme cleavage. Rs1-f1 is set forth as SEQ ID NO:9; rs1-f2 is set forth as SEQ ID NO:10; rs1-f3 is set forth as SEQ ID NO:11; rs1-f4 is set forth as SEQ ID NO:12; rs2-f1 is set forth as SEQ ID NO:13.

FIGS. 13A-13B depict flow charts showing selection of oligonucleotide fragments based on melting point (Tₘ) according to certain embodiments of the invention.

FIG. 14 depicts a diagram illustrating the selection algorithm of FIGS. 13A-13B. Sequence is set forth as SEQ ID NO:9.

FIG. 15 depicts a diagram illustrating the selection algorithm of FIGS. 13A-13B. Sequence is set forth as SEQ ID NO:14.

FIG. 16 depicts a diagram illustrating the selection algorithm of FIGS. 13A-13B. Sequence is set forth as SEQ ID NO:14.

FIG. 17 depicts a diagram illustrating the selection algorithm of FIGS. 13A-13B. Sequence is set forth as SEQ ID NO:15.

FIG. 18 depicts an example of data output for the algorithm of FIGS. 13A-13B. Rs1-f1-1 is set forth as SEQ ID NO:16; rs1-f1-1L is set forth as SEQ ID NO:17; rs1-f1-1R is set forth as SEQ ID NO:18; rs1-f1-38 is set forth as SEQ ID NO:19; rs1-f1-38L is set forth as SEQ ID NO:20; rs1-f1-38R is set forth as SEQ ID NO:21; rs1-f1-L is set forth as SEQ ID NO:22; rs1-f1-R is set forth as SEQ ID NO:23; left primer is set forth as SEQ ID NO:24; right primer is set forth as SEQ ID NO:25.

FIG. 19 depicts a flow chart showing selection of oligonucleotide fragments based on length according to certain embodiments of the invention.

FIG. 20 depicts a diagram illustrating the selection algorithm of FIG. 19. Sequence is set forth as SEQ ID NO:14.

FIG. 21 depicts a diagram illustrating the selection algorithm of FIG. 19. Sequence is set forth as SEQ ID NO:26.

FIG. 22 depicts a diagram illustrating the selection algorithm of FIG. 19. Sequence is set forth as SEQ ID NO:27.

FIG. 23 is an example of data output for the algorithm of FIG. 19. Rs1-f1-1 is set forth as SEQ ID NO:28; rs1-f1-1L is set forth as SEQ ID NO:29 rs1-f1-1R is set forth as SEQ ID NO:30; rs1-f1-23 is set forth as SEQ ID NO:31; rs1-f1-23L is set forth as SEQ ID NO:32; rs1-f1-23R is set forth as SEQ ID NO:33; rs1-f1-L is set forth as SEQ ID NO:22; rs1-f1-R is set forth as SEQ ID NO:23; left primer is set forth as SEQ ID NO:24; right primer is set forth as SEQ ID NO:28.

FIG. 24 diagrammatically depicts how construction oligonucleotides are designed according to certain embodiments of the invention. Rs1-f1-1 is set forth as SEQ ID NO:16; rs1-f1-1L is set forth as SEQ ID NO:17; rs1-f1-1R is set forth as SEQ ID NO:18; rs1-f1-1c is set forth as SEQ ID NO:38; sense5endAddOn is set forth as SEQ ID NO:39; sense3endAddOn is set forth as SEQ ID NO:40.

FIG. 25 diagrammatically depicts how selection oligonucleotides are designed according to certain embodiments of the invention. Sequence (1) is set forth as SEQ ID NO:38; sequence (2) is set forth as SEQ ID NO:37; sequence (3) is set forth as SEQ ID NO:41; sequence (4) is set forth as SEQ ID NO:42; sequence (5) is set forth as SEQ ID NO:43; sequence (6) is set forth as SEQ ID NO:36; sequence (7) is set forth as SEQ ID NO:44; sequence (8) is set forth as SEQ ID NO:45; sequence (9) is set forth as SEQ ID NO:46.

FIG. 26 depicts an exemplary program output when a different poolSize parameter is specified. Rs1-f1-1 is set forth as SEQ ID NO:35; rs1-f1-1L is set forth as SEQ ID NO:36; rs1-a1-1R is set forth as SEQ ID NO:37; pool-1 left primer is set forth as SEQ ID NO:47; pool-1 right primer is set forth as SEQ ID NO:23; pool-2 left primer is set forth as SEQ ID NO:49; pool-2 right primer is set forth as SEQ ID NO:50; pool-3 left primer is set forth as SEQ ID NO:51; pool-3 right primer is set forth as SEQ ID NO:52; pool-4 left primer is set forth as SEQ ID NO:53; pool-4 right primer is set forth as SEQ ID NO:54; pool-5 left primer is set forth as SEQ ID NO:55; pool-5 right primer is set forth as SEQ ID NO:56; pool-6 left primer is set forth as SEQ ID NO:57; pool-6 right primer is set forth as SEQ ID NO:58; pool-7 left primer is set forth as SEQ ID NO:59; pool-7 right primer is set forth as SEQ ID NO:60; pool-8 left primer is set forth as SEQ ID NO:24; pool-8 right primer is set forth as SEQ ID NO:48.

FIG. 27 depicts an exemplary program output when a different chipExtraSeqLen parameter is specified. Rs1-f1-1 is set forth as SEQ ID NO:35; rs1-f1-1L is set forth as SEQ ID NO:36; rs1-f1-1R is set forth as SEQ ID NO:37; rs1-f1-38 is set forth as SEQ ID NO:61; rs1-f1-38L is set forth as SEQ ID NO:62; rs1-f1-38R is set forth as SEQ ID NO:21; rs1-f1-L is set forth as SEQ ID NO:22; rs1-f1-R is set forth as SEQ ID NO:23; left primer is set forth as SEQ ID NO:24; right primer is set forth as SEQ ID NO:25.

FIG. 28 depicts the effects of error rates on polynucleotide fidelity.

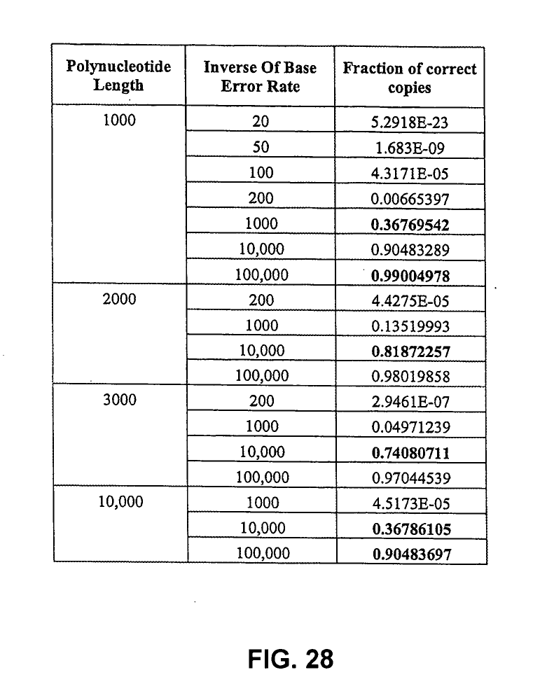

FIG. 29 depicts a schematic overview of one embodiment of a method for multiplex assembly of multiple polynucleotide constructs, from design of oligonucleotides to the production of a plurality of polynucleotide constructs having a predetermined sequence.

FIG. 30 depicts a schematic overview of three exemplary methods for assembly of construction oligonucleotides into subassemblies and/or polynucleotide constructs, including (A) ligation, (B) chain extension and (C) chain extension and ligation. The dotted lines represent strands that have been extended by polymerase.

FIG. 31 depicts a schematic overview of one embodiment of a method for polynucleotide assembly that involves multiple rounds of assembly.

FIG. 32 depicts a schematic overview of one embodiment of a method for polynucleotide assembly that utilizes universal primers to amplify an oligonucleotide pool.

FIG. 33 depicts a schematic overview demonstrating one embodiment of a method for polynucleotide assembly that utilizes one set of universal primers to amplify a pool of construction oligonucleotides and one set of universal primers to amplify a subassembly (e.g., abc).

FIG. 34 depicts one method for removal of error sequences using mismatch binding proteins.

FIG. 35 depicts neutralization of error sequences with mismatch recognition proteins.

FIG. 36 depicts one method for strand-specific error correction.

FIG. 37 depicts a schematic overview demonstrating one method for increasing the efficiency of error reduction processes by subjecting an oligonucleotide pool to a round of denaturation/renaturation prior to error reduction. Xs represent sequence errors (e.g., deviations from a desired sequence in the form of an insertion, deletion, or incorrect base).

FIG. 38 depicts a comparison of sequence errors generated by various methods. χ² tests were performed for hybridization selection versus PAGE selection (P=2×10⁻⁵), and hybridization selection versus no selection (P=2×10⁻²¹). Only the constructs in the row labeled ‘PAGE Selection’ involved gel purification.

### DETAILED DESCRIPTION

The present invention provides an economical method of synthesizing custom polynucleotides, and a method of producing synthetic oligonucleotides and/or polynucleotides that have lower mismatch error rates than oligonucleotides and/or polynucleotides made by methods known in the art.

One major advance of the methods described herein over methods known in the art is the ability to use the small number of molecules available from surface oligonucleotide array syntheses. The methods provided herein exploit two further strategies to improve the kinetics of bimolecular interactions where reactants are present in low concentrations. In one embodiment, the present invention provides a method of pre-amplifying one or more oligonucleotides using high concentration “universal” primers. In another embodiment, the present invention provides a method of exploiting the initially high concentrations of the oligonucleotides at the time of synthesis.

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “amplification” means that the number of copies of a nucleic acid fragment is increased.

The term “base-pairing” refers to the specific hydrogen bonding between purines and pyrimidines in double-stranded nucleic acids including, for example, adenine (A) and thymine (T), guanine (G) and cytosine (C), (A) and uracil (U), and guanine (G) and cytosine (C), and the complements thereof. Base-pairing leads to the formation of a nucleic acid double helix from two complementary single strands.

The term “cleavage” as used herein refers to the breakage of a bond between two nucleotides, such as a phosphodiester bond.

The terms “comprise” and “comprising” are used in the inclusive, open sense, meaning that additional elements may be included.

The term “construction oligonucleotide” refers to a single stranded oligonucleotide that may be used for assembling nucleic acid molecules that are longer than the construction oligonucleotide itself. In exemplary embodiments, a construction oligonucleotide may be used for assembling a nucleic acid molecule that is at least about 3-fold, 4-fold, 5-fold, 10-fold, 20-fold, 50-fold, 100-fold, or more, longer than the construction oligonucleotide. Typically a set of different construction oligonucleotides having predetermined sequences will be used for assembly into a larger nucleic acid molecule having a desired sequence. In exemplary embodiments, construction oligonucleotides may be from about 25 to about 200, about 50 to about 150, about 50 to about 100, or about 50 to about 75 nucleotides in length. Assembly of construction oligonucleotides may be carried out by a variety of methods including, for example, PAM, PCR assembly, ligation chain reaction, ligation/fusion PCR, dual asymmetrical PCR, overlap extension PCR, and combinations thereof. Construction oligonucleotides may be single stranded oligonucleotides or double stranded oligonucleotides. In an exemplary embodiment, construction oligonucleotides are synthetic oligonucleotides that have been synthesized in parallel on a substrate. Sequence design for construction oligonucleotides may be carried out with the aid of a computer program such as, for example, DNAWorks (Hoover and Lubkowski, Nucleic Acids Res. 30: e43 (2002), Gene2oligo (Rouillard et al., Nucleic Acids Res. 32: W176-180 (2004) and world wide web at berry.engin.umich.edu/gene2oligo), or the implementation systems and methods discussed further below.

The term “dam” refers to an adenine methyltransferases that plays a role in coordinating DNA replication initiation, DNA mismatch repair and the regulation of expression of some genes. The term is meant to encompass prokaryotic dam proteins as well as homologs, orthologs, paralogs, variants, or fragments thereof. Exemplary dam proteins include, for example, polypeptides encoded by nucleic acids having the following GenBank accession Nos. AF091142 (Neisseria meningitidus strain BF13), AF006263 (Treponema pallidum), U76993 (Salmonella typhimurium) and M22342 (Bacteriphage T2).

The terms “denature” or “melt” refer to a process by which strands of a duplex nucleic acid molecule are separated into single stranded molecules. Methods of denaturation include, for example, thermal denaturation and alkaline denaturation.

The term “detectable marker” refers to a polynucleotide sequence that facilitates the identification of a cell harboring the polynucleotide sequence. In certain embodiments, the detectable marker encodes for a chemiluminescent or fluorescent protein, such as, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). In other embodiments, the detectable marker may be an antigenic or affinity tag such as, for example, a polyHis tag, myc, HA, GST, protein A, protein G, calmodulin-binding peptide, thioredoxin, maltose-binding protein, poly arginine, poly His-Asp, FLAG, and the like.

The term “duplex” refers to a nucleic acid molecule that is at least partially double stranded. A “stable duplex” refers to a duplex that is relatively more likely to remain hybridized to a complementary sequence under a given set of hybridization conditions. In an exemplary embodiment, a stable duplex refers to a duplex that does not contain a base pair mismatch, insertion, or deletion. An “unstable duplex” refers to a duplex that is relatively less likely to remain hybridized to a complementary sequence under a given set of hybridization conditions. In an exemplary embodiment, an unstable duplex refers to a duplex that contains at least one base pair mismatch, insertion, or deletion.

The term “error reduction” refers to process that may be used to reduce the number of sequence errors in a nucleic acid molecule, or a pool of nucleic acid molecules, thereby increasing the number of error free copies in a composition of nucleic acid molecules. Error reduction includes error filtration, error neutralization, and error correction processes. “Error filtration” is a process by which nucleic acid molecules that contain a sequence error are removed from a pool of nucleic acid molecules. Methods for conducting error filtration include, for example, hybridization to a selection oligonucleotide, or binding to a mismatch binding agent, followed by separation. “Error neutralization” is a process by which a nucleic acid containing a sequence error is restricted from amplifying and/or assembling but is not removed from the pool of nucleic acids. Methods for error neutralization include, for example, binding to a mismatch binding agent and optionally covalent linkage of the mismatch binding agent to the DNA duplex. “Error correction” is a process by which a sequence error in a nucleic acid molecule is corrected (e.g., an incorrect nucleotide at a particular location is changed to the nucleic acid that should be present based on the predetermined sequence). Methods for error correction include, for example, homologous recombination or sequence correction using DNA repair proteins.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “hybridize” or “hybridization” refers to specific binding between two complementary nucleic acid strands. In various embodiments, hybridization refers to an association between two perfectly matched complementary regions of nucleic acid strands as well as binding between two nucleic acid strands that contain one or more mismatches (including mismatches, insertion, or deletions) in the complementary regions. Hybridization may occur, for example, between two complementary nucleic acid strands that contain 1, 2, 3, 4, 5 or more mismatches. In various embodiments, hybridization may occur, for example, between partially overlapping and complementary construction oligonucleotides, between partially overlapping and complementary construction and selection oligonucleotides, between a primer and a primer binding site, etc. The stability of hybridization between two nucleic acid strands may be controlled by varying the hybridization conditions and/or wash conditions, including for example, temperature and/or salt concentration. For example, the stringency of the hybridization conditions may be increased so as to achieve more selective hybridization, e.g., as the stringency of the hybridization conditions are increased the stability of binding between two nucleic acid strands, particularly strands containing mismatches, will be decreased.

The term “including” is used to mean “including but not limited to”. “Including” and “including but not limited to” are used interchangeably.

The term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double helix, i.e. where the ligation process ligates a “nick” at a ligatable nick site and creates a complementary duplex (Blackburn, M. and Gait, M. (1996) in Nucleic Acids in Chemistry and Biology, Oxford University Press, Oxford, pp. 132-33, 481-2). The site between the adjacent oligonucleotides is referred to as the “ligatable nick site”, “nick site”, or “nick”, whereby the phosphodiester bond is non-existent, or cleaved.

The term “ligate” refers to the reaction of covalently joining adjacent oligonucleotides through formation of an internucleotide linkage.

The term “selectable marker” refers to a polynucleotide sequence encoding a gene product that alters the ability of a cell harboring the polynucleotide sequence to grow or survive in a given growth environment relative to a similar cell lacking the selectable marker. Such a marker may be a positive or negative selectable marker. For example, a positive selectable marker (e.g., an antibiotic resistance or auxotrophic growth gene) encodes a product that confers growth or survival abilities in selective medium (e.g., containing an antibiotic or lacking an essential nutrient). A negative selectable marker, in contrast, prevents polynucleotide-harboring cells from growing in negative selection medium, when compared to cells not harboring the polynucleotide. A selectable marker may confer both positive and negative selectability, depending upon the medium used to grow the cell. The use of selectable markers in prokaryotic and eukaryotic cells is well known by those of skill in the art. Suitable positive selection markers include, e.g., neomycin, kanamycin, hyg, hisD, gpt, bleomycin, tetracycline, hprt SacB, beta-lactamase, ura3, ampicillin, carbenicillin, chloramphenicol, streptomycin, gentamycin, phleomycin, and nalidixic acid. Suitable negative selection markers include, e.g., hsv-tk, hprt, gpt, and cytosine deaminase.

The term “selection oligonucleotide” refers to a single stranded oligonucleotide that is complementary to at least a portion of a construction oligonucleotide (or the complement of the construction oligonucleotide). Selection oligonucleotides may be used for removing copies of a construction oligonucleotide that contain sequencing errors (e.g., a deviation from the desired sequence) from a pool of construction oligonucleotides. In an exemplary embodiment, a selection oligonucleotide may be end immobilized on a substrate. In one embodiment, selection oligonucleotides are synthetic oligonucleotides that have been synthesized in parallel on a substrate. Selection oligonucleotides can be complementary to at least about 20%, 25%, 30%, 50%, 60%, 70%, 80%, 90%, or 100% of the length of the construction oligonucleotide (or the complement of the construction oligonucleotide). In an exemplary embodiment, a pool of selection oligonucleotides is designed such that the melting temperature (Tₘ) of a plurality of construction/selection oligonucleotide pairs is substantially similar. In one embodiment, a pool of selection oligonucleotides is designed such that the melting temperature of substantially all of the construction/selection oligonucleotides pairs is substantially similar. For example, the melting temperature of at least about 50%, 60%, 70%, 75%, 80%, 90%, 95%, 97%, 98%, 99%, or greater, of the construction/selection oligonucleotide pairs is within about 10° C., 7° C., 5° C., 4° C., 3° C., 2° C., 1° C., or less, of each other. Sequence design for selection oligonucleotides may be carried out with the aid of a computer program such as, for example, DNAWorks (Hoover and Lubkowski, Nucleic Acids Res. 30: e43 (2002), Gene2Oligo (Rouillard et al., Nucleic Acids Res. 32: W176-180 (2004) and world wide web at berry.engin.umich.edu/gene2oligo), or the implementation systems and methods discussed further below.

The terms “stringent conditions” or “stringent hybridization conditions” refer to conditions which promote specific hybridization between two complementary polynucleotide strands so as to form a duplex. Stringent conditions may be selected to be about 5° C. lower than the thermal melting point (Tₘ) for a given polynucleotide duplex at a defined ionic strength and pH. The length of the complementary polynucleotide strands and their GC content will determine the Tₘ of the duplex, and thus the hybridization conditions necessary for obtaining a desired specificity of hybridization. The Tₘ is the temperature (under defined ionic strength and pH) at which 50% of a polynucleotide sequence hybridizes to a perfectly matched complementary strand. In certain cases it may be desirable to increase the stringency of the hybridization conditions to be about equal to the Tₘ for a particular duplex.

A variety of techniques for estimating the Tₘ are available. Typically, G-C base pairs in a duplex are estimated to contribute about 3° C. to the Tₘ, while A-T base pairs are estimated to contribute about 2° C., up to a theoretical maximum of about 80-100° C. However, more sophisticated models of Tₘ are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)-562)/#bp)-5; where #GC, #AT, and #bp are the number of guanine-cytosine base pairs, the number of adenine-thymine base pairs, and the number of total base pairs, respectively, involved in the formation of the duplex. Other methods for calculating Tₘ are described in SantaLucia and Hicks, Ann. Rev. Biomol. Struct. 33: 415-40 (2004) using the formula Tₘ=ΔH^{o}×1000/(ΔS^{o}+R×ln(C_{T}/x))−273.15, where C_{T} is the total molar strand concentration, R is the gas constant 1.9872 cal/K-mol, and x equals 4 for nonself-complementary duplexes and equals 1 for self-complementary duplexes.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature. In an exemplary embodiment, Betaine, e.g., about 5 M Betaine, may be added to the hybridization reaction to minimize or eliminate the base pair composition dependence of DNA thermal melting transitions (see e.g., Rees et al., Biochemistry 32: 137-144 (1993)). In another embodiment, low molecular weight amides or low molecule weight sulfones (such as, for example, DMSO, tetramethylene sulfoxide, methyl sec-butyl sulfoxide, etc.) may be added to a hybridization reaction to reduce the melting temperature of sequences rich in GC content (see e.g., Chakarbarti and Schutt, BioTechniques 32: 866-874 (2002)).

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y.; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York; Tibanyenda, N. et al., Eur. J. Biochem. 139:19 (1984) and Ebel, S. et al., Biochem. 31:12083 (1992); Rees et al., Biochemistry 32: 137-144 (1993); Chakarbarti and Schutt, BioTechniques 32: 866-874 (2002); and SantaLucia and Hicks, Annu. Rev. Biomol. Struct. 33: 415-40 (2004).

As applied to proteins, the term “substantial identity” means that two sequences, when optimally aligned, such as by the programs GAP or BESTFIT using default gap weights, typically share at least about 70 percent sequence identity, alternatively at least about 80, 85, 90, 95 percent sequence identity or more. For amino acid sequences, amino acid residues that are not identical may differ by conservative amino acid substitutions, which are described above.

The term “subassembly” refers to a nucleic acid molecule that has been assembled from a set of construction oligonucleotides. Preferably, a subassembly is at least about 3-fold, 4-fold, 5-fold, 10-fold, 20-fold, 50-fold, 100-fold, or more, longer than the construction oligonucleotide, e.g., about 300-600 bases long.

The term “synthetic,” as used herein with reference to a nucleic acid molecule, refers to production by in vitro chemical and/or enzymatic synthesis.

“Transcriptional regulatory sequence” is a generic term used herein to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

As used herein, the term “transfection” means the introduction of a nucleic acid, e.g., an expression vector, into a recipient cell, and is intended to include commonly used terms such as “infect” with respect to a virus or viral vector. The term “transduction” is generally used herein when the transfection with a nucleic acid is by viral delivery of the nucleic acid. The term “transformation” refers to any method for introducing foreign molecules, such as DNA, into a cell. Lipofection, DEAE-dextran-mediated transfection, microinjection, protoplast fusion, calcium phosphate precipitation, retroviral delivery, electroporation, natural transformation, and biolistic transformation are just a few of the methods known to those skilled in the art which may be used.

The term “universal primers” refers to a set of primers (e.g., a forward and reverse primer) that may be used for chain extension/amplification of a plurality of polynucleotides, e.g., the primers hybridize to sites that are common to a plurality of polynucleotides. For example, universal primers may be used for amplification of all, or essentially all, polynucleotides in a single pool, such as, for example, a pool of construction oligonucleotides, a pool of selection oligonucleotides, a pool of subassemblies, and/or a pool of polynucleotide constructs, etc. In one embodiment, a single primer may be used to amplify both the forward and reverse strands of a plurality of polynucleotides in a single pool. In certain embodiments, the universal primers may be temporary primers that may be removed after amplification via enzymatic or chemical cleavage. In other embodiments, the universal primers may comprise a modification that becomes incorporated into the polynucleotide molecules upon chain extension. Exemplary modifications include, for example, a 3′ or 5′ end cap, a label (e.g., fluorescein), or a tag (e.g., a tag that facilitates immobilization or isolation of the polynucleotide, such as, biotin, etc.).

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

Embodiments of the present invention are directed to methods of generating and amplifying synthetic oligonucleotide sequences such as construction oligonucleotides and selection oligonucleotides. As used herein, the term “oligonucleotide” is intended to include, but is not limited to, a single-stranded DNA or RNA molecule, typically prepared by synthetic means. Nucleotides of the present invention will typically be the naturally-occurring nucleotides such as nucleotides derived from adenosine, guanosine, uridine, cytidine and thymidine. When oligonucleotides are referred to as “double-stranded,” it is understood by those of skill in the art that a pair of oligonucleotides exists in a hydrogen-bonded, helical array typically associated with, for example, DNA. In addition to the 100% complementary form of double-stranded oligonucleotides, the term “double-stranded” as used herein is also meant to include those form which include such structural features as bulges and loops (see Stryer, Biochemistry, Third Ed. (1988), incorporated herein by reference in its entirety for all purposes). As used herein, the term “polynucleotide” is intended to include, but is not limited to, two or more oligonucleotides joined together (e.g., by hybridization, ligation, polymerization and the like).

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “polynucleotide construct” refers to a long nucleic acid molecule having a predetermined sequence. Polynucleotide constructs may be assembled from a set of construction oligonucleotides and/or a set of subassemblies.

The term “restriction endonuclease recognition site” refers to a nucleic acid sequence capable of binding one ore more restriction endonucleases. The term “restriction endonuclease cleavage site” refers to a nucleic acid sequence that is cleaved by one or more restriction endonucleases. For a given enzyme, the restriction endonuclease recognition and cleavage sites may the same or different. Restriction enzymes include, but are not limited to, type I enzymes, type II enzymes, type IIS enzymes, type III enzymes and type IV enzymes.

In certain aspects of the invention, nucleotide analogs or derivatives will be used, such as nucleosides or nucleotides having protecting groups on either the base portion or sugar portion of the molecule, or having attached or incorporated labels, or isosteric replacements which result in monomers that behave in either a synthetic or physiological environment in a manner similar to the parent monomer. The nucleotides can have a protecting group which is linked to, and masks, a reactive group on the nucleotide. A variety of protecting groups are useful in the invention and can be selected depending on the synthesis techniques employed and are discussed further below. After the nucleotide is attached to the support or growing nucleic acid, the protecting group can be removed.

As used herein the term “construction oligonucleotide” is intended to include, but is not limited to, an oligonucleotide sequence that is identical or complementary to a target nucleic acid sequence (e.g. a gene) or a portion thereof.

As used herein the term “selection oligonucleotide” is intended to include, but is not limited to, an oligonucleotide sequence that is complementary to at least a portion of construction oligonucleotide, and can hybridize to that portion in a sequence specific manner.

Oligonucleotides or fragments thereof may be isolated from natural sources or purchased from commercial sources. Oligonucleotide sequences may be prepared by any suitable method, e.g., the phosphoramidite method described by Beaucage and Carruthers ((1981) Tetrahedron Lett. 22: 1859) or the triester method according to Matteucci et al. (1981) J. Am. Chem. Soc. 103:3185), both incorporated herein by reference in their entirety for all purposes, or by other chemical methods using either a commercial automated oligonucleotide synthesizer or high-throughput, high-density array methods described herein and known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides and chips containing oligonucleotides may also be obtained commercially from a variety of vendors.

In various embodiments, the methods described herein utilize construction and/or selection oligonucleotides. The sequences of the construction and/or selection oligonucleotides will be determined based on the sequence of the final polynucleotide construct that is desired to be synthesized. Essentially the sequence of the polynucleotide construct may be divided up into a plurality of overlapping shorter sequences that can then be synthesized in parallel and assembled into the final desired polynucleotide construct using the methods described herein. Design of the construction and/or selection oligonucleotides may be facilitated by the aid of a computer program such as, for example, DNAWorks (Hoover and Lubkowski (2002) Nuc. Acids Res. 30:e43, Gene2Oligo (Rouillard et al., Nucleic Acids Res. 32:W176-180 (2004) and world wide web at berry.engin.umich.edu/gene2oligo), or CAD-PAM software described further below. In certain embodiments, it may be desirable to design a plurality of construction oligonucleotide/selection oligonucleotide pairs to have substantially similar melting temperatures in order to facilitate manipulation of the plurality of oligonucleotides in a single pool. This process may be facilitated by the computer programs described above. Normalizing melting temperatures between a variety of oligonucleotide sequences may be accomplished by varying the length of the oligonucleotides and/or by codon remapping the sequence (e.g., varying the A/T vs. G/C content in one or more oligonucleotides without altering the sequence of a polynucleotide that may ultimately be encoded thereby) (see e.g., WO 99/58721).

In certain embodiments, the construction oligonucleotides are designed to provide essentially the full complement of sense and antisense strands of the desired polynucleotide construct. For example, the construction oligonucleotides merely need to be hybridized together and subjected to ligation in order to form the full polynucleotide construct. In other embodiments, the complement of construction oligonucleotides may be designed to cover the full sequence, but leave single stranded gaps that may be filed in by chain extension prior to ligation. This embodiment will facilitate production of polynucleotide constructs because it requires synthesis of fewer and/or shorter construction oligonucleotides and/or selection oligonucleotides.

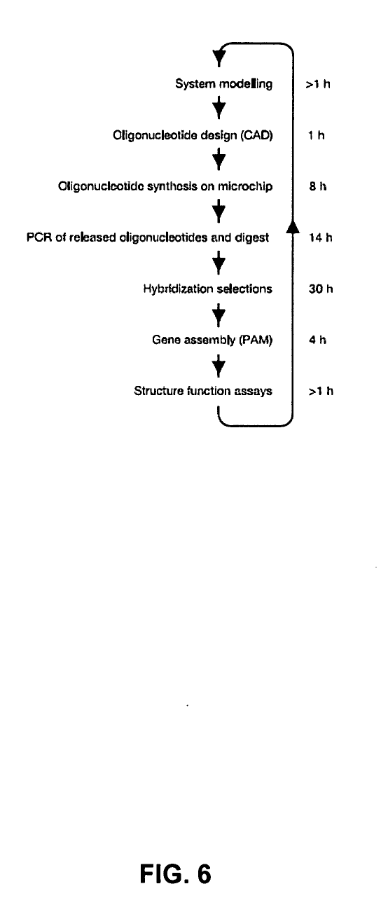

In an exemplary embodiment, construction and/or selection oligonucleotides may comprise one or more sets of binding sites for universal primers that may be used for amplification of a pool of nucleic acids with one set, or a few sets, of primers. The sequence of the universal primer binding sites may be chosen to have an appropriate length and sequence to permit efficient primer hybridization and chain extension. Additionally, the sequence of the universal primer binding sites may be optimized so as to minimize non-specific binding to an undesired region of a nucleic acid in the pool. Design of universal primers and binding sites for the universal primers may be facilitated using a computer program such as, for example, DNAWorks (supra), Gene2Oligo (supra), or the implementation systems and methods discussed further below. In certain embodiments, it may be desirable to design several sets of universal primers/primer binding sites that will permit amplification of nucleic acids at different stages of polynucleotide construction (FIG. 6). For example, one set of universal primers may be used to amplify a set of construction and/or selection oligonucleotides. After assembly of a set of construction oligonucleotides into a subassembly, the subassembly may be amplified using the same or a different set of universal primers. For example, the 3′ and 5′ most terminal construction oligonucleotides that are incorporated into the subassembly may contain two or more nested sets of universal primer binding sites, the outermost set which may be used for initial amplification of the construction oligos and second set that may be used to amplify the subassembly. It is possible to incorporate multiple sets of universal primers for amplification at each stage of an assembly (e.g., construction and/or selection oligonucleotides, subassemblies, and/or polynucleotide constructs).

In exemplary embodiments, the universal primers may be designed as temporary primers, e.g., primers that can be removed from the nucleic acid molecule by chemical or enzymatic cleavage. Methods for chemical, thermal, light based, or enzymatic cleavage of nucleic acids are described in detail below. In an exemplary embodiment, the universal primers may be removed using a Type IIS restriction endonuclease.

Construction and/or selection oligonucleotides may be prepared by any method known in the art for preparation of oligonucleotides having a desired sequence. For example, oligonucleotides may be isolated from natural sources, purchased from commercial sources, or designed from first principals. Preferably, oligonucleotides may be synthesized using a method that permits high-throughput, parallel synthesis so as to reduce cost and production time and increase flexibility. In an exemplary embodiment, construction and/or selection oligonucleotides may be synthesized on a solid support in an array format, e.g., a microarray of single stranded DNA segments synthesized in situ on a common substrate wherein each oligonucleotide is synthesized on a separate feature or location on the substrate. Arrays may be constructed, custom ordered, or purchased from a commercial vendor. Various methods for constructing arrays are well known in the art. For example, methods and techniques applicable to synthesis of construction and/or selection oligonucleotide synthesis on a solid support, e.g., in an array format have been described, for example, in WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752 and Zhou et al., Nucleic Acids Res. 32: 5409-5417 (2004).

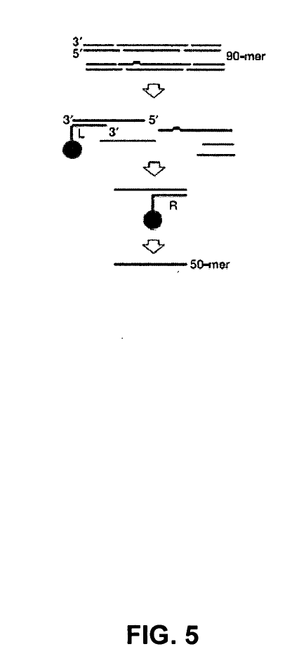

In an exemplary embodiment, construction and/or selection oligonucleotides may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence. The preferred type of instrument is the type shown in FIG. 5 of U.S. Pat. No. 6,375,903, based on the use of reflective optics. It is a desirable that this type of maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no different to the process. The MAS instrument may be used in the form it would normally be used to make microarrays for hybridization experiments, but it may also be adapted to have features specifically adapted for the compositions, methods, and systems described herein. For example, it may be desirable to substitute a coherent light source, i.e. a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization.

Other methods synthesizing construction and/or selection oligonucleotides include, for example, light-directed methods utilizing masks, flow channel methods, spotting methods, pin-based methods, and methods utilizing multiple supports.

Light directed methods utilizing masks (e.g., VLSIPS™ methods) for the synthesis of oligonucleotides is described, for example, in U.S. Pat. Nos. 5,143,854, 5,510,270 and 5,527,681. These methods involve activating predefined regions of a solid support and then contacting the support with a preselected monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in U.S. Pat. No. 5,384,261.

Additional methods applicable to synthesis of construction and/or selection oligonucleotides on a single support are described, for example, in U.S. Pat. No. 5,384,261. For example reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. One of skill in the art will recognize that there are alternative methods of forming channels or otherwise protecting a portion of the surface of the support. For example, a protective coating such as a hydrophilic or hydrophobic coating (depending upon the nature of the solvent) is utilized over portions of the support to be protected, sometimes in combination with materials that facilitate wetting by the reactant solution in other regions. In this manner, the flowing solutions are further prevented from passing outside of their designated flow paths.

Spotting methods for preparation of oligonucleotides on a solid support involve delivering reactants in relatively small quantities by directly depositing them in selected regions. In some steps, the entire support surface can be sprayed or otherwise coated with a solution, if it is more efficient to do so. Precisely measured aliquots of monomer solutions may be deposited dropwise by a dispenser that moves from region to region. Typical dispensers include a micropipette to deliver the monomer solution to the support and a robotic system to control the position of the micropipette with respect to the support, or an ink-jet printer. In other embodiments, the dispenser includes a series of tubes, a manifold, an array of pipettes, or the like so that various reagents can be delivered to the reaction regions simultaneously.

Pin-based methods for synthesis of oligonucleotides on a solid support are described, for example, in U.S. Pat. No. 5,288,514. Pin-based methods utilize a support having a plurality of pins or other extensions. The pins are each inserted simultaneously into individual reagent containers in a tray. An array of 96 pins is commonly utilized with a 96-container tray, such as a 96-well microtitre dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

In yet another embodiment, a plurality of construction and/or selection oligonucleotides may be synthesized on multiple supports. On example is a bead based synthesis method which is described, for example, in U.S. Pat. Nos. 5,770,358, 5,639,603, and 5,541,061. For the synthesis of molecules such as oligonucleotides on beads, a large plurality of beads are suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

Various exemplary protecting groups useful for synthesis of oligonucleotides on a solid support are described in, for example, Atherton et al., 1989, Solid Phase Peptide Synthesis, IRL Press.

In various embodiments, the methods described herein utilize solid supports for immobilization of nucleic acids. For example, oligonucleotides may be synthesized on one or more solid supports. Additionally, selection oligonucleotides may be immobilized on a solid support to facilitate removal of construction oligonucleotides containing sequence errors. Exemplary solid supports include, for example, slides, beads, chips, particles, strands, gels, sheets, tubing, spheres, containers, capillaries, pads, slices, films, or plates. In various embodiments, the solid supports may be biological, nonbiological, organic, inorganic, or combinations thereof. When using supports that are substantially planar, the support may be physically separated into regions, for example, with trenches, grooves, wells, or chemical barriers (e.g., hydrophobic coatings, etc.). Supports that are transparent to light are useful when the assay involves optical detection (see e.g., U.S. Pat. No. 5,545,531). The surface of the solid support will typically contain reactive groups, such as carboxyl, amino, and hydroxyl or may be coated with functionalized silicon compounds (see e.g., U.S. Pat. No. 5,919,523).

In one embodiment, the oligonucleotides synthesized on the solid support may be used as a template for the production of construction oligonucleotides and/or selection oligonucleotides for assembly into longer polynucleotide constructs. For example, the support bound oligonucleotides may be contacted with primers that hybridize to the oligonucleotides under conditions that permit chain extension of the primers. The support bound duplexes may then be denatured and subjected to further rounds of amplification.

In another embodiment, the support bound oligonucleotides may be removed from the solid support prior to assembly into polynucleotide constructs. The oligonucleotides may be removed from the solid support, for example, by exposure to conditions such as acid, base, oxidation, reduction, heat, light, metal ion catalysis, displacement or elimination chemistry, or by enzymatic cleavage.

In one embodiment, oligonucleotides may be attached to a solid support through a cleavable linkage moiety. For example, the solid support may be functionalized to provide cleavable linkers for covalent attachment to the oligonucleotides. The linker moiety may be of six or more atoms in length. Alternatively, the cleavable moiety may be within an oligonucleotide and may be introduced during in situ synthesis. A broad variety of cleavable moieties are available in the art of solid phase and microarray oligonucleotide synthesis (see e.g., Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728). A suitable cleavable moiety may be selected to be compatible with the nature of the protecting group of the nucleoside bases, the choice of solid support, and/or the mode of reagent delivery, among others. In an exemplary embodiment, the oligonucleotides cleaved from the solid support contain a free 3′-OH end. Alternatively, the free 3′-OH end may also be obtained by chemical or enzymatic treatment, following the cleavage of oligonucleotides. The cleavable moiety may be removed under conditions which do not degrade the oligonucleotides. Preferably the linker may be cleaved using two approaches, either (a) simultaneously under the same conditions as the deprotection step or (b) subsequently utilizing a different condition or reagent for linker cleavage after the completion of the deprotection step.

The covalent immobilization site may either be at the 5′ end of the oligonucleotide or at the 3′ end of the oligonucleotide. In some instances, the immobilization site may be within the oligonucleotide (i.e. at a site other than the 5′ or 3′ end of the oligonucleotide). The cleavable site may be located along the oligonucleotide backbone, for example, a modified 3′-5′ internucleotide linkage in place of one of the phosphodiester groups, such as ribose, dialkoxysilane, phosphorothioate, and phosphoramidate internucleotide linkage. The cleavable oligonucleotide analogs may also include a substituent on, or replacement of, one of the bases or sugars, such as 7-deazaguanosine, 5-methylcytosine, inosine, uridine, and the like.

In one embodiment, cleavable sites contained within the modified oligonucleotide may include chemically cleavable groups, such as dialkoxysilane, 3′-(S)-phosphorothioate, 5′-(S)-phosphorothioate, 3′-(N)-phosphoramidate, 5′-(N)phosphoramidate, and ribose. Synthesis and cleavage conditions of chemically cleavable oligonucleotides are described in U.S. Pat. Nos. 5,700,642 and 5,830,655. For example, depending upon the choice of cleavable site to be introduced, either a functionalized nucleoside or a modified nucleoside dimer may be first prepared, and then selectively introduced into a growing oligonucleotide fragment during the course of oligonucleotide synthesis. Selective cleavage of the dialkoxysilane may be effected by treatment with fluoride ion. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide.

In another embodiment, a non-cleavable hydroxyl linker may be converted into a cleavable linker by coupling a special phosphoramidite to the hydroxyl group prior to the phosphoramidite or H-phosphonate oligonucleotide synthesis as described in U.S. Patent Application Publication No. 2003/0186226. The cleavage of the chemical phosphorylation agent at the completion of the oligonucleotide synthesis yields an oligonucleotide bearing a phosphate group at the 3′ end. The 3′-phosphate end may be converted to a 3′ hydroxyl end by a treatment with a chemical or an enzyme, such as alkaline phosphatase, which is routinely carried out by those skilled in the art.

In another embodiment, the cleavable linking moiety may be a TOPS (two oligonucleotides per synthesis) linker (see e.g., PCT publication WO 93/20092). For example, the TOPS phosphoramidite may be used to convert a non-cleavable hydroxyl group on the solid support to a cleavable linker. A preferred embodiment of TOPS reagents is the Universal TOPS™ phosphoramidite. Conditions for Universal TOPS™ phosphoramidite preparation, coupling and cleavage are detailed, for example, in Hardy et al, Nucleic Acids Research 22(15):2998-3004 (1994). The Universal TOPS™ phosphoramidite yields a cyclic 3′ phosphate that may be removed under basic conditions, such as the extended ammonia and/or ammonia/methylamine treatment, resulting in the natural 3′ hydroxy oligonucleotide.

In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide.

In another embodiment, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al. J. of Org. Chem. 61:525-529 (1996), Kahl et al., J. of Org. Chem. 64:507-510 (1999), Kahl et al., J. of Org. Chem. 63:4870-4871 (1998), Greenberg et al., J. of Org. Chem. 59:746-753 (1994), Holmes et al., J. of Org. Chem. 62:2370-2380 (1997), and U.S. Pat. No. 5,739,386. Ortho-nitobenzyl-based linkers, such as hydroxymethyl, hydroxyethyl, and Fmoc-aminoethyl carboxylic acid linkers, may also be obtained commercially.

In another embodiment, shorter construction oligonucleotides may be synthesized and used for construction because shorter oligonucleotides should be more pure and contain fewer sequence errors than longer oligonucleotides. For example, construction oligonucleotides may be from about 30 to about 100 nucleotides, from about 30 to about 75 nucleotides, or from about 30 to about 50 oligonucleotides. In other embodiments, the construction oligonucleotides are sufficient to essentially cover the entire sequence of the synthetic polynucleotide (e.g., there are no gaps between the oligonucleotides that need to be filled in by polymerase). The oligonucleotides themselves may serve as a checking mechanism because mismatched oligonucleotides will anneal less preferentially than fully matched oligonucleotides and therefore errors containing sequences may be reduced by carefully controlling hybridization conditions.

In another embodiment, oligonucleotides may be removed from a solid support by an enzyme such as a nuclease. For example, oligonucleotides may be removed from a solid support upon exposure to one or more restriction endonucleases, including, for example, class IIs restriction enzymes. A restriction endonuclease recognition sequence may be incorporated into the immobilized oligonucleotides and the oligonucleotides may be contacted with one or more restriction endonucleases to remove the oligonucleotides from the support. In various embodiments, when using enzymatic cleavage to remove the oligonucleotides from the support, it may be desirable to contact the single stranded immobilized oligonucleotides with primers, polymerase and dNTPs to form immobilized duplexes. The duplexes may then be contacted with the enzyme (e.g., a restriction endonuclease) to remove the duplexes from the surface of the support. Methods for synthesizing a second strand on a support bound oligonucleotide and methods for enzymatic removal of support bound duplexes are described, for example, in U.S. Pat. No. 6,326,489. Alternatively, short oligonucleotides that are complementary to the restriction endonuclease recognition and/or cleavage site (e.g., but are not complementary to the entire support bound oligonucleotide) may be added to the support bound oligonucleotides under hybridization conditions to facilitate cleavage by a restriction endonuclease (see e.g., PCT Publication No. WO 04/024886).

In various embodiments, the methods disclosed herein comprise amplification of nucleic acids including, for example, construction oligonucleotides, selection oligonucleotides, subassemblies and/or polynucleotide constructs. Amplification may be carried out at one or more stages during an assembly scheme and/or may be carried out one or more times at a given stage during assembly. Amplification methods may comprise contacting a nucleic acid with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. USA. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art. In exemplary embodiments, the methods disclosed herein utilize PCR amplification.

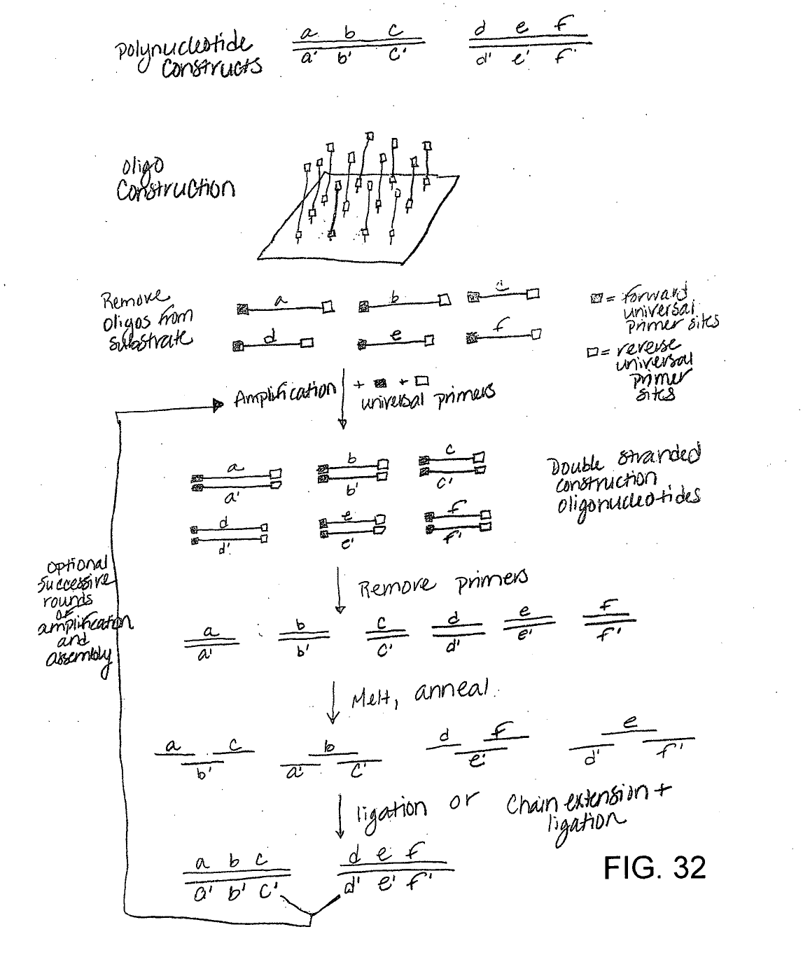

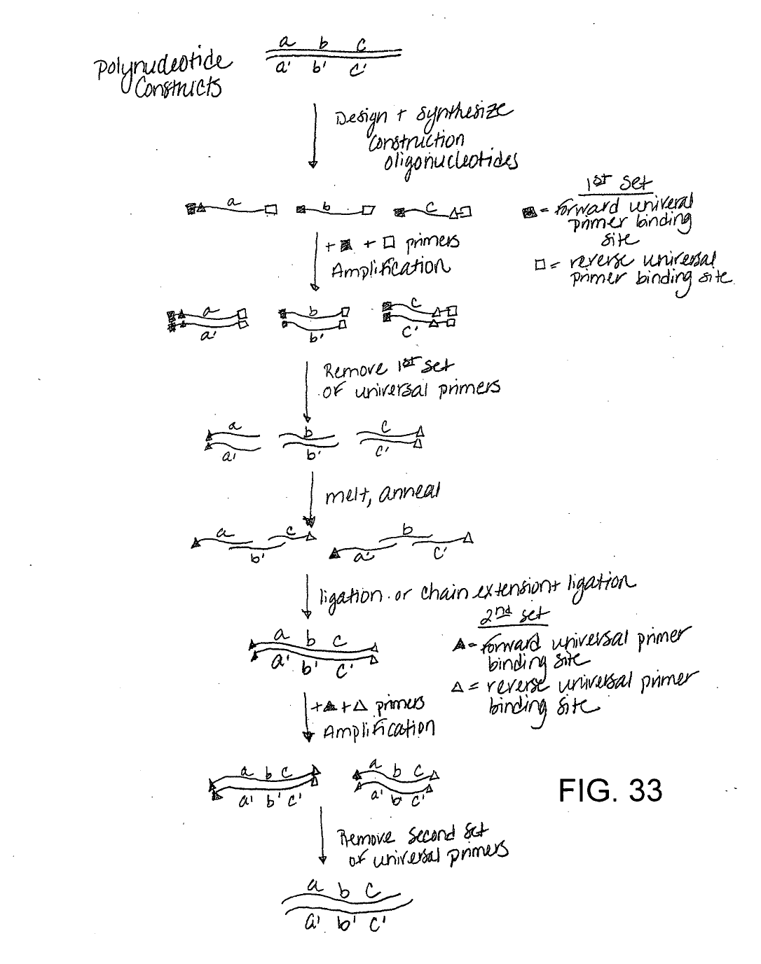

In certain embodiments, a primer set specific for a nucleic acid sequence may be used to amplify a specific nucleic acid sequence that is isolated or to amplify a specific nucleic acid sequence that is part of a pool of nucleic acid sequences. In another embodiment, a plurality of primer sets may be used to amplify a plurality of specific nucleic acid sequences that may optionally be pooled together into a single reaction mixture. In an exemplary embodiment, a set of universal primers may be used to amplify a plurality of nucleic acid sequences that may be in a single pool or separated into a plurality of pools (FIG. 32). When amplifying nucleic acids at different stages during assembly it may be desirable to utilize a different set of universal primers for each stage at which amplification is desired (FIG. 33). For example, a first set of universal primers may be used to amplify construction and/or selection oligonucleotides and a second set of universal primers may be used to amplify a subassembly or polynucleotide construct (FIG. 33). As described above, the construction oligonucleotides and/or selection oligonucleotides may be designed with primer binding sites for one or more sets of universal primers. Alternatively, primer binding sites may be added to a nucleic acid after synthesis through the use of chimeric primers that contain a region complementary to the target nucleic acid and a non-complementary region that becomes incorporated during the amplification process (see e.g., WO 99/58721).

In exemplary embodiments, primers/primer binding sites may be designed to be temporary, e.g., to permit removal of the primers/primer binding sites at a desired stage during assembly. Temporary primers may be designed so as to be removable by chemical, thermal, light based, or enzymatic cleavage. Cleavage may occur upon addition of an external factor (e.g., an enzyme, chemical, heat, light, etc.) or may occur automatically after a certain time period (e.g., after n rounds of amplification). In one embodiment, temporary primers may be removed by chemical cleavage. For example, primers having acid labile or base labile sites may be used for amplification. The amplified pool may then be exposed to acid or base to remove the primer/primer binding sites at the desired location. Alternatively, the temporary primers may be removed by exposure to heat and/or light. For example, primers having heat labile or photolabile sites may be used for amplification. The amplified pool may then be exposed to heat and/or light to remove the primer/primer binding sites at the desired location. In another embodiment, an RNA primer may be used for amplification thereby forming short stretches of RNA/DNA hybrids at the ends of the nucleic acid molecule. The primer site may then be removed by exposure to an RNase (e.g., RNase H). In various embodiments, the method for removing the primer may only cleave a single strand of the amplified duplex thereby leaving 3′ or 5′ overhangs. Such overhangs may be removed using an exonuclease to form blunt ended double stranded duplexes. For example, RecJ_{f} may be used to remove single stranded 5′ overhangs and Exonuclease I or Exonuclease T may be used to remove single stranded 3′ overhangs. Additionally, S₁ nuclease, P₁ nuclease, mung bean nuclease, and CEL I nuclease, may be used to remove single stranded regions from a nucleic acid molecule. RecJ_{f}, Exonuclease I, Exonuclease T, and mung bean nuclease are commercially available, for example, from New England Biolabs (Beverly, Mass.). S1 nuclease, P1 nuclease and CEL I nuclease are described, for example, in Vogt, V. M., Eur. J. Biochem., 33: 192-200 (1973); Fujimoto et al., Agric. Biol. Chem. 38: 777-783 (1974); Vogt, V. M., Methods Enzymol. 65: 248-255 (1980); and Yang et al., Biochemistry 39: 3533-3541 (2000).

In one embodiment, the temporary primers may be removed from a nucleic acid by chemical, thermal, or light based cleavage. Exemplary chemically cleavable internucleotide linkages for use in the methods described herein include, for example, β-cyano ether, 5′-deoxy-5′-aminocarbamate, 3′deoxy-3′-aminocarbamate, urea, 2′cyano-3′, 5′-phosphodiester, 3′-(S)-phosphorothioate, 5′-(S)-phosphorothioate, 3′-(N)-phosphoramidate, 5′-(N)-phosphoramidate, α-amino amide, vicinal diol, ribonucleoside insertion, 2′-amino-3′,5′-phosphodiester, allylic sulfoxide, ester, silyl ether, dithioacetal, 5′-thio-furmal, α-hydroxy-methyl-phosphonic bisamide, acetal, 3′-thio-furmal, methylphosphonate and phosphotriester. Internucleoside silyl groups such as trialkylsilyl ether and dialkoxysilane are cleaved by treatment with fluoride ion. Base-cleavable sites include β-cyano ether, 5′-deoxy-5′-aminocarbamate, 3′-deoxy-3′-aminocarbamate, urea, 2′-cyano-3′,5′-phosphodiester, 2′-amino-3′,5′-phosphodiester, ester and ribose. Thio-containing internucleotide bonds such as 3′-(S)-phosphorothioate and 5′-(S)-phosphorothioate are cleaved by treatment with silver nitrate or mercuric chloride. Acid cleavable sites include 3′-(N)-phosphoramidate, 5′-(N)-phosphoramidate, dithioacetal, acetal and phosphonic bisamide. An α-aminoamide internucleoside bond is cleavable by treatment with isothiocyanate, and titanium may be used to cleave a 2′-amino-3′,5′-phosphodiester-O-ortho-benzyl internucleoside bond. Vicinal diol linkages are cleavable by treatment with periodate. Thermally cleavable groups include allylic sulfoxide and cyclohexene while photo-labile linkages include nitrobenzylether and thymidine dimer. Methods synthesizing and cleaving nucleic acids containing chemically cleavable, thermally cleavable, and photo-labile groups are described for example, in U.S. Pat. No. 5,700,642.

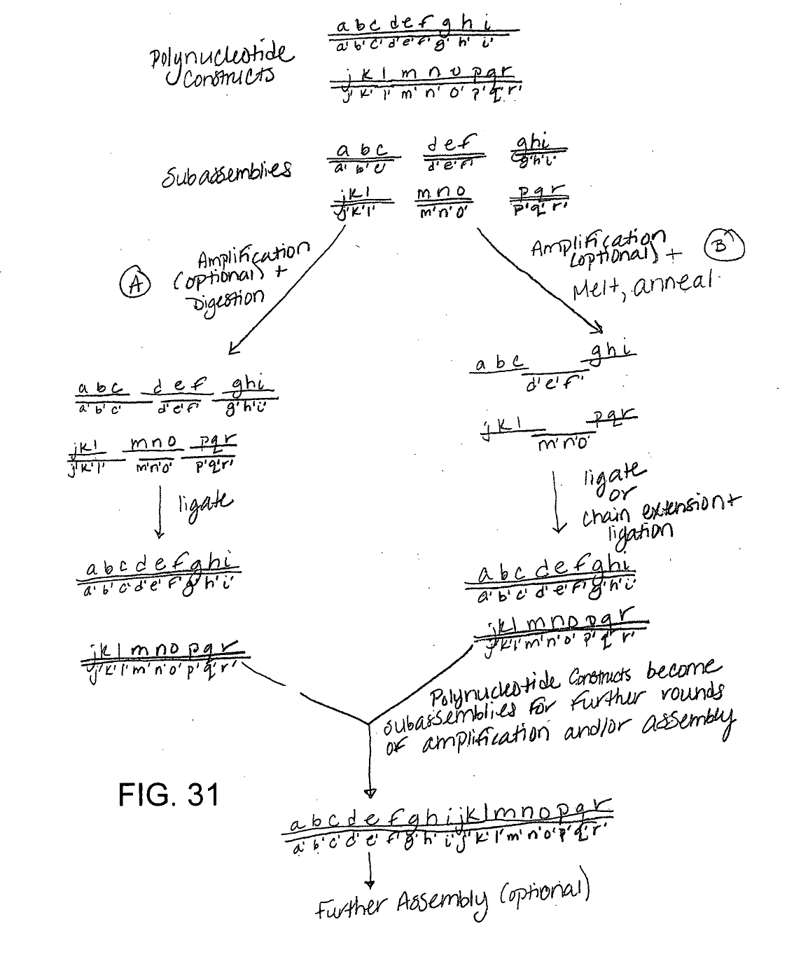

In other embodiments, temporary primers/primer binding sites may be removed using enzymatic cleavage. For example, primers/primer binding sites may be designed to include a restriction endonuclease cleavage site. After amplification, the pool of nucleic acids may be contacted with one or more endonucleases to produce double stranded breaks thereby removing the primers/primer binding sites. In certain embodiments, the forward and reverse primers may be removed by the same or different restriction endonucleases. Any type of restriction endonuclease may be used to remove the primers/primer binding sites from nucleic acid sequences. A wide variety of restriction endonucleases having specific binding and/or cleavage sites are commercially available, for example, from New England Biolabs (Beverly, Mass.). In various embodiments, restriction endonucleases that produce 3′ overhangs, 5′ overhangs or blunt ends may be used. When using a restriction endonuclease that produces an overhang, an exonuclease (e.g., RecJ_{f}, Exonuclease I, Exonuclease T, S₁ nuclease, P₁ nuclease, mung bean nuclease, CEL I nuclease, etc.) may be used to produce blunt ends. Alternatively, the sticky ends formed by the specific restriction endonuclease may be used to facilitate assembly of subassemblies in a desired arrangement (see e.g., FIG. 31A). In an exemplary embodiment, a primer/primer binding site that contains a binding and/or cleavage site for a type IIS restriction endonuclease may be used to remove the temporary primer.

Primers suitable for use in the amplification methods disclosed herein may be designed with the aid of a computer program, such as, for example, DNAWorks (supra), Gene2Oligo (supra), or CAD-PAM software described herein. Typically primers are from about 5 to about 500, about 10 to about 100, about 10 to about 50, or about 10 to about 30 nucleotides in length. In exemplary embodiments, a set of primers or a plurality of sets of primers may be designed so as to have substantially similar melting temperatures to facilitate manipulation of a complex reaction mixture. The melting temperature may be influenced, for example, by primer length and nucleotide composition.

In certain embodiments, it may be desirable to utilize a primer comprising one or more modifications such as a cap (e.g., to prevent exonuclease cleavage), a linking moiety (such as those described above to facilitate immobilization of an oligonucleotide onto a substrate), or a label (e.g., to facilitate detection, isolation and/or immobilization of a nucleic acid construct). Suitable modifications include, for example, various enzymes, prosthetic groups, luminescent markers, bioluminescent markers, fluorescent markers (e.g., fluorescein), radiolabels (e.g., ³²P, ³⁵S, etc.), biotin, polypeptide epitopes, etc. Based on the disclosure herein, one of skill in the art will be able to select an appropriate primer modification for a given application.

In one embodiment, the present invention provides methods for sequence optimization and oligonucleotides design. In one aspect, the invention provides a method for designing a set of end-overlapping oligonucleotides for each gene that alternates on both the plus and minus strands. In another aspect, the oligonucleotides together cover an entire sequence to be synthesized. In another aspect, oligonucleotide design is aided by a computer program. In another aspect, protein-coding sequences are optimized by a computer program, i.e., the CAD-PAM program described herein.

Embodiments of the present invention are directed to oligonucleotide sequences (i.e., construction oligonucleotide sequences and selection oligonucleotide sequences) having one or more amplification sequences or amplification sites. As used herein, the term “amplification site” is intended to include, but is not limited to, a nucleic acid sequence located at the 5′ and/or 3′ end of the oligonucleotide sequences of the present invention which hybridizes a complementary nucleic acid sequence. In one aspect of the invention, an amplification site is removed from the oligonucleotide after amplification. In another aspect of the invention, an amplification site includes one or more restriction endonuclease recognition sequences recognized by one or more restriction enzymes. In another aspect, an amplification site is heat labile and/or photo labile and is cleavable by heat or light, respectively. In yet another aspect, an amplification site is a ribonucleic acid sequence cleavable by RNase.

As used herein, the term “restriction endonuclease recognition site” is intended to include, but is not limited to, a particular nucleic acid sequence to which one or more restriction enzymes bind, resulting in cleavage of a DNA molecule either at the restriction endonuclease recognition sequence itself, or at a sequence distal to the restriction endonuclease recognition sequence. Restriction enzymes include, but are not limited to, type I enzymes, type II enzymes, type IIS enzymes, type III enzymes and type IV enzymes. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts et al. (2005) Nuc. Acids Res. 33:D230, incorporated herein by reference in its entirety for all purposes).

In certain aspects, primers of the present invention include one or more restriction endonuclease recognition sites that enable type IIS enzymes to cleave the nucleic acid several base pairs 3′ to the restriction endonuclease recognition sequence. As used herein, the term “type IIS” refers to a restriction enzyme that cuts at a site remote from its recognition sequence. Type IIS enzymes are known to cut at a distances from their recognition sites ranging from 0 to 20 base pairs. Examples of Type IIs endonucleases include, for example, enzymes that produce a 3′ overhang, such as, for example, Bsr I, Bsm I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632 I, Eco31 I, Esp3 I, Aar I; and enzymes that produce a blunt end, such as, for example, Mly I and Btr I. Type-IIs endonucleases are commercially available and are well known in the art (New England Biolabs, Beverly, Mass.). Information about the recognition sites, cut sites and conditions for digestion using type IIs endonucleases may be found, for example, on the world wide web at neb.com/nebecomm/enzymefindersearch bytypells.asp). Restriction endonuclease sequences and restriction enzymes are well known in the art and restriction enzymes are commercially available (New England Biolabs, Beverly, Mass.).

In certain embodiments, primers are provided having a detectable label. Detectable labels include, but are not limited to, various enzymes, prosthetic groups, luminescent markers, bioluminescent markers, fluorescent markers, and the like. Examples of suitable luminescent and bioluminescent markers include, but are not limited to, biotin, luciferase (e.g., bacterial, firefly, click beetle and the like), luciferin, aequorin and the like. Examples of suitable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of suitable enzyme systems having visually detectable signals include, but are not limited to, galactosidases, glucorinidases, phosphatases, peroxidases, cholinesterases and the like. Detectable labels also include, but are not limited to, radiolabeled nucleic acids e.g., labeled with ³²P, ³⁵S, and the like, either directly or indirectly. Alternatively, compounds can be enzymatically labeled with, for example, horseradish peroxidase, alkaline phosphatase, or luciferase, and the enzymatic label detected by determination of conversion of an appropriate substrate to product.

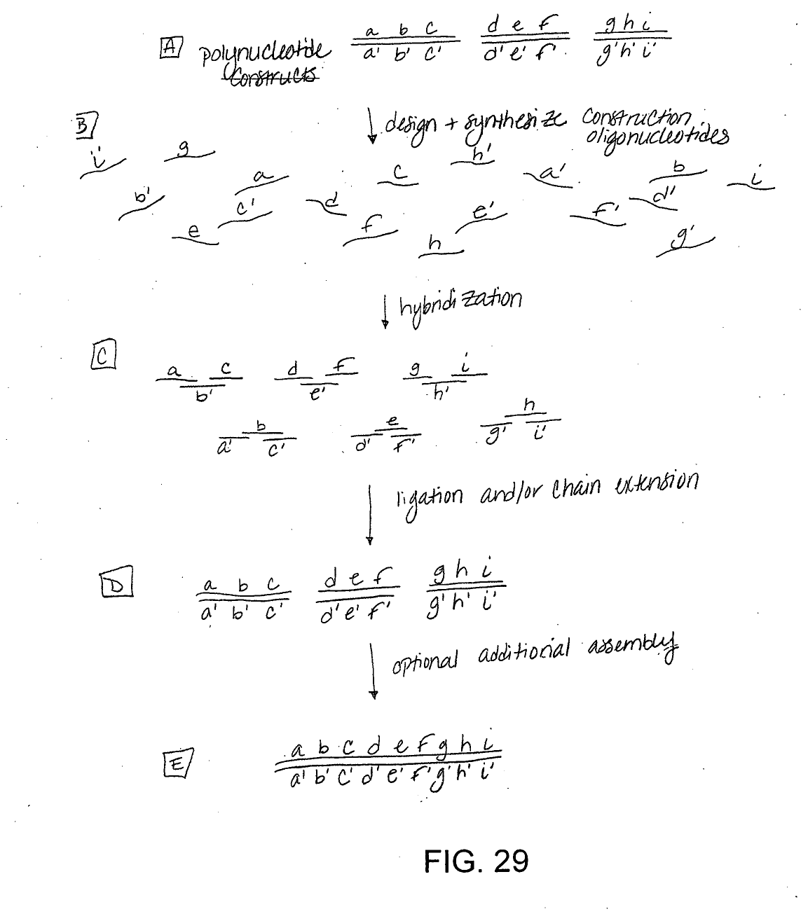

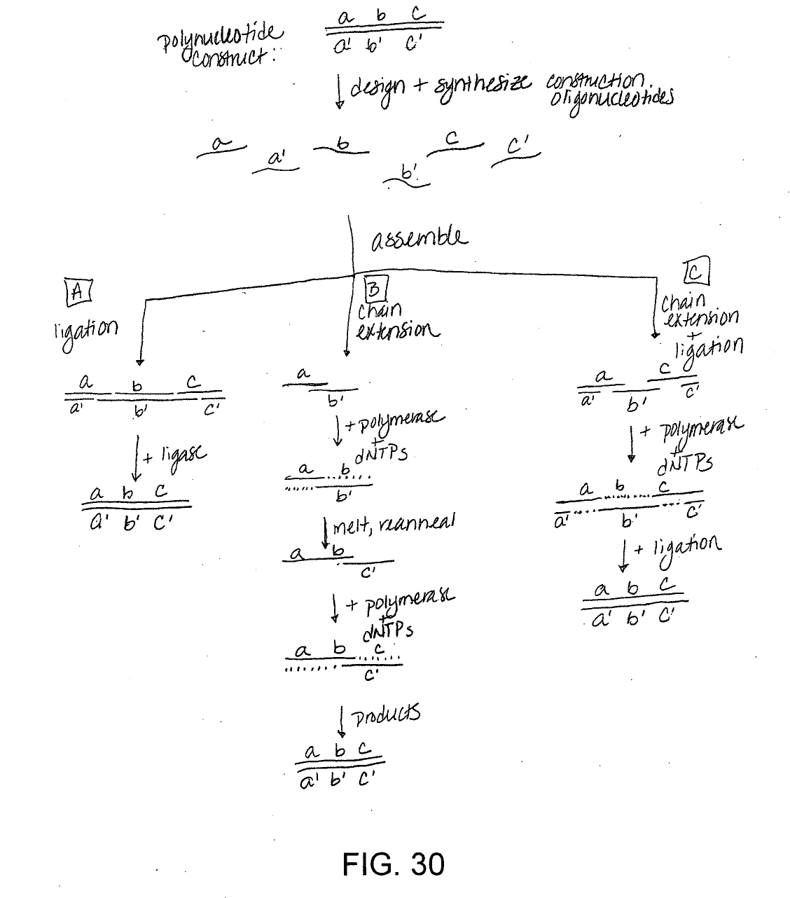

Certain embodiments of the present invention are directed to methods of synthesizing nucleic acid sequences and very long sequences (e.g., genes, gene sets, genomes and the like) in which sets of overlapping oligonucleotides and/or amplification primers are mixed under conditions that favor sequence-specific hybridizations and the oligonucleotides are extended by one or more polymerases using the hybridizing strand as a template (i.e., polymerase assembly multiplexing (PAM) described in Tian et al. (2004) Nature 432:1050, incorporated by reference herein in its entirety for all purposes). Multiplex assembly is illustrated in FIGS. 29-33. In one aspect, double stranded extension products are denatured for further rounds of the above process until full-length double-stranded DNA molecules are synthesized and amplified. Multiplex gene syntheses may be performed either in solution or on a support (e.g., as part of an array) as described herein. Successful use of the methods described herein have recently been confirmed by Zhou et al. (2004) Nucleic Acids Res. 32:5409 and Richmond et al. (2004) Nucleic Acids Res. 32:5011 (incorporated by reference herein in their entirety for all purposes).

In addition to polymerase assembly multiplexing, a variety of methods are suitable for obtaining large double-stranded nucleic acid sequences using the oligonucleotides and methods of the invention described herein. For example, PCR based assembly methods (including PAM or polymerase assembly multiplexing) and ligation based assembly methods (e.g., joining of polynucleotide segments having cohesive ends). In an exemplary embodiment, a plurality of polynucleotide constructs may be assembled in a single reaction mixture. In other embodiments, hierarchical based assembly methods may be used, for example, when synthesizing a large number of polynucleotide constructs, when synthesizing a polynucleotide construct that contains a region of internal homology, or when synthesizing two or more polynucleotide constructs that are highly homologous or contain regions of homology.

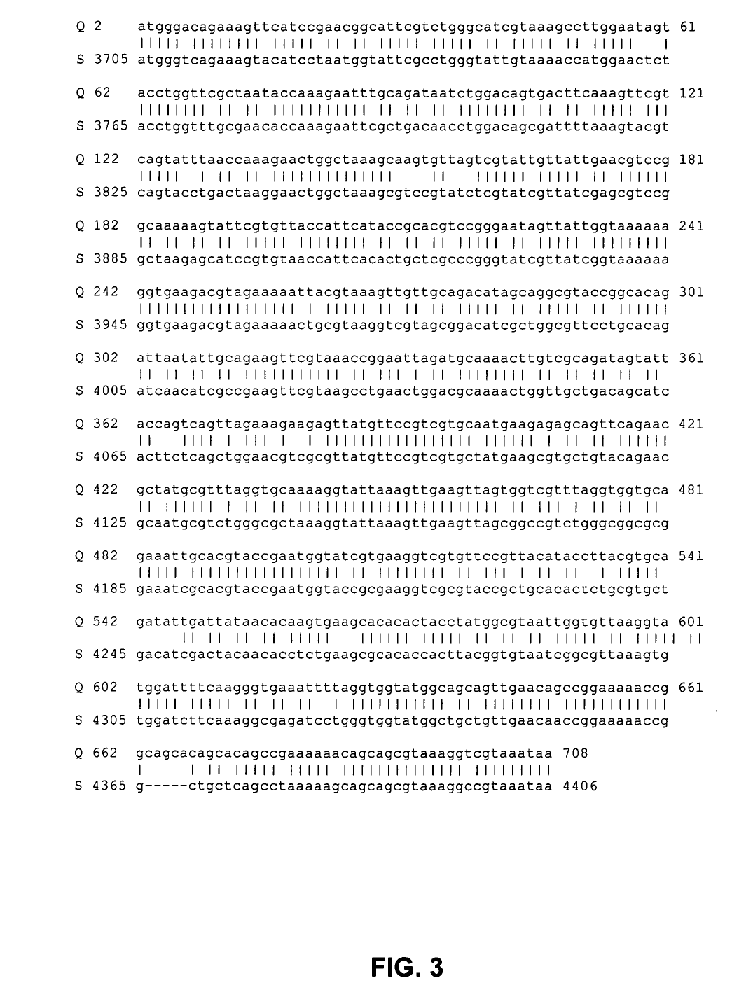

In one embodiment, assembly PCR may be used in accordance with the methods described herein. Assembly PCR uses polymerase-mediated chain extension in combination with at least two polynucleotides having complementary ends which can anneal such that at least one of the polynucleotides has a free 3′-hydroxyl capable of polynucleotide chain elongation by a polymerase (e.g., a thermostable polymerase (e.g., Taq polymerase, VENT™ polymerase (New England Biolabs), TthI polymerase (Perkin-Elmer) and the like). Overlapping oligonucleotides may be mixed in a standard PCR reaction containing dNTPs, a polymerase, and buffer. The overlapping ends of the oligonucleotides, upon annealing, create regions of double-stranded nucleic acid sequences that serve as primers for the elongation by polymerase in a PCR reaction. Products of the elongation reaction serve as substrates for formation of a longer double-strand nucleic acid sequences, eventually resulting in the synthesis of full-length target sequence (see e.g., FIG. 3B). The PCR conditions may be optimized to increase the yield of the target long DNA sequence.

In certain embodiments, the target sequence may be obtained in a single step by mixing together all of the overlapping oligonucleotides needed to form the polynucleotide construct of interest. Alternatively, a series of PCR reactions may be performed in parallel or serially, such that larger polynucleotide constructs may be assembled from a series of separate PCR reactions whose products are mixed and subjected to a second round of PCR. Moreover, if the self-priming PCR fails to give a full-sized product from a single reaction, the assembly may be rescued by separately PCR-amplifying pairs of overlapping oligonucleotides, or smaller sections of the target nucleic acid sequence, or by conventional filling-in and ligation methods.

Methods for performing assembly PCR are described, for example, in Kodumal et al. (2004) Proc. Natl. Acad. Sci. USA. 101:15573; Stemmer et al. (1995) Gene 164:49; Dillon et al. (1990) BioTechniques 9:298; Hayashi et al. (1994) BioTechniques 17:310; Chen et al. (1994) J. Am. Chem. Soc. 116:8799; Prodromou et al. (1992) Protein Eng. 5:827; U.S. Pat. Nos. 5,928,905 and 5,834,252; and U.S. Patent Application Publication Nos. 2003/0068643 and 2003/0186226.

In an exemplary embodiment, polymerase assembly multiplexing (PAM) may be used to assemble polynucleotide constructs in accordance with the methods described herein (see e.g., Tian et al. (2004) Nature 432:1050; Zhou et al. (2004) Nucleic Acids Res. 32:5409; and Richmond et al. (2004) Nucleic Acids Res. 32:5011). Polymerase assembly multiplexing involves mixing sets of overlapping oligonucleotides and/or amplification primers under conditions that favor sequence-specific hybridization and chain extension by polymerase using the hybridizing strand as a template. The double stranded extension products may optionally be denatured and used for further rounds of assembly until a desired polynucleotide construct has been synthesized.

In various embodiments, methods for assembling polynucleotide constructs in accordance with the methods described herein include, for example, ligation of preformed duplexes (see e.g., Scarpulla et al., Anal. Biochem. 121: 356-365 (1982); Gupta et al., Proc. Natl. Acad. Sci. USA 60: 1338-1344 (1968)), the Fok I method (see e.g., Mandecki and Bolling, Gene 68: 101-107 (1988)), dual asymmetrical PCR (DA-PCR) (see e.g., Stemmer et al., Gene 164: 49-53 (1995); Sandhu et al., Biotechniques 12: 14-16 (1992); Smith et al., Proc. Natl. Acad. Sci. USA 100: 15440-15445 (2003)), overlap extension PCR (OE-PCR) (see e.g., Mehta and Singh, Biotechniques 26: 1082-1086 (1999)), DA-PCR/OE-PCR combination (see e.g., Young and Dong, Nucleic Acids Res. 32: e59 (2004)).

In another embodiment, a combinatorial assembly strategy may be used for assembly of polynucleotides (see e.g., U.S. Pat. Nos. 6,670,127, 6,521,427 and 6,521,427). Briefly, oligonucleotides may be jointly co-annealed by temperature-based slow annealing followed by ligation chain reaction steps using a new oligonucleotide addition with each step. The first oligonucleotide in the chain is attached to a support. The second, overlapping oligonucleotide from the opposite strand is added, annealed and ligated. The third, overlapping oligonucleotide is added, annealed and ligated, and so forth. This procedure is replicated until all oligonucleotides of interest are annealed and ligated. This procedure can be carried out for long sequences using an automated device. The double-stranded nucleic acid sequence is then removed from the solid support.

In certain embodiments, hierarchical assembly strategies may be used in accordance with the methods disclosed herein. Hierarchical assembly strategies include various methods for controlled mixing of various components of a reaction mixture so as to control the assembly in a staged or stepwise manner (see e.g., U.S. Pat. No. 6,586,211; U.S. Patent Application Publication No. 2004/0166567; PCT Publication No. WO 02/095073; Zhou et al. (2004) Nucleic Acids Res. 32:5409). For example, a plurality of assembly reactions may be conducted in separate pools. Products from these assemblies may then be mixed to together to form even larger assembled products, etc. Alternatively, hierarchical assembly strategies may involve a single reaction mixture that permits external control by varying the reactive species in the mixture. For example, oligonucleotides attached to a solid support via a photolabile linker may be released from the support in a highly specific and controlled manner that can be used to facilitate ordered assembly (e.g., oligonucleotides may be removed from a single addressable location on a solid support in a controlled fashion). A first set of construction oligonucleotides may be released from the support and subjected to assembly. Subsequently a second set of construction oligonucleotides may be released from the support and assembled, etc. In one embodiment, positive and negative strands of construction oligonucleotides may be synthesized on different locations or on different supports. The positive and negative strands may then be released from the chips into separate pools and mixed in a controlled fashion. In another embodiment, hierarchical assembly may be controlled by proximity of construction oligonucleotides on a solid support. For example, two construction oligonucleotides having complementary regions may be synthesized in close proximity to each other. Upon release from the solid support, oligonucleotides located in close proximity to each other will favorably interact due to the higher local concentrations of the oligonucleotides. In an exemplary embodiment, two or more construction oligonucleotides may be synthesized at the same location on a solid support thereby facilitating their interaction (see e.g., U.S. Patent Publication No. 2004/0101894). In yet another embodiment, microfluidic systems may be employed to control the reaction mixture and facilitate the assembly process. For example, oligonucleotides may be synthesized in a flow cell containing channels such that the features of the array are aligned in linear rows which are physically separated from one another thus separate, linear channels in which fluids may flow. Oligonucleotides in a given channel may hybridize with interact with other oligonucleotides in the same channel but will not be exposed to oligonucleotides from other channels. When adjoining oligonucleotide sequences are synthesized in the same channel, they can hybridize to one another after cleavage from the array to form “sub-assemblies”. Various sub-assemblies may then be contacted with other sub-assemblies in order to hybridize larger nucleic acid sequences. Ligases and/or polymerases may be added as needed to fill in and join gaps in the nucleic acid sequences.

In yet another embodiment, hierarchical assembly may be carried out using restriction endonucleases to form cohesive ends that may be joined together in a desired order. The construction oligonucleotides may be designed and synthesized to contain recognition and cleavage sites for one or more restriction endonucleases at sites that would facilitate joining in a specified order. After forming DNA duplexes, the pool of oligonucleotides may be contacted with one or more restriction endonucleases to form the cohesive ends. The pool is then exposed to hybridization and ligation conditions to join the duplexes together. The order of joining will be determined by hybridization of the complementary cohesive ends. The restriction endonucleases may be added in a staggered fashion so as to form only a subset of cohesive ends at a time. These ends may then be joined together followed by another round of endonuclease digestion, hybridization, ligation, etc. In an exemplary embodiment, a type IIS endonuclease recognition site may be incorporated into the termini of the construction oligonucleotides to permit cleavage by a type IIS restriction endonuclease.

Mutations incurred during oligonucleotide synthesis are a major source of errors in assembled DNA molecules, and are costly and difficult to eradicate (Cello et al. (2002) Science 297:1016; Smith et al. (2003) Proc. Natl. Acad. Sci. USA 100:15440; incorporated by reference herein in their entirety for all purposes). Accordingly, in various embodiments, various error reduction methods may be used to remove errors in construction oligonucleotides, subassemblies and/or polynucleotide constructs. Error correction methods may include for example, error filtration, error neutralization and error correction methods as described below.

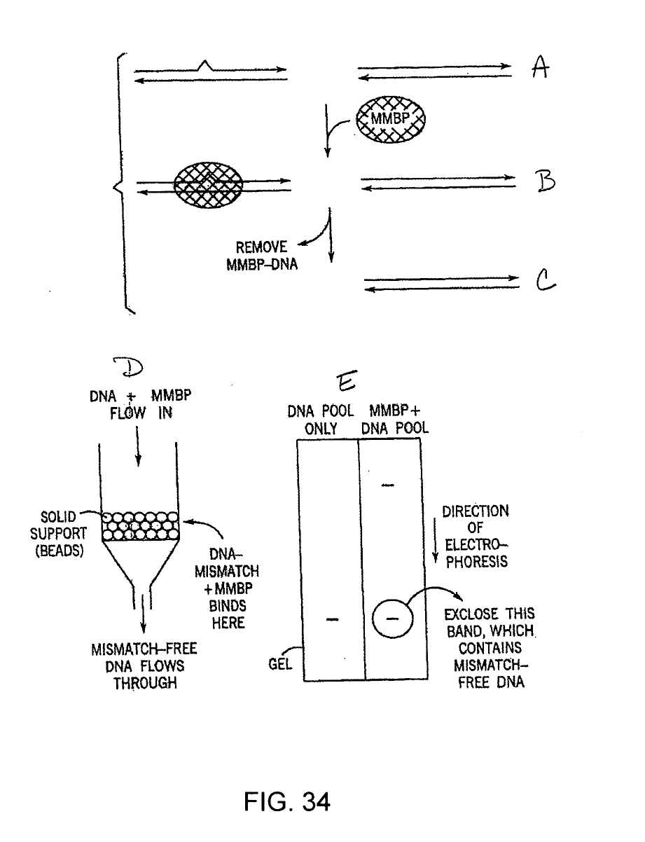

Proteins involved in mismatch repair, such as mismatch binding proteins, can be used to select synthetic oligonucleotides having the correct nucleotide sequence (FIGS. 34-36). Mismatch repair proteins bind to a variety of DNA mismatches, deletions and insertions (Carr et al. (2004) Nucleic Acids Res. 32:e162). Accordingly, mismatch binding proteins can be used to bind to synthetic oligonucleotide sequences which have errors. Double-stranded oligonucleotide sequences (e.g., hybridized construction oligonucleotides, hybridized selection oligonucleotides and/or a construction oligonucleotide hybridized to a selection oligonucleotide) that are error free may then be separated from double-stranded oligonucleotides sequences bound to mismatch binding proteins. Thus, error-free oligonucleotides sequences can be effectively separated from oligonucleotide sequences that contain errors.

The term “DNA repair” refers to a process wherein sequence errors in a nucleic acid (DNA:DNA duplexes, DNA:RNA and, for purposes herein, also RNA:RNA duplexes) are recognized by a nuclease that excises the damaged or mutated region from the nucleic acid; and then further enzymes or enzymatic activities synthesize a replacement portion of a strand(s) to produce the correct sequence.

The term “DNA repair enzyme” refers to one or more enzymes that correct errors in nucleic acid structure and sequence, i.e., recognizes, binds and corrects abnormal base-pairing in a nucleic acid duplex. Examples of DNA repair enzymes include, but are not limited to, proteins such as mutH, mutL, mutM, mutS, mutY, dam, thymidine DNA glycosylase (TDG), uracil DNA glycosylase, AlkA, MLH1, MSH2, MSH3, MSH6, Exonuclease I, T4 endonuclease V, Exonuclease V, RecJ exonuclease, FEN1 (RAD27), dnaQ (mutD), polC (dnaE), or combinations thereof, as well as homologs, orthologs, paralogs, variants, or fragments of the forgoing. Enzymatic systems capable of recognition and correction of base pairing errors within the DNA helix have been demonstrated in bacteria, fungi and mammalian cells and the like.

As used herein the terms “mismatch binding agent” or “MMBA” refer to an agent that binds to a double stranded nucleic acid molecule that contains a mismatch. The agent may be chemical or proteinaceous. In certain embodiments, an MMBA is a mismatch binding protein (MMBP) such as, for example, Fok I, MutS, T7 endonuclease, a DNA repair enzyme as described herein, a mutant DNA repair enzyme as described in U.S. Patent Publication No. 2004/0014083, or fragments or fusions thereof. Mismatches that may be recognized by an MMBA include, for example, one or more nucleotide insertions or deletions, or improper base pairing, such as A:A, A:C, A:G, C:C, C:T, G:G, G:T, T:T, C:U, G:U, T:U, U:U, 5-formyluracil (fU):G, 7,8-dihydro-8-oxo-guanine (8-oxoG):C, 8-oxoG:A or the complements thereof.

As used herein, the terms “MLH1” and “PMS1” (PMS2 in humans) refers to the components of the eukaryotic mutL-related protein complex, e.g., MLH1-PMS1, that interacts with MSH2-containing complexes bound to mispaired bases. Exemplary MLH1 proteins include, for example, polypeptides encoded by nucleic acids having the following GenBank accession Nos. A1389544 (D. melanogaster), A1387992 (D. melanogaster), AF068257 (D. melanogaster), U80054 (Rattus norvegicus) and U07187 (S. cerevisiae), as well as homologs, orthologs, paralogs, variants, or fragments thereof.

As used herein, the term “MSH2” refers to a component of the eukaryotic DNA repair complex that recognizes base mismatches and insertion or deletion of up to 12 bases. MSH2 forms heterodimers with MSH3 or MSH6. MSH2 proteins include, for example, polypeptides encoded by nucleic acids having the following GenBank accession Nos.: AF109243 (A. thaliana), AF030634 (Neurospora crassa), AF002706 (A. thaliana), AF026549 (A. thaliana), L47582 (H. sapiens), L47583 (H. sapiens), L47581 (H. sapiens) and M84170 (S. cerevisiae) and homologs, orthologs, paralogs, variants, or fragments thereof. MSH3 proteins include, for example, polypeptides encoded by the nucleic acids having GenBank accession Nos.: J04810 (H. sapiens) and M96250 (Saccharomyces cerevisiae) and homologs, orthologs, paralogs, variants, or fragments thereof. MSH6 proteins include, for example, polypeptide encoded by nucleic acids having the following GenBank accession Nos.: U54777 (H. sapiens) and AF031087 (M. musculus) and homologs, orthologs, paralogs, variants, or fragments thereof.

As used herein, the term “mutH” refers to a latent endonuclease that incises the unmethylated strand of a hemimethylated DNA, or makes a double strand cleavage on unmethylated DNA, 5′ to the G of d(GATC) sequences. The term is meant to include prokaryotic mutH (e.g., Welsh et al., 262 J. Biol. Chem. 15624 (1987)) as well as homologs, orthologs, paralogs, variants, or fragments thereof.

As used herein, the term “mutHLS” refers to a complex between mutH, mutL, and mutS proteins (or homologs, orthologs, paralogs, variants, or fragments thereof).

As used herein, the term “mutL” refers to a protein that couples abnormal base-pairing recognition by mutS to mutH incision at the 5′-GATC-3′ sequences in an ATP-dependent manner. The term is meant to encompass prokaryotic mutL proteins as well as homologs, orthologs, paralogs, variants, or fragments thereof. MutL proteins include, for example, polypeptides encoded by nucleic acids having the following GenBank accession Nos. AF170912 (C. crescentus), AI518690 (D. melanogaster), A1456947 (D. melanogaster), A1389544 (D. melanogaster), A1387992 (D. melanogaster), AI292490 (D. melanogaster), AF068271 (D. melanogaster), AF068257 (D. melanogaster), U50453 (T. aquaticus), U27343 (B. subtilis), U71053 (U71053 (T. maritima), U71052 (A. pyrophilus), U13696 (H. sapiens), U13695 (H. sapiens), M29687 (S. typhimurium), M63655 (E. coli) and L19346 (E. coli). MutL homologs include, for example, eukaryotic MLH1, MLH2, PMS1, and PMS2 proteins (see e.g., U.S. Pat. Nos. 5,858,754 and 6,333,153, incorporated herein by reference in their entirety).

As used herein, the term “mutS” refers to a DNA-mismatch binding protein that recognizes and binds to a variety of mispaired bases and small (1-5 bases) single-stranded loops. The term is meant to encompass prokaryotic mutS proteins as well as homologs, orthologs, paralogs, variants, or fragments thereof. The term also encompasses homo- and hetero-dimmers and multimers of various mutS proteins. MutS proteins include, for example, polypeptides encoded by nucleic acids having the following GenBank accession Nos. AF146227 (M. musculus), AF193018 (A. thaliana), AF144608 (V. parahaemolyticus), AF034759 (H. sapiens), AF104243 (H. sapiens), AF007553 (T. aquaticus caldophilus), AF109905 (M. musculus), AF070079 (H. sapiens), AF070071 (H. sapiens), AH006902 (H. sapiens), AF048991 (H. sapiens), AF048986 (H. sapiens), U33117 (T. aquaticus), U16152 (Y. enterocolitica), AF000945 (V. cholarae), U698873 (E. coli), AF003252 (H. influenzae strain b (Eagan)), AF003005 (A. thaliana), AF002706 (A. thaliana), L10319 (M. musculus), D63810 (T. thermophilus), U27343 (B. subtilis), U71155 (T. maritima), U71154 (A. pyrophilus), U16303 (S. typhimurium), U21011 (M. musculus), M84170 (S. cerevisiae), M84169 (S. cerevisiae), M18965 (S. typhimurium) and M63007 (A. vinelandii). MutS homologs include, for example, eukaryotic MSH2, MSH3, MSH4, MSH5, and MSH6 proteins (see e.g., U.S. Pat. Nos. 5,858,754 and 6,333,153).

In one aspect, the invention provides methods for increasing the fidelity of a polynucleotide pool by removing polynucleotide copies that contain errors via hybridization to one or more selection oligonucleotides. This type of error filtration process may be carried out on oligonucleotides at any stage of assembly, for example, construction oligonucleotides, subassemblies, and in some cases larger polynucleotide constructs. Error filtration using selection oligonucleotides may be conducted before and/or after amplification of the polynucleotide pool. In an exemplary embodiment, error filtration using selective oligonucleotides is used to increase the fidelity of the pool of construction oligonucleotides before and/or after amplification. An illustrative embodiment of error filtration through hybridization to selection oligonucleotides is shown in FIG. 32. A pool of construction oligonucleotides has been amplified using universal primers. Some of the construction oligonucleotides contain errors which are represented by a bulge in the strand. These errors may have arisen from the initial synthesis of the construction oligonucleotides or may have been introduced during the amplification process. The pool of construction oligonucleotides is then denatured to produce single strands and contacted with at least one pool of selection oligonucleotides under hybridization conditions. The pool of selection oligonucleotides comprises one or more selection oligonucleotides complementary to each of the construction oligonucleotides in the pool (e.g., the pool of selection oligos is at least as large as the pool of construction oligonucleotides, and in some cases may comprise, e.g., twice as many different oligonucleotides as compared to the pool of construction oligonucleotides). Copies of construction oligonucleotides that do not perfectly pair with a selection oligonucleotide (e.g., there is a mismatch) will not hybridize as tightly as perfectly matched copies and can be removed from the pool by controlling the stringency of the hybridization conditions. After removal of the oligonucleotides containing mismatches, the perfectly matched copies of the construction oligonucleotides may be removed by increasing the stringency conditions to elute them off of the selection oligonucleotides. In an exemplary embodiment, the selection oligonucleotides may be end immobilized (e.g., via chemical linkage, biotin/streptavidin, etc.) to facilitate removal of oligonucleotide copies containing errors. For example, the selection oligonucleotides may be immobilized on beads before or after hybridization to the pool of construction oligonucleotides. The beads may then be pelleted, or loaded onto a column, and exposed to different stringency conditions to remove copies of construction oligonucleotides containing a mismatch with the selection oligonucleotide. In certain embodiments, it may be desirable to submit the oligonucleotides to iterative rounds of amplification and error filtration through hybridization to a pool of selection oligonucleotides thereby increasing the number of copies of oligonucleotides in the pool while maintaining, or preferably increasing, the fidelity of the pool (e.g., increasing the number of error free copies in the pool).

It should be noted that in some instances, the mismatch between the construction and selection oligonucleotides will arise from a sequence error in the selection oligonucleotide thereby removing an error free construction oligonucleotide from the pool. However, the net effect will still be increased fidelity of the construction oligonucleotide pool.

FIG. 34 illustrates another exemplary method for error filtration that may be used to increase the fidelity of a pool of double stranded construction oligonucleotides, subassemblies and/or polynucleotide constructs. An error in a single strand of DNA causes a mismatch in a DNA duplex. A mismatch binding protein (MMBP), such as a dimer of MutS, binds to this site on the DNA. As shown in FIG. 34A, a pool of DNA duplexes contains some duplexes with mismatches (left) and some which are error-free (right). The 3′-terminus of each DNA strand is indicated by an arrowhead. An error giving rise to a mismatch is shown as a raised triangular bump on the top left strand. As shown in FIG. 34B, a MMBP may be added which binds selectively to the site of the mismatch. The MMBP-bound DNA duplex may then be removed, leaving behind a pool which is dramatically enriched for error-free duplexes (FIG. 34C). In one embodiment, the DNA-bound protein provides a means to separate the error-containing DNA from the error-free copies (FIG. 34D). The protein-DNA complexes can be captured by affinity of the protein for a solid support functionalized, for example, with a specific antibody, immobilized nickel ions (protein is produced as a his-tag fusion), streptavidin (protein has been modified by the covalent addition of biotin) or other such mechanisms as are common to the art of protein purification. Alternatively, the protein-DNA complex is separated from the pool of error-free DNA sequences by a difference in mobility, for example, using a size-exclusion column chromatography or by electrophoresis (FIG. 34E). In this example, the electrophoretic mobility in a gel is altered upon MMBP binding: in the absence of MMBP all duplexes migrate together, but in the presence of MMBP, mismatch duplexes are retarded (upper band). The mismatch-free band (lower) is then excised and extracted.

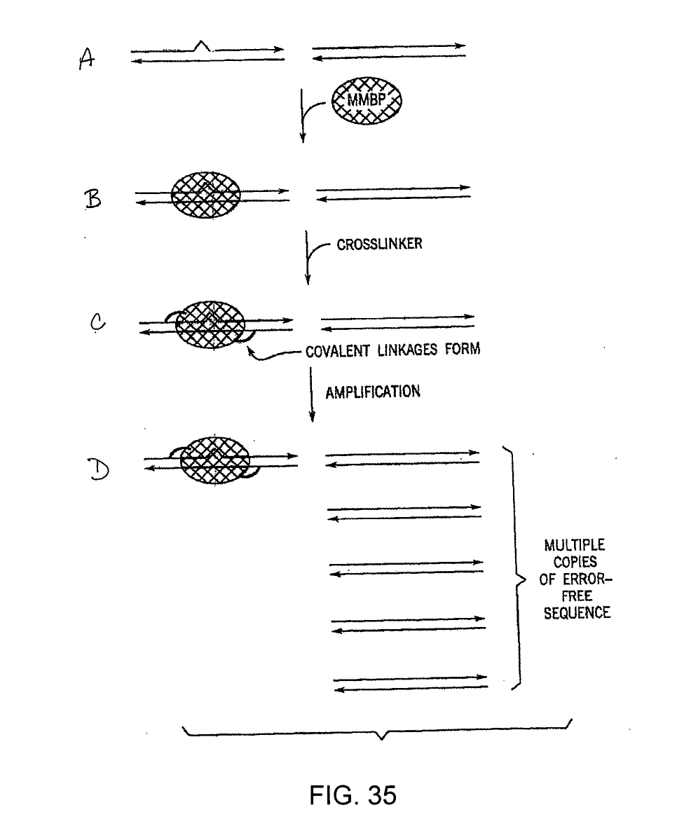

FIG. 35 illustrates an exemplary method for neutralizing sequence errors using a mismatch binding agent. This type of error reduction method may be useful to increase the fidelity of a pool of double stranded construction oligonucleotides, subassemblies and/or polynucleotide constructs. In this embodiment, the error-containing DNA sequence is not removed from the pool of DNA products. Rather, it becomes irreversibly complexed with a mismatch recognition protein by the action of a chemical crosslinking agent (for example, dimethyl suberimidate, DMS), or of another protein (such as MutL). The pool of DNA sequences is then amplified (such as by the polymerase chain reaction, PCR), but those containing errors are blocked from amplification, and quickly become outnumbered by the increasing error-free sequences. FIG. 35A illustrates an exemplary pool of DNA duplexes containing some duplexes with mismatches (left) and some which are error-free (right). A MMBP may be used to bind selectively to the DNA duplexes containing mismatches (FIG. 35B). The MMBP may be irreversibly attached at the site of the mismatch upon application of a crosslinking agent (FIG. 35C). In the presence of the covalently linked MMBP, amplification of the pool of DNA duplexes produces more copies of the error-free duplexes (FIG. 35D). The MMBP-mismatch DNA complex is unable to participate in amplification because the bound protein prevents the two strands of the duplex from dissociating. For long DNA duplexes, the regions outside the MMBP-bound site may be able to partially dissociate and participate in partial amplification of those (error-free) regions.

As increasingly longer sequences of DNA are generated, the fraction of sequences which are completely error-free diminishes. At some length, it becomes likely that there will be no molecule in the entire pool which contains a completely correct sequence. Thus, for the generation of extremely long segments of DNA, it can be useful to produce smaller units first which can be subjected to the above error control approaches. Then these segments can be combined to yield the larger full length product. However, if errors in these extremely long sequences can be corrected locally, without removing or neutralizing the entire long DNA duplex, then the more complex stepwise assembly process can be avoided.

Many biological DNA repair mechanisms rely on recognizing the site of a mutation (error) and then using a template strand (most likely error-free) to replace the incorrect sequence. In the de novo production of DNA sequences, this process is complicated by the difficulty of determining which strand contains the error and which should be used as the template. Solutions to this problem rely on using the pool of other sequences in the mixture to provide the template for correction. These methods can be very robust: even if every strand of DNA contains one or more errors, as long as the majority of strands have the correct sequence at each position (expected because the positions of errors are generally not correlated between strands), there is a high likelihood that a given error will be replaced with the correct sequence.

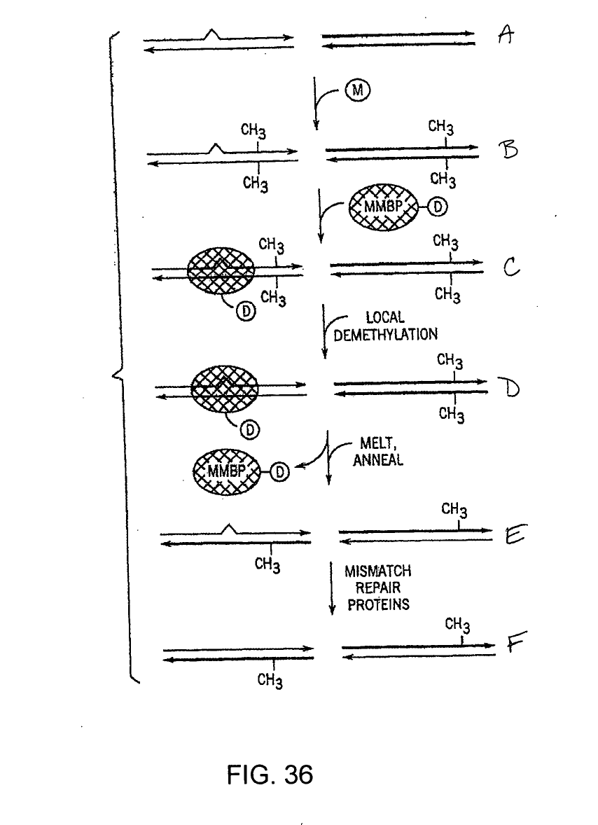

FIG. 36 illustrates an exemplary method for carrying out strand-specific error correction. In replicating organisms, enzyme-mediated DNA methylation is often used to identify the template (parent) DNA strand. The newly synthesized (daughter) strand is at first unmethylated. When a mismatch is detected, the hemimethylated state of the duplex DNA is used to direct the mismatch repair system to make a correction to the daughter strand only. However, in the de novo synthesis of a pair of complementary DNA strands, both strands are unmethylated, and the repair system has no intrinsic basis for choosing which strand to correct. In this aspect of the invention, methylation and site-specific demethylation are employed to produce DNA strands that are selectively hemi-methylated. A methylase, such as the Dam methylase of E. coli, is used to uniformly methylate all potential target sites on each strand. The DNA strands are then dissociated, and allowed to re-anneal with new partner strands. A new protein is applied, a fusion of a mismatch binding protein (MMBP) with a demethylase. This fusion protein binds only to the mismatch, and the proximity of the demethylase removes methyl groups from either strand, but only near the site of the mismatch. A subsequent cycle of dissociation and annealing allows the (demethylated) error-containing strand to associate with a (methylated) strand which is error-free in this region of its sequence. (This should be true for the majority of the strands, since the locations of errors on complementary strands are not correlated.) The hemi-methylated DNA duplex now contains all the information needed to direct the repair of the error, employing the components of a DNA mismatch repair system, such as that of E. coli, which employs MutS, MutL, MutH, and DNA polymerase proteins for this purpose. The process can be repeated multiple times to ensure all errors are corrected.

FIG. 36A shows two DNA duplexes that are identical except for a single base error in the top left strand, giving rise to a mismatch. The strands of the right hand duplex are shown with thicker lines. Methylase (M) may then be used to uniformly methylates all possible sites on each DNA strand (FIG. 36B). The methylase is then removed, and a protein fusion is applied, containing both a mismatch binding protein (MMBP) and a demethylase (D) (FIG. 36C). The MMBP portion of the fusion protein binds to the site of the mismatch thus localizing the fusion protein to the site of the mismatch. The demethylase portion of the fusion protein may then act to specifically remove methyl groups from both strands in the vicinity of the mismatch (FIG. 36D). The MMBP-D protein fusion may then be removed, and the DNA duplexes may be allowed to dissociated and re-associate with new partner strands (FIG. 36E). The error-containing strand will most likely re-associate with a complementary strand which a) does not contain a complementary error at that site; and b) is methylated near the site of the mismatch. This new duplex now mimics the natural substrate for DNA mismatch repair systems. The components of a mismatch repair system (such as E. coli MutS, MutL, MutH, and DNA polymerase) may then be used to remove bases in the error-containing strand (including the error), and uses the opposing (error-free) strand as a template for synthesizing the replacement, leaving a corrected strand (FIG. 36F).

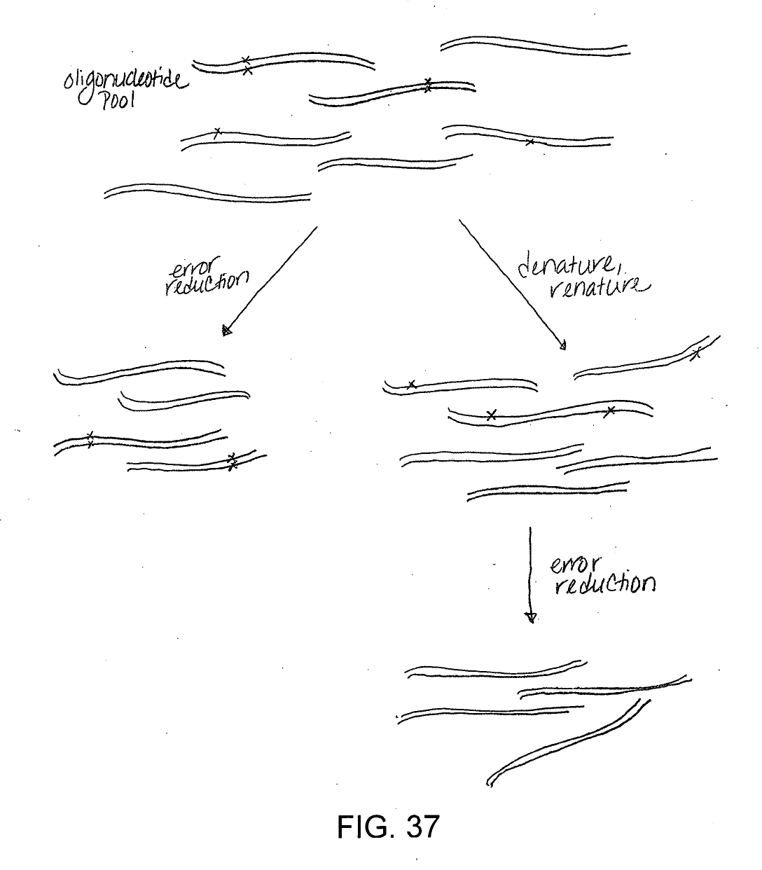

In one embodiment, the number of errors detected and corrected may be increased by melting and reannealing a pool of DNA duplexes prior to error reduction. For example, if the DNA duplexes in question have been amplified by a technique such as the polymerase chain reaction (PCR) the synthesis of new (perfectly) complementary strands would mean that these errors are not immediately detectable as DNA mismatches. However, melting these duplexes and allowing the strands to re-associate with new (and random) complementary partners would generate duplexes in which most errors would be apparent as mismatches (FIG. 37). Since each cycle of error control may also remove some of the error-free sequences (while still proportionately enriching the pool for error-free sequences), alternating cycles of error control and DNA amplification can be employed to maintain a large pool of molecules.

An oligonucleotide sequence bound to a mismatch binding protein can be separated from an unbound oligonucleotide sequences using a variety of methods known in the art including, but not limited to, gel electrophoresis, affinity columns, immunological methods and the like.

Gel electrophoresis is another method by which DNA-protein complexes may be separated from uncomplexed DNA based on migration in a gel medium under the influence of an electric field. DNA-protein complexes exhibit a slower migration rate than uncomplexed DNA and thus can be separated from uncomplexed DNA. Uncomplexed DNA can be removed from the gel using a variety of methods known in the art (Ausubel et al., eds., 1992, current protocols in Molecular Biology, John Wiley & Sons, New York, incorporated by reference herein in its entirety for all purposes).

The invention also provides for selective enrichment of error-free oligonucleotide sequences within a sample by affinity fractionation of oligonucleotide sequences containing errors. Oligonucleotide sequences bound to a mismatch binding protein may be separated from unbound oligonucleotides using affinity fractionation employing a solid support to which mismatch binding protein is coupled. Oligonucleotide sequence-mismatch binding protein complexes are selectively retained by a matrix to which any moiety is coupled which can bind the complex, e.g., a binding protein specific- or complex specific-antibody. This process can be repeated to further enrich oligonucleotide sequences in the eluate have little or no errors.

In addition to antibody supports in which the antibody binds directly to the mismatch binding protein or the oligonucleotide sequence-mismatch binding protein complex, other affinity supports may be used. For example, one can take advantage of the ability of a metal, e.g., nickel, column to bind to histidine residues in a polypeptide using immobilized metal affinity chromatography. A histidine tail, e.g., six histidine residues, may be covalently linked to the amino terminus of the mismatch binding protein, as described by Hochuli et al. ((1988) Biotechnology 6:1321, hereby incorporated by reference in its entirety for all purposes). When the oligonucleotide sequence-mismatch binding protein complex is applied to a nickel column, the histidine portion of the binding protein will be bound by the column.

Another example of an affinity support is an antibody-bound support in which the antibody recognizes and binds to a flag sequence, i.e., any amino acid sequence (e.g., 10 residues) which the antibody specifically binds to. The flag sequence may be engineered onto the amino terminus of the mismatch binding protein. When the oligonucleotide sequence-binding protein complex is applied to the antibody column, the antibody will bind to the flag sequence in the binding protein and thus retain the complex. One embodiment of this technique, known as The Flag Biosystem, is commercially available from International Biotechnologies, Inc. (New Haven, Conn.). Larger flag sequences may be also used, e.g., the maltose binding protein (Ausubel et al., eds., 1992, current protocols in Molecular Biology, John Wiley & Sons, New York, incorporated by reference herein in its entirety for all purposes).

The solid support useful in the invention may be any one of a wide variety of supports, and may include, but is not limited to: synthetic polymer supports, e.g., polystyrene, polypropylene, substituted polystyrene (e.g., aminated or carboxylated polystyrene), polyacrylamides, polyamides, polyvinylchloride, and the like, glass beads, polymeric beads, sepharose, agarose, cellulose, or any material useful in affinity chromatography. The supports may be provided with reactive groups, e.g. carboxyl groups, amino groups, etc., to permit direct linking of the protein to the support. The mismatch binding protein can either be directly crosslinked to the support, or proteins (e.g., antibodies) capable of binding the mismatched binding protein or the nucleic acid/binding protein complex can be coupled to the support.

For example, if the support includes sepharose beads and the mismatch binding protein is coupled to the beads, the binding protein coupled-beads are packed into a column, equilibrated, and the column is subjected to the nucleic acid sample. Under appropriate binding conditions, the protein that is coupled to the beads in the column retains the nucleic acid fragments or the protein/nucleic acid complex which it recognizes.

The protein may be linked to the support by a variety of techniques including adsorption, covalent coupling, e.g., by activation of the support, or by the use of a suitable coupling agent or the use of reactive groups on the support. Such procedures are generally known in the art and no further details are deemed necessary for a complete understanding of the present invention. Representative examples of suitable coupling agents are dialdehydes, e.g., glutaraldehyde, succinaldehyde, or malonaldehyde, unsaturated aldehyde, e.g., acrolein, methacrolein, or crotonaldehyde, carbodiimides, diisocyanates, dimethyladipimate, and cyanuric chloride. The selection of a suitable coupling agent should be apparent to those of skill in the art from the teachings herein.

Another form of affinity purification of oligonucleotide sequence-mismatch binding protein complexes include the use of nitrocellulose filters that bind protein but not free nucleic acid of which are described in Ausubel (1992, supra, incorporated by reference herein in its entirety for all purposes).

Another suitable method of detecting synthetic oligonucleotides having errors is via immunological methods using an antibody such as monoclonal or polyclonal antibody against a mismatch binding protein. An anti-mismatch binding protein antibody can be used to separate mismatch binding protein-oligonucleotide sequence complexes from uncomplexed oligonucleotide sequences by standard techniques, such as affinity chromatography (supra) or immunoprecipitation.

For immunoprecipitation, a mismatch binding protein is precipitated by means of an immune complex which includes the antigen (i.e., mismatch binding protein), primary antibody and Protein A-, G-, or L-substrate conjugate or a secondary antibody-substrate conjugate. The substrate includes, but is not limited to, agarose, beads (e.g., magnetic, glass, polymeric), cells (e.g., S. aureus) and the like. The choice of agarose conjugate depends on the species origin and isotype of the primary antibody. Reagents and protocols for immunoprecipitation are commercially available (e.g., Sigma-Aldrich Co.)

As used herein, the term “antibody” refers to immunoglobulin molecules and immunologically active portions of immunoglobulin molecules, i.e., molecules that contain an antigen binding site which specifically binds (immunoreacts with) an antigen, such as a mismatch binding protein. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind a mismatch binding protein. As used herein, the term “monoclonal antibody” refers to a population of antibody molecules that contain only one species of an antigen binding site capable of immunoreacting with a particular epitope of a mismatch binding protein.

Polyclonal antibodies can be prepared by immunizing a suitable subject with a mismatch binding protein immunogen. The anti-mismatch binding protein antibody titer in the immunized subject can be monitored over time by standard techniques, such as with an enzyme linked immunosorbent assay (ELISA) using immobilized mismatch binding protein. If desired, the antibody molecules directed against a mismatch binding protein can be isolated from the mammal (e.g., from the blood) and further purified by well known techniques, such as protein A chromatography to obtain the IgG fraction.

At an appropriate time after immunization, e.g., when the anti-mismatch binding protein antibody titers are highest, antibody-producing cells can be obtained from the subject and used to prepare monoclonal antibodies by standard techniques, such as the hybridoma technique originally described by Kohler and Milstein ((1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. U.S.A. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies. A New Dimension In Biological Analyses, Plenum Publishing Corp., New York, N.Y. (1980); Lerner (1981) Yale J. Biol. Med. 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a mismatch binding protein immunogen as described above, and the culture supernatants of the resulting hybridoma cells are screened to identify a hybridoma producing a monoclonal antibody that binds the mismatch binding protein. Each reference set forth above is incorporated by reference herein in their entirety for all purposes.

In certain embodiments, it may be desirable to evaluate successful assembly of a subassembly and/or synthetic polynucleotide construct by DNA sequencing, hybridization-based diagnostic methods, molecular biology techniques, such as restriction digest, selection marker assays, functional selection in vivo, or other suitable methods. For example, functional selection may be carried out by introducing a polynucleotide construct into a cell and assaying for expression of one or polynucleotides on the construct. Successful assemblies may be determined by assaying for a detectable marker, a selectable marker, a polypeptide of a given size (e.g., by size exclusion chromatography, gel electrophoresis, etc.), or by assaying for an enzymatic function of one or more polypeptides encoded by the polynucleotide construct. DNA manipulations and enzyme treatments are carried out in accordance with established protocols in the art and manufacturers' recommended procedures. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985).

In certain embodiments, the polynucleotide constructs may be introduced into an expression vector and transfected into a host cell. The host cell may be any prokaryotic or eukaryotic cell. For example, a polypeptide of the invention may be expressed in bacterial cells, such as E. coli, insect cells (baculovirus), yeast, plant, or mammalian cells. The host cell may be supplemented with tRNA molecules not typically found in the host so as to optimize expression of the polypeptide. Ligating the polynucleotide construct into an expression vector, and transforming or transfecting into hosts, either eukaryotic (yeast, avian, insect or mammalian) or prokaryotic (bacterial cells), are standard procedures. Examples of expression vectors suitable for expression in prokaryotic cells such as E. coli include, for example, plasmids of the types: pBR322-derived plasmids, pEMBL-derived plasmids, pEX-derived plasmids, pBTac-derived plasmids and pUC-derived plasmids; expression vectors suitable for expression in yeast include, for example, YEP24, YIP5, YEP51, YEP52, pYES2, and YRP17; and expression vectors suitable for expression in mammalian cells include, for example, pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors.

Embodiments of the present invention are further directed to an article of manufacture (e.g., a kit, an automated system) that provides at least one reservoir containing a plurality of different polynucleotides having different primer sequences (i.e., construct reservoirs), and reservoirs containing primers (i.e., primer reservoirs). In certain aspects, the articles of manufacture contain at least one reservoir containing a plurality of different polynucleotides and the primers are provided by the user. Various combinations of primers can be chosen to amplify specific polynucleotide sequences. A variety of different polynucleotides may be retrieved from a single reservoir as each polynucleotide comprises a unique set of amplification primers. In certain aspects, the plurality of different polynucleotides comprise nested primer sequences. A polynucleotide reservoir may include 10², 10³, 10⁴, 10⁵, 10⁶, 10⁷, 10⁸, 10⁹, 10¹⁰ or more different polynucleotide sequences.

The portion of the articles of manufacture that provides the reservoirs may be manufactured from a variety of materials known in the art including, but not limited to, a variety of plastics, polymers, glasses and combinations thereof, and may be in the form of, for example, microtitre plates (e.g., 384 well plates), microchips, tubes (e.g., PCR tubes, microfuge tubes, test tubes, tissue culture plates, etc.) and the like.

In certain aspects, the plurality of different polynucleotides and/or the primers are covalently attached to one or more reservoirs. Accordingly, the articles of manufacture provided herein are reusable in that one or more polynucleotide sequences and/or primer sets may be repeatedly amplified simply by adding additional primer pairs specific to the polynucleotide sequence that one wishes to amplified together with polymerase and nucleotides. Suitable methods of amplification are described further herein. The articles of manufacture described herein are useful for amplifying polynucleotides corresponding to genes, gene sets, genomes, vectors and the like.

Any of the methods of making synthetic polynucleotides described herein may be performed using an automated amplification systems. In certain aspects, at least portions of the articles of manufacture described herein include automated components. As such, the articles of manufacture may include data storage (e.g., that lists the polynucleotides and/or primer pairs provided), an interface permitting a user to specify a polynucleotide or group of polynucleotides to be amplified, and an automated means responsive to specifications input at the interface. Instructions may be accessed from data storage for extracting aliquots of polynucleotides from one or more construct reservoirs and from one or more primer reservoirs to prepare one or more amplified polynucleotide sequences.

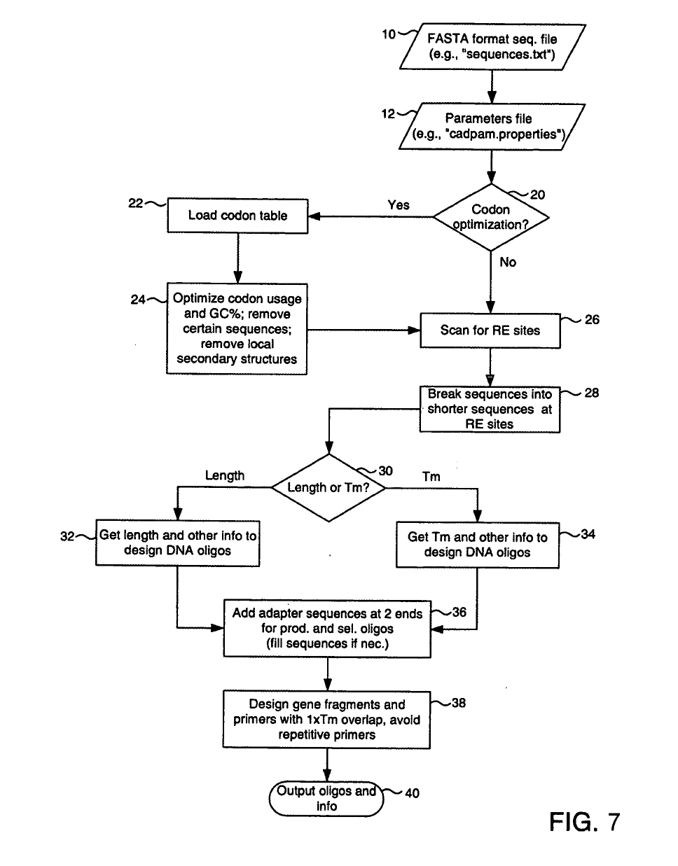

Embodiments of the invention include the use of computer software to automate design of gene and oligonucleotide sequences. Such software may be used in conjunction with individuals performing polynucleotide synthesis by hand or in a semi-automated fashion or combined with an automated synthesis system. In at least some embodiments, the gene/oligonucleotide design software is implemented in a program written in the JAVA programming language. The program may be compiled into an executable that may then be run from a command prompt in the WINDOWS XP operating system. Operation of this software (named “CAD-PAM,” for Computer Aided Design-Polymerase Assembly Multiplexing) is described in this section and in FIGS. 7-27. However, CAD-PAM is merely one embodiment of various aspects of the invention. Unless specifically set forth in the claims, the invention is not limited to an implementation including all features of CAD-PAM or to implementations using the same algorithms, organizational structure or other specific features of CAD-PAM. The invention is similarly not limited to implementation using a specific programming language, operating system environment or hardware platform.

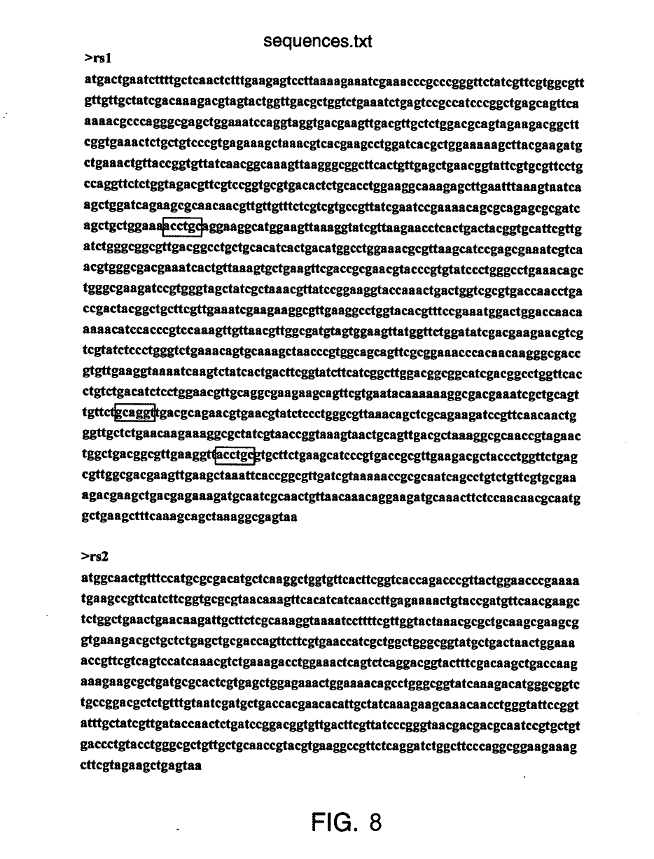

FIG. 7 is a flow chart showing operation of the CAD-PAM program. The program receives two inputs. The first (block 10) is a file (“sequences.txt”) containing one or more nucleotide sequences (e.g., gene sequences), in FASTA format, for which selection and construction oligonucleotides are to be designed. FIG. 8 shows an example of an input sequences file. The rectangles shown in the sequence rs-1 are included to indicate portions of the sequence which will be discussed below. Although the file shown in FIG. 8 contains two sequences (rs-1 and rs-2), only a single sequence (or more than two sequences) could be input. The second input to CAD-PAM is a file (“cadpam.properties,” block 12) containing parameters controlling design of oligonucleotides.

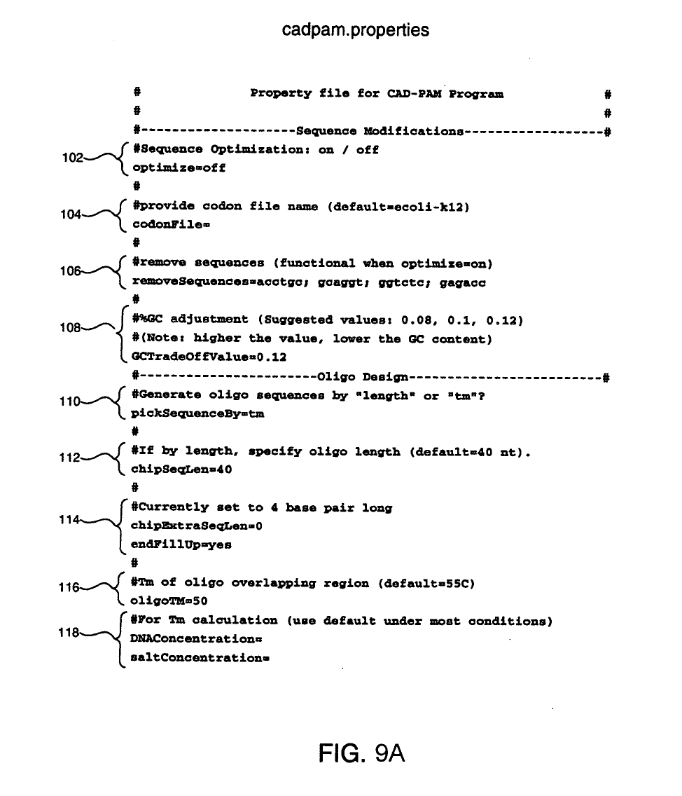

FIGS. 9A and 9B show an example of a cadpam.properties input parameter file. Beginning in FIG. 9A, and as shown at bracket 102, a first parameter (“optimize=”) specifies whether the input sequence(s) (in the sequences.txt file, FIG. 8) are to be modified based on codons most frequently used by an organism which will express one or more nucleotide sequences of the input sequence(s). In the example of FIG. 9A, this parameter is set to “optimize=off”. Accordingly, the input sequences will not be modified. If the parameter were set to “on” (“optimize=on”), and as described in more detail below, the input sequences would be modified based on codons used by the expressing organism. Information about the expressing organism is supplied by the user in a separate file. If no file is specified, information regarding a default organism (e.g., E. coli K12) is used. The name of a file for a non-default organism is provided as the next parameter (“codonFile=”, shown at bracket 104). The content of such a file is further discussed below.

The next input parameter in FIG. 9A is “removeSequences” (bracket 106). This parameter specifies nucleotide sequences which are to be removed from input sequences; further details regarding operation of this parameter are provided below. Following the removeSequences parameter is the parameter “GCTradeOffValue” (bracket 108). This parameter provides additional control over the organism-specific optimization of a nucleotide sequence by adjusting the GC content of the optimized sequence. Further details of the operation of this parameter are also provided below.

The next set of input parameters in FIG. 9A (under “Oligo Design”) control the design of construction and selection oligonucleotides which will be used to create the desired gene sequences (i.e., the sequences specified in sequences.txt, including any organism-specific modification). The parameter shown at bracket 110 (“pickSequenceBy”) specifies whether oligonucleotides will be designed based only on the Tₘ of the overlapping ends of the designed oligonucleotides (pickSequenceBy=Tₘ) or based on length of the oligonucleotide (pickSequenceBy=length). If pickSequenceBy=length, a length (in number of nucleotides) is specified as the “chipSeqLen” parameter (bracket 112). If pickSequenceBy=length and a length is not specified, a default value (e.g., 40 nucleotides) is used.

Following the chipSeqLen parameter are the “chipExtraSeqLen” and “endFillUp” parameters at bracket 114. The chipExtraSeqLen parameter specifies the length of a sticky end of a construction oligonucleotide which may remain as a result of restriction enzyme (RE) cleavage. The endFillUp parameter specifies whether extra sequences will be added to make the oligonucleotides of equal length. The lengths of construction oligonucleotides or selection oligonucleotides can be constant or variable. Extra sequences can be added to either or both ends of the oligonucleotides. Added sequences are chosen from the native nucleic acid sequence in the gene adjacent to the construction oligonucleotide.

Shown at bracket 116 is the parameter “oligo™”. This parameter allows specification of a Tₘ for overlapping portions of designed oligonucleotides. Shown at bracket 118 are the parameters “DNAConcentration” and “saltConcentration”. These parameters allow input of specific values for solution concentration of DNA strands and salt during sequence specific hybridization of oligonucleotides. As discussed in more detail below, these values are used when calculating the Tₘ of the overlapping oligonucleotide segments.

The parameter input file continues in FIG. 9B. In the first section of FIG. 9B (under “Oligo Chip-Synthesis”) are parameters “sense5endAddOn” and “sense3endAddOn” (bracket 120). These parameters, which are discussed more fully below, specify sequences to be added to the 5′ and 3′ ends of each construction oligonucleotide. These sequences could be, e.g., restriction enzyme recognition sites. The parameters “selection5endAddOn” and “selection3EndAddOn” (bracket 122) are also discussed below, and specify sequences to be added to the 5′ and 3′ ends of selection oligonucleotides. At bracket 124 is the parameter “selectionFillUpLen,” which specifies a limit on the number of adenine bases which may be added to a selection oligonucleotide in order to reach a desired oligonucleotide length. The parameter “selectionChip™” (bracket 126) is a Tₘ for the portions of selection oligonucleotides overlapping portions of construction oligonucleotides.

The final section of FIG. 9B contains the parameters “reSite” and “poolSize” (brackets 128 and 130, respectively). The reSite parameter identifies restriction enzyme (RE) sites at which a sequence may be broken into smaller sequences. These sites may (but need not be) be the same as the sequences previously identified by the “removeSequences” parameter. In at least some embodiments, the multiple RE sites are provided in the format <RE site 1 in 5′-3′ direction>; <RE site 1 in 3′-5′ direction>; <RE site 2 in 5′-3′ direction>; <RE site 2 in 3′-5′ direction>; etc. The poolSize parameter sets a limit on the number of fragments into which an input sequence may be cut to create construction oligonucleotides. The operation of the poolSize parameter is also discussed below.

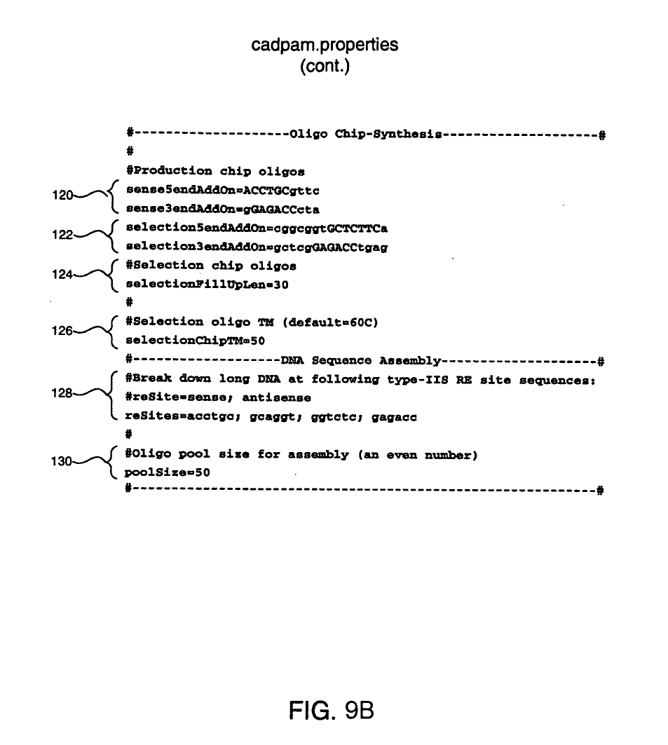

After receiving the sequences.txt and cadpam.properties inputs, the program proceeds to block 20. At decision block 20, the program determines whether optimization based on expressing organism codon usage is desired (i.e., whether the “optimize” parameter from FIG. 9A is “on” or “off”). If optimization is not desired, the program proceeds on the “No” branch from block 20 to block 26. Block 26 is discussed below. If optimization is desired, the program proceeds on the “Yes” branch from block 20 to block 22. At block 22, a codon table for either a user-specified or a default organism is loaded. FIGS. 10A and 10B show a codon usage table for default organism E. coli K12. The table of FIGS. 10A and 10B, which is in a standard GCC-normal format, is similar to codon usage tables available for numerous organisms. One source of such tables can be found online at <http://www.kazusa.orjp/codon/>. Column 140 lists abbreviations for the twenty amino acids, and column 142 lists codons used to code each of those twenty amino acids. Column 148 lists a usage percentage of each codon for a specific organism. For example, the first four rows in FIG. 10A correspond to glycine (“Gly”). Of the four nucleotide triplets that encode glycine, GGG is used by E. coli K12 15% (i.e., 0.15) of the time to encode glycine. GGA, GGT and GGC are used 11%, 34% and 40%, respectively. Columns 144 and 146 are not used by at least some embodiments of the invention, but have been left in place because they are part of the standard GCC-normal format. A codon usage table for another organism would be in the same format, but have different values in columns 144-148 corresponding to that other organism.

As part of loading the codon usage table at block 22, the program adjusts codon usage percentages in the table based on the GC content of each codon. Although it may be desirable to replace a particular codon in a sequence with another codon that is used more frequently by an expressing organism for the same amino acid, it may also desirable to minimize the GC content of the sequence in order to improve overall expression by that organism. Because these are sometimes competing goals (i.e., the codon with the highest usage percentage may also be the codon with the highest GC content), a trade-off between these two criteria can be specified with the GCTradeOffValue parameter (FIG. 9A). For each codon in the usage table having two or three G or C bases, GCTradeOffValue is subtracted from the usage percentage of that codon. If GCTradeOffValue=0.12, for example, the GGG and GGA codons of FIG. 10A have their usage percentages reduced to −0.21 (0.15-0.12-0.12-0.12) and 0.0 (0.11-0.12, with negative values rounded to 0), respectively. For each codon in the usage table having zero or one G or C bases, GCTradeOffValue is added to that codon's usage percentage. In the present example of GCTradeOffValue=0.12, two of the codons for threonine (ACA and ACT) have their usage percentages increased (to 0.25 and 0.29, respectively).

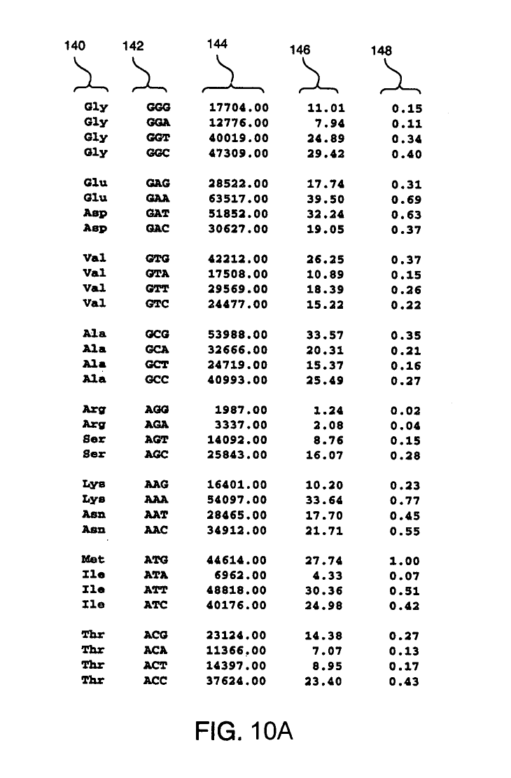

After loading a codon table at block 22, the program proceeds to block 24. At block 24, the program then optimizes the input sequences (from the sequences.txt file) based on the loaded codon table and on other parameters specified in the cadpam.properties file. Shown in FIG. 11 is a flow chart describing the optimization procedure. Beginning in block 24-1, the program examines the first three bases in the input sequence. If multiple sequences are included in the sequences.txt file, the optimization procedure of FIG. 11 is performed serially on each sequence (i.e., the procedure is carried through on the first sequence, and then on the next sequence, etc.). In block 24-3, the program compares the bases being examined with the codon usage table loaded at block 22 (FIG. 11), and identifies the codon for the same amino acid having the highest usage percentage (after adjustment if GCTradeOffValue is not equal to zero and optimize=on). The program then substitutes the highest-usage codon for the original codon at block 24-5. In some cases (e.g., the original codon is the most used codon and has low GC content), the program will effectively be replacing a codon with the same codon.

From block 24-5, the program proceeds to block 24-7 and determines if there are more codons in the sequence. If so, the program proceeds on the “yes” branch to block 24-9 and examines the next three bases in the sequence. From block 24-9 the program then returns to block 24-3 and repeats blocks 24-3 through 24-7 for those next three bases. If at block 24-7 the program has reached the end of the sequence, the program proceeds on the “no” branch to block 24-11.

At block 24-11, the program looks for secondary structure in the sequence and replaces that secondary structure with alternate codons. In particular, the program searches along the entire sequence for combinations of bases that may form loops, hairpins, etc. In at least some embodiments, the program performs this search by looking for self-complementary sequences within a given region. Upon finding a secondary structure, the program then replaces the codon(s) of the secondary structures with alternate codons encoding the same amino acids. In some embodiments, the replacement codons are selected at block 24-11 by selecting an alternate codon from the usage table having the highest usage percentage.

In some embodiments, the steps of block 24-11 are repeated until the entire sequence can be traversed without identifying a secondary structure, or until some other stop condition is reached (e.g., passing through the sequence a certain number of times). For example, replacing one or more codons to eliminate a secondary structure in one region could inadvertently introduce a secondary structure in another region of the sequence. If this occurs, the inadvertently created secondary structure is corrected on the next pass through the sequence. For simplicity, alternate embodiments in which block 24-11 is repeated are shown with a broken line arrow.

After completing block 24-11 (or completing all repetitions of block 24-11), the program proceeds to block 24-13. At block 24-19, the program searches the sequence for base combinations identified in the removeSequences parameter of the cadpam.properties file (FIG. 9A). Upon finding such a base combination, the program replaces those bases with codons encoding the same amino acids. In some embodiments, the replacement codons are selected at block 24-13 by selecting an alternate codon from the usage table having the highest usage percentage. In some embodiments, and for reasons similar to those described for block 24-11, block 24-13 is repeated until the entire sequence is traversed without finding a removeSequences base combination or until some other stop condition is reached.

After block 24-13, the program returns to the main program flow of FIG. 7 and proceeds block 26. At block 26 the program scans the optimized input sequence (or the original input sequence if block 26 is reached directly from block 20) for the RE sites identified by the reSite parameter (FIG. 9B). At block 28, and if any of those RE sites are found, the program divides the sequence at those found sites. The program divides the input sequences at the RE sites so that subsequently designed construction oligonucleotides will not have such sites in unwanted locations (e.g., in the middle of a construction oligonucleotide sequence).

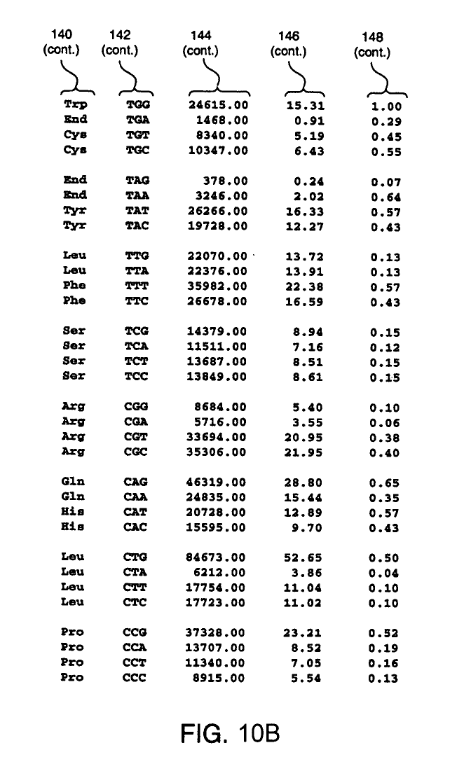

The division of a sequence at block 28 is seen by comparing FIG. 12 with FIG. 8. FIG. 12 shows input sequence rs1 divided into four shorter sequences rs1-f1, rs1-f2, rs1-f3 and rs1-f4. Because sequence rs2 contained none of the specified RE sites, sequence rs2 was not divided. The locations within rs1 of the specified RE sites are shown with boxes in FIG. 8. At each of those sites, the RE site is split in the center. Thus, for example, the division between rs 1-f1 and rs1-f2 occurs in the middle of the RE site acctgc shown in the first box of FIG. 8. Partial boxes around ends of the shorter sequences rs1-f1 through r31-f4 (FIG. 12) represent halves of the boxes of FIG. 12.

The program then proceeds to decision block 30. At block 30, the program determines whether oligonucleotides will be designed based on Tₘ or based on oligonucleotide length. If the input parameter pickSequenceBy (bracket 110, FIG. 9A) equals “tm,” the program proceeds to block 34 and designs construction and selection oligonucleotides based on Tₘ of the overlapping portions of designed construction oligonucleotides.

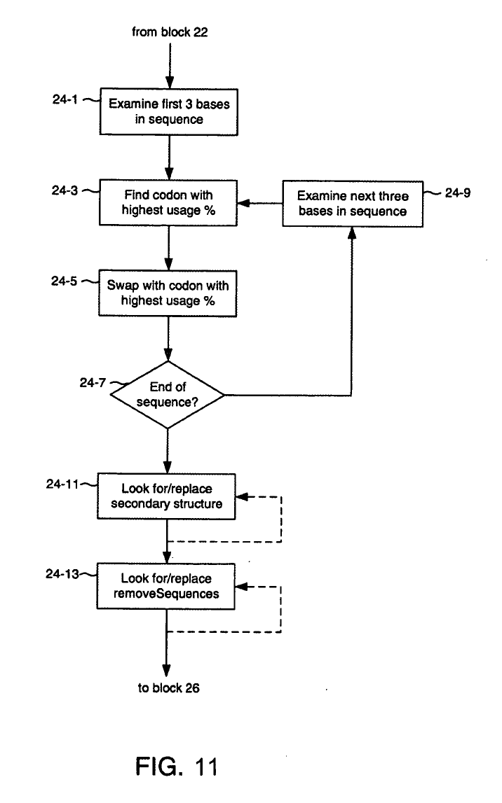

Operation of the program in block 34 is shown in more detail FIGS. 13A through 18. FIGS. 13A and 13B are flowcharts showing steps of an algorithm, according to at least some embodiments, followed in block 34 of FIG. 7. Beginning in block 34-1 (FIG. 13A), the program retrieves the first sequence for which construction and selection oligonucleotides are to be created. Using the inputs of FIG. 8 as an example, and after division of sequence rs1 into shorter sequences as described above, the sequences to be analyzed in the algorithm of FIGS. 13A-B are rs1-f1, rs1-f2, rs1-f3, rs1-f1 and rs-2. Accordingly, the program selects the first of these (rs1-f1) for analysis at block 34-1.

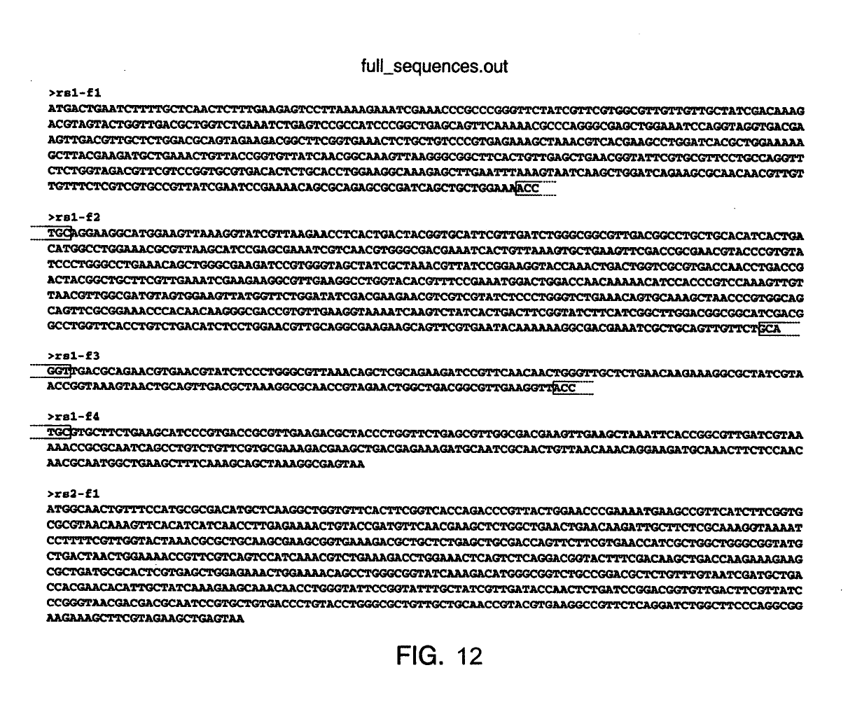

The program then proceeds to block 34-3 and places a start point at the 3′ end of the sequence selected in block 34-1. This is shown diagrammatically in FIG. 14, where the start point is shown as a triangle placed at the 3′ end of sequence rs1-f1. The program then proceeds to block 34-5. At block 34-5, the program identifies a search window extending a predetermined number (W) of bases from the start point toward the 5′ end of rs1-f1. In at least some embodiments, the search window length is set such that W equals Tₘ (FIG. 9A, bracket 116) rounded off to the nearest integer. In the present example, W=50 bases. The program then proceeds to block 34-7, where the program determines if the search window would overrun the 5′ end of the current sequence. Stated differently, the program determines if W bases from the start point extends beyond the 5′ end of the current sequence. If so, the program proceeds on the “yes” branch to block 34-21, which is discussed below. If not, the program proceeds on the “no” branch to block 34-9.

At block 34-9, the program then identifies an overlap region in the search window. As will be explained below, the sequence being analyzed by the program is further divided into a collection of overlapping fragments. In order to identify an overlap region within a search window, the program searches for a region having a melting point Tₘ closest to the desired value for Tₘ specified in the input parameters (bracket 116, FIG. 9A). FIG. 13B shows in more detail the operation of the program in block 34-9. At block 34-9-1, the program determines if the start point is currently at the 3′ end of the sequence being analyzed. If so, the program proceeds on the “yes” branch to block 34-9-3. At block 34-9-3, the program then moves an offset distance toward the 5′ end within the search window. This is also shown diagrammatically in FIG. 14. The program moves the offset distance in the 5′ direction so that an overlap region will not commence at the 3′ end of the sequence. If this were to occur, the overlap region would consume the entire search window. As will be seen below, this would result in a construction oligonucleotide that is completely overlapped by another construction oligonucleotide.

After moving an offset distance toward the 5′ end, the program proceeds to block 34-9-5. In block 34-9-5, and as also shown in FIG. 14, the program searches for a region within the search window having a melting point closest to the Tₘ value specified in the input parameters. In at least some embodiments, melting point is calculated using the nearest neighbor method, taking into account the values for DNAConcentration and saltConcentration specified by the input parameters (bracket 118, FIG. 9A). The nearest neighbor method of melting point calculation is known in the art, and is described in Breslauer et al. (1986) Proc. Natl. Acad. Sci. U.S.A. 83:3746 (supra). Computer algorithms implementing the nearest neighbor method are known in the art and thus not further described herein.

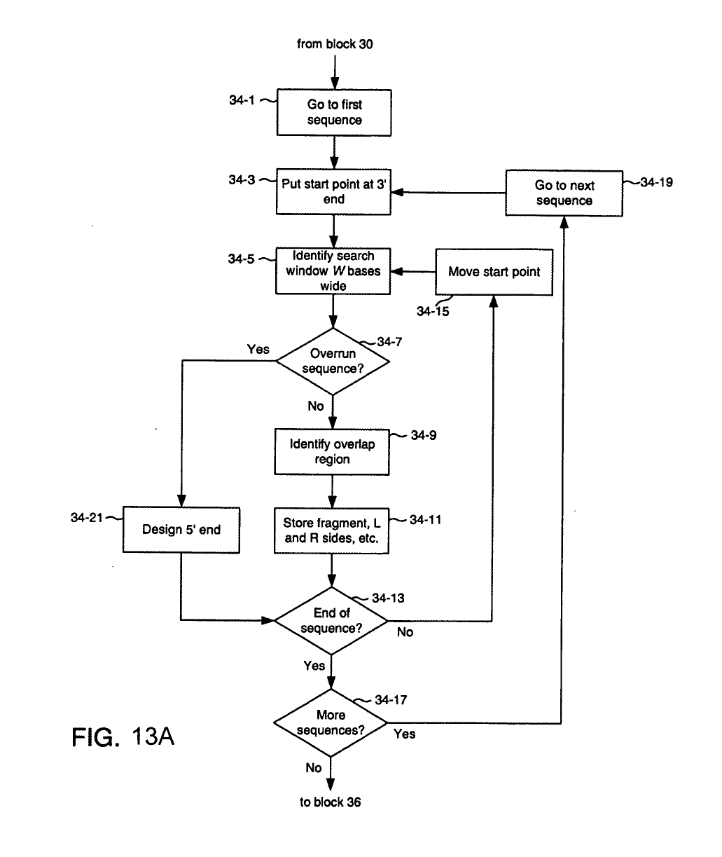

FIG. 15 diagrammatically shows the 3′ end of rs1-f1 after an overlap region (underlined) having a melting point closest to the input Tₘ value is found in block 34-9-5. As seen in FIG. 15, the overlap region defines a first oligonucleotide fragment (rs1-f1-1). The overlap region is the left side of the fragment (rs1-f1-1L), and the portion between the 3′ end of the overlap region and the 3′ end of the fragment is the right side of the fragment (rs1-f1-1R). At block 34-11 (FIG. 13A), the bases in rs1-f1-1, rs1-f1-1L and rs1-f1-1R are stored, and the program proceeds to block 34-13. At block 34-13, the program determines if it has reached the end of the sequence being analyzed. If not, the program proceeds on the “no” branch to block 34-15. At block 34-15, the start point is moved to the 3′ end of the previously-identified overlap region (as shown in FIG. 15), and the program returns to block 34-5.

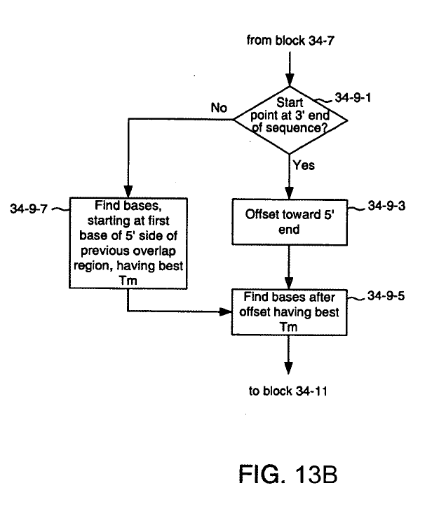

After returning to block 34-5, the program repeats block 34-7 and (assuming a “no” is determined at block 34-7) block 34-9. In this case, however, the start point is no longer at the beginning of the sequence, and the program thus proceeds on the “no” branch from block 34-9-1 (FIG. 13B) to block 34-9-7. At block 34-9-7, the program then determines the next overlap region as shown in FIG. 16. Beginning at the first base on the 5′ side of the previously-found overlap region (rs1-f1-1L), the program moves toward the 5′ end of the search window and determines the bases contiguous to rs1-f1-1L having a melting point closest to the desired Tₘ. Once these bases are found (shown with double underlining in FIG. 16), the program proceeds to block 34-11 (FIG. 13A) and stores the portion of the sequence defined by the latest and the previous latest overlap regions as the next oligonucleotide fragment (rs1-f1-2). The latest overlap region becomes rs1-f1-2L, and the previous overlap region (rs1-f1-1L) is also rs1-f1-2R. The program then proceeds to block 34-13.

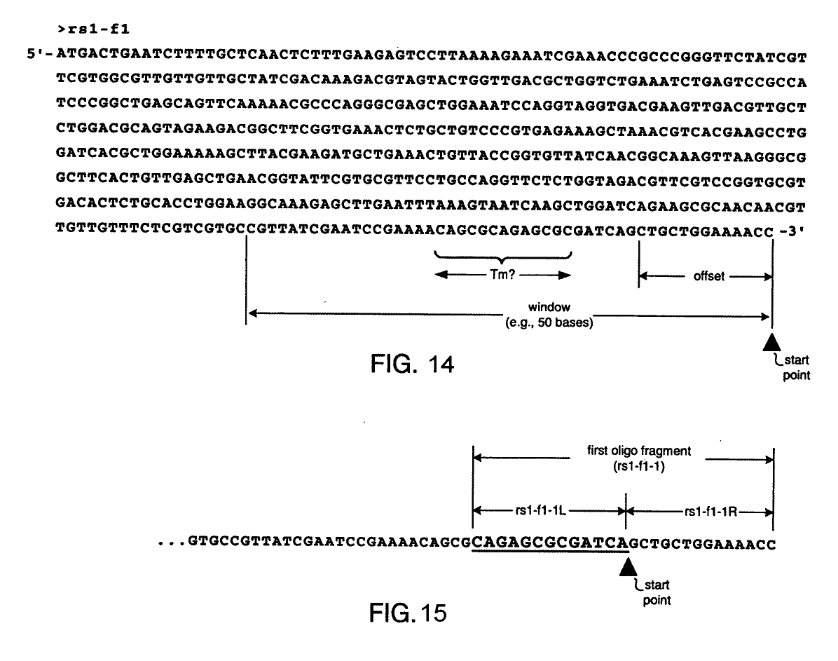

FIG. 17 diagrammatically shows operation of the program when the end of a sequence is reached. This corresponds to the “yes” branch from block 34-7 (FIG. 13A) and block 34-21. As shown in FIG. 17, the program adds bases as needed to achieve a desired Tₘ. A portion of a construction oligonucleotide corresponding to a fragment with these added bases can later be excluded from a gene or sequence being constructed. The final fragment (rs1-f1-n, or in the example, rs1-f1-38) is defined by the previous overlap region, the remaining 5′ end of the fragment being examined, and the added bases. This information is stored, and the program proceeds to block 34-13. At block 34-13, the end of the sequence has been reached, and the program proceeds on the “yes” branch to block 34-17. At block 34-17, the program determines if there are additional sequences to be analyzed. If so, the program proceeds on the yes branch to block 34-19 and goes to the next sequence (e.g., rs1-f2 in FIG. 12). If not, the program proceeds on the “no” branch to block 36 (FIG. 7).

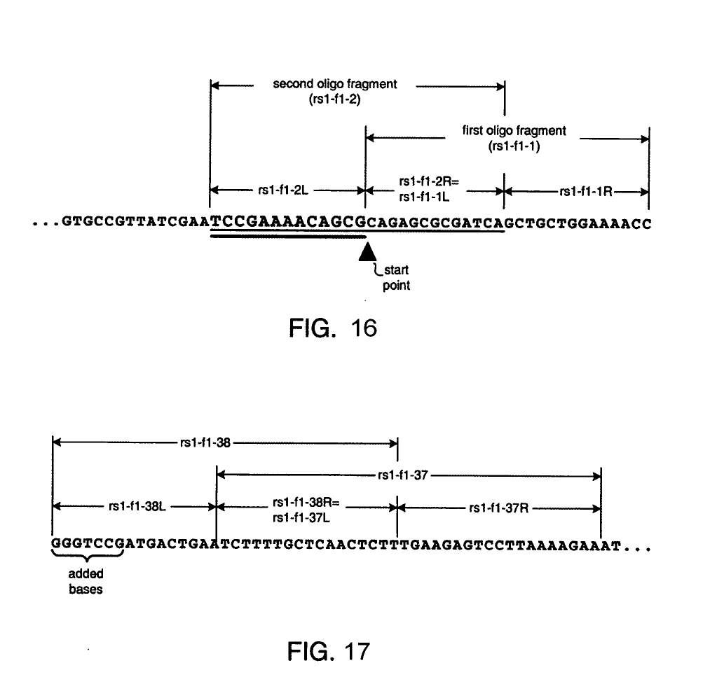

FIG. 18 shows a portion of an output file (in the example, titled “info.out”) containing data generated by the program during the steps shown in FIGS. 13A-13B. Some of the data shown in FIG. 18 is generated by the program in subsequent steps, as described below.

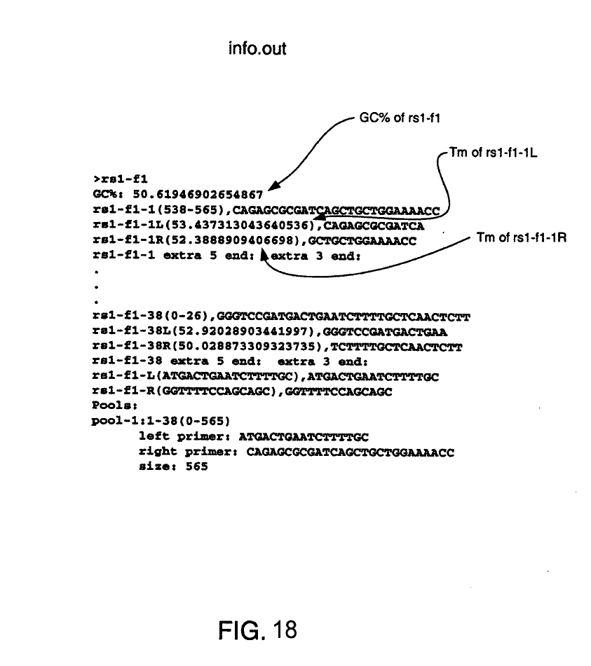

If the input parameter pickSequenceBy (bracket 110, FIG. 9A) were instead set to “length” instead of “tm,” the program would proceed from block 30 (FIG. 7) to block 32. Operation of the program in block 32 is shown in more detail FIGS. 19 through 22. FIG. 19 is a flowchart showing steps of an algorithm, according to at least some embodiments, followed in block 32 of FIG. 7. Beginning in block 32-1, the program retrieves the first sequence or sequence for which construction and selection oligonucleotides are to be designed. Again using the inputs of FIG. 8 as an example, the program initially selects rs1-f1 for analysis at block 32-1.

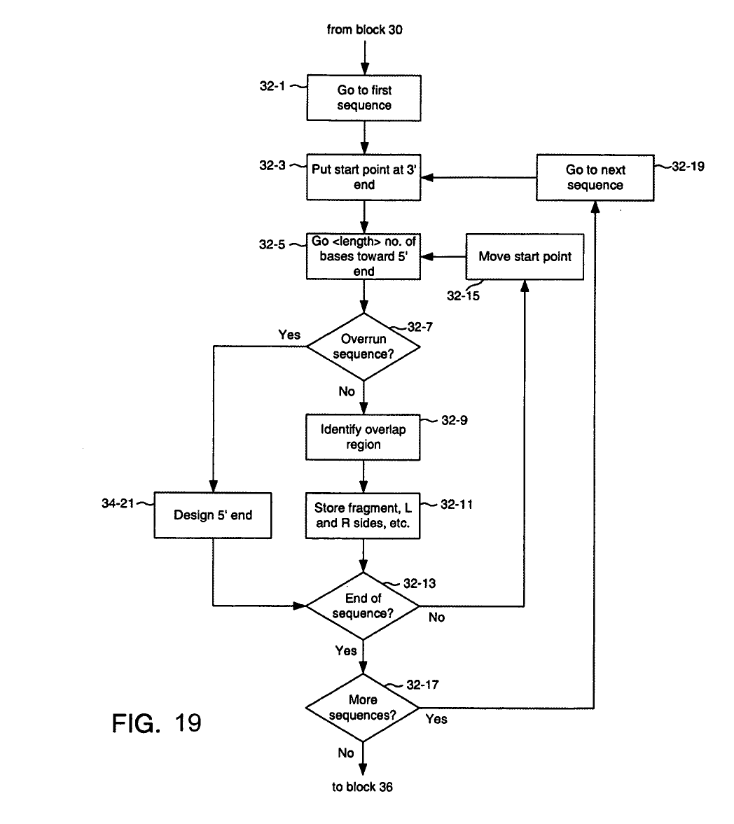

The program then proceeds to block 32-3 and places a start point at the 3′ end of the sequence selected in block 32-1. This is shown diagrammatically in FIG. 20, where the start point is shown as a triangle placed at the 3′ end of sequence rs1-f1. The program then proceeds to block 32-5. At block 32-5, the program attempts to identify a number of bases, extending from the start point toward the 5′ end of the current sequence, corresponding to the input “chipSeqLen” parameter (bracket 112, FIG. 9A). In the example of FIGS. 19-22, it is assumed that chipSeqLen=40 bases. The program proceeds to block 32-7, where the program determines if it has overrun the 5′ end of rs1-f1. Stated differently, the program determines if chipSeqLen bases from the start point extends beyond the 5′ end of rs-f1. If so, the program proceeds on the “yes” branch to block 32-21, which is discussed below. If not, the program proceeds on the “no” branch to block 32-9.

In block 32-9, the length-based fragment identified in step 32-5 becomes rs1-f1-1 (FIG. 20). The program determines the overlap region for rs1-f1-1 by starting at the 5′ end of rs1-f1-1 and identifying the bases at the 5′ end of rs1-f1-1 having a melting temperature closest to a desired value (input parameter “tm” of bracket 110, FIG. 9A). Because the oligonucleotide fragments are now being chosen based on a required length, a larger range of Tₘ values for overlap regions may be required. Once the overlap region is identified, the program proceeds to block 32-11. At block 32-11, the program stores data for the bases in rs1-f1-1, rs1-f1-1L (the overlap region found in block 32-9), and rs1-f1-1R (a portion of rs1-f1-1 at the 3′ end having a Tₘ closest to a desired Tₘ).

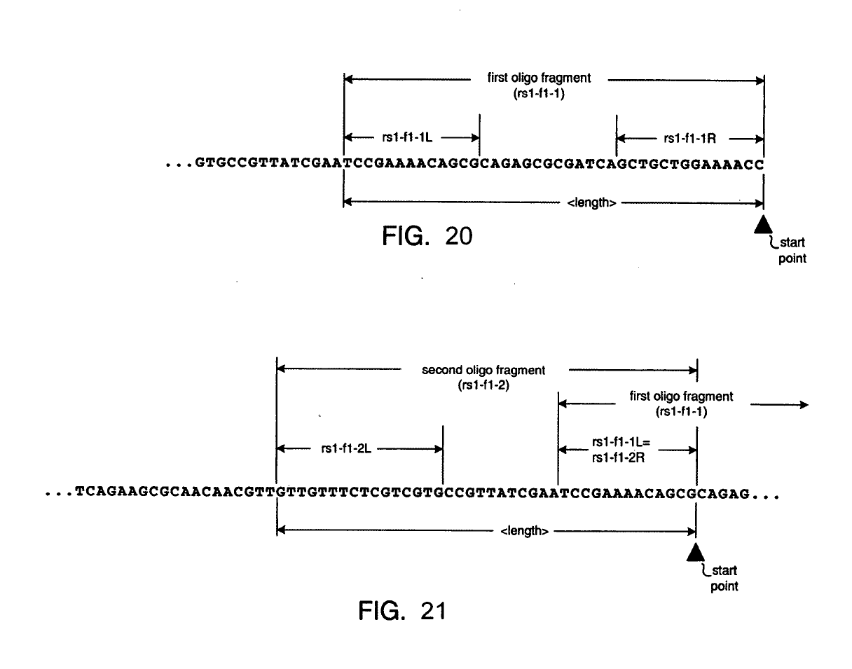

The program then proceeds to block 32-13 and determines if the end of current sequence has been reached. If not, the program proceeds on the “no” branch to block 32-15, and places the start point at the 3′ end of the overlap region just identified. This is shown in FIG. 21. The program then returns to block 32-5 and repeats steps of blocks 32-5, 32-7 and (assuming the end of the current sequence has not been overrun) 32-9 through 32-13. FIG. 21 diagrammatically shows the determination of the second length-based oligonucleotide fragment (rs1-f1-2) and its left and right portions. In the case of second and subsequent length-based fragments, the right side is set to equal the left side of the prior fragment (e.g., rs1-f1-2R is the same as rs1-f1-1L).

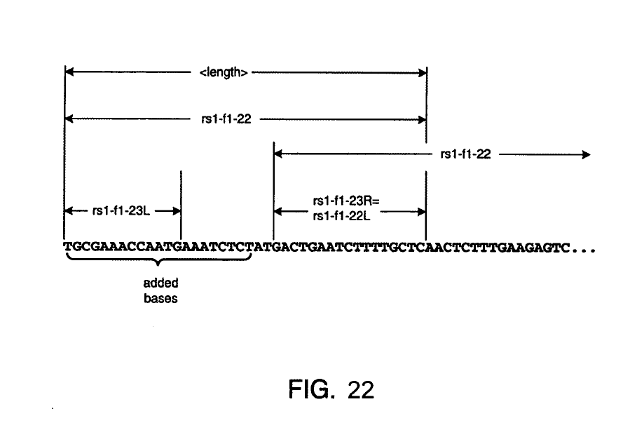

FIG. 22 diagrammatically shows operation of the program when the end of a sequence is reached. This corresponds to the “yes” branch from block 32-7 (FIG. 19) and block 34-21. As shown in FIG. 22, the program adds bases as needed to achieve the specified length and to obtain a left end having a melting point that is as close as possible to the desired Tₘ. The section of a construction oligonucleotide corresponding to these added bases can later be excluded from a gene or sequence being constructed. The final fragment (rs1-f1-n, or in the example, rs1-f1-23), together with its left and right ends, is shown in FIG. 22. This information is stored, and the program proceeds to block 32-13. At block 32-13, the end of the sequence has been reached, and the program proceeds on the “yes” branch to block 32-17. At block 32-17, the program determines if there are additional sequences to be analyzed. If so, the program proceeds on the yes branch to block 32-19 and goes to the next sequence (e.g., rs1-f2 in FIG. 12). If not, the program proceeds on the “no” branch to block 36 (FIG. 7).

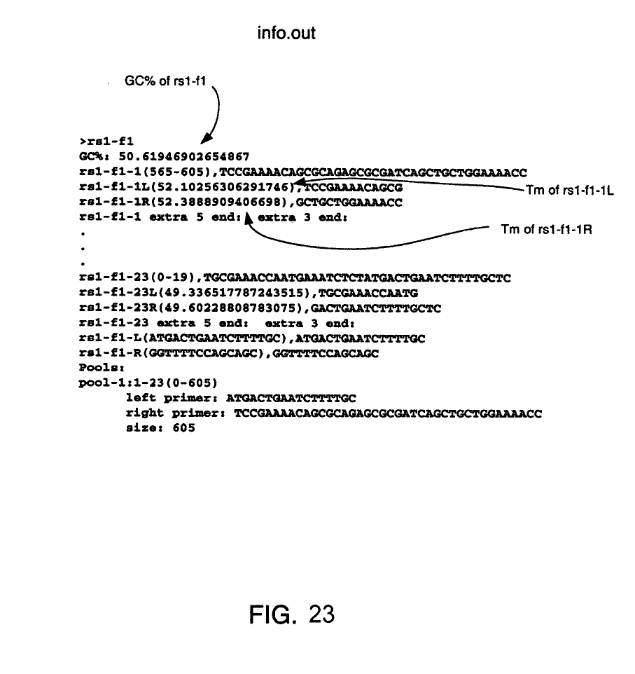

FIG. 23 shows a portion of an output file (in the example, titled “info.out”) containing data generated by the program during the steps shown in FIGS. 19-22. Some of the data shown in FIG. 23 is generated by the program in subsequent steps, as described below.

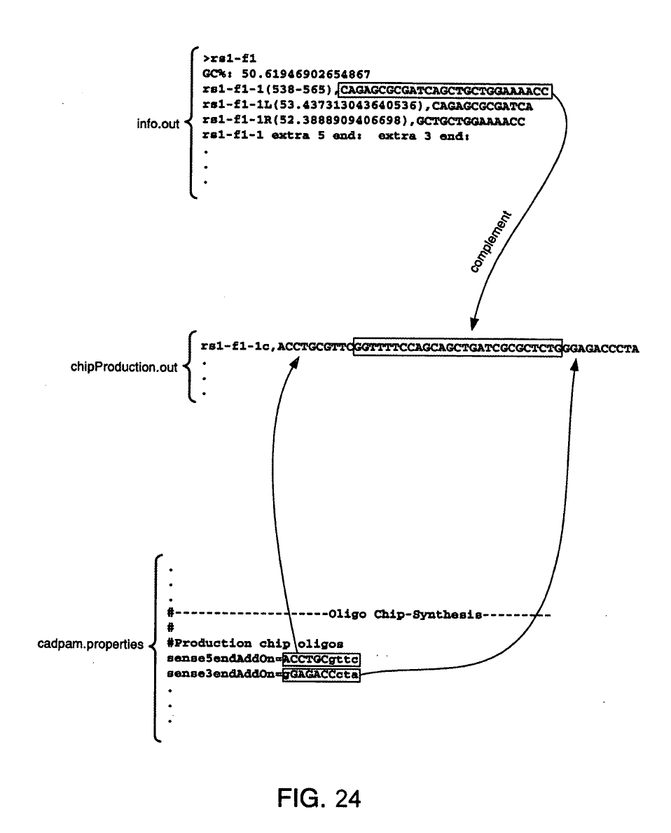

In block 36, construction and selection oligonucleotides are generated based on the fragments (e.g., rs1-f1-1, rs1-f1-2, etc.) determined in block 32 or block 34. FIG. 24 diagrammatically shows how construction oligonucleotides are generated, and shows portions of the info.out file of FIG. 18, the cadpam.properties file of FIG. 9B, and a third file (named “chipProduction.out”) containing the generated construction oligonucleotides. The first construction oligonucleotide (rs1-f1-1c) is generated by taking the complement of rs1-f1-1 (info.out) and appending the sequences identified by the “sense5endAddOn” and “sense3endAddOn” input parameters (from cadpam.properties). The remaining construction oligonucleotides (e.g., rs1-f1-2c) for rs1-f1 (and other sequences being processed) are generated in a similar manner.

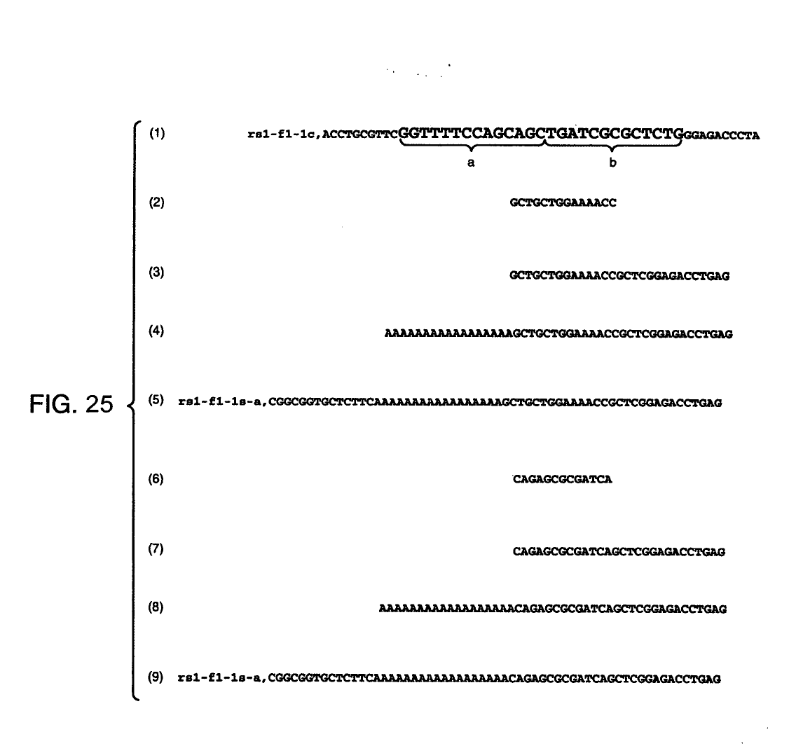

FIG. 25 diagrammatically shows generation of selection oligonucleotides, and uses construction oligonucleotide rs1-f1-1c (FIG. 24) as an example. For each construction oligonucleotide, two selection oligonucleotides (an “a” and a “b”) are generated. In FIG. 25, the portion of rs1-f1-1c exclusive of the sense5endAddOn and sense3endAddOn sequences is highlighted with a larger font at step (1). The program determines the “a” and “b” sections based on the specified value of selectionChip™ (bracket 126, FIG. 9B). In particular, the program identifies portions of the left and right sides of the construction oligonucleotide having a Tₘ closest to the specified selectionChip™ value. The “a” selection oligonucleotide (rs 1-f1-1s-a) is then generated by taking the complement of the “a” portion (step (2)), adding the sequence specified by the “selection3endAddOn” parameter (FIG. 9B) to the 3′ end of the complement (step (3)), adding sufficient adenine bases so that rs1-f1-1-s-a will have 60 bases (the number of bases being determined based on the selectionChip™ parameter) when the sequence specified by the “selection5endAddOn” parameter (FIG. 9B) is added (step (4)), and then adding the selection5endAddOn sequence (step (5)). The procedure is followed in steps (6) through (9) to obtain selection oligonucleotide rs1-f1-1-s-b. Similar steps are then followed to obtain “a” and “b” selection oligonucleotides for all construction oligonucleotides.

In block 38 (FIG. 7), the program then designs gene fragments and end primers. In particular, the program determines the length(s) of gene fragments to be synthesized as a function of the construction oligonucleotides. Using the “poolSize” input parameter (FIG. 9B), the program determines how many construction oligonucleotides can be used for each fragment. If poolSize=50, for example, up to 50 construction oligonucleotides can be used for each fragment. If poolSize is greater than or equal to the number of construction oligonucleotides designed for a sequence, the sequence can be synthesized as a single gene fragment, and a single set of left and right primers can be designed for that fragment. If poolSize is less than the number of construction oligonucleotides designed for a sequence, the sequence must be synthesized as multiple gene fragments, with each fragment having its own set of left and right primers.

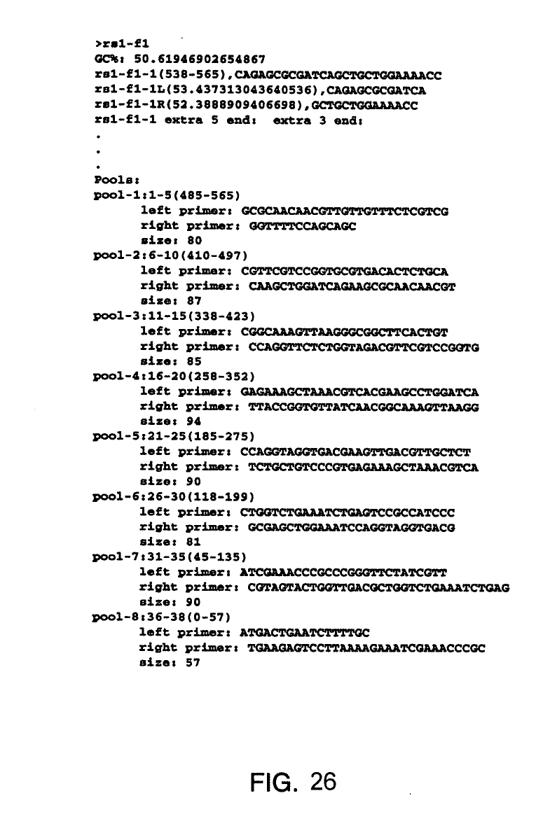

FIG. 18 shows a portion of an info.out file for rs1-f1, with poolSize=50 and pickSequenceBy=tm. Because this results in 38 construction oligonucleotides for rs1-f1 (i.e., a construction oligonucleotide corresponds to each of rs-f1-1 through rs1-f1-38), rs1-f1 can be synthesized as a single sequence. End primers are then designed for rs1-f1 by selecting enough bases at each end of the gene fragment so that the 5′ and 3′ primers have a melting point within a predetermined range of Oligo™. FIG. 26 shows a portion of an info.out file for rs1-f1, with poolSize=5 and pickSequenceBy=tm. In the case, the 38 construction oligonucleotides for rs1-f1 are divided into 8 “pools,” and rs1-f1 is synthesized as eight gene fragments. End primers are then designed for each of those eight fragment by selecting enough bases at each end of the gene fragment so that the 5′ and 3′ primers have a melting point within a predetermined range of oligo™.

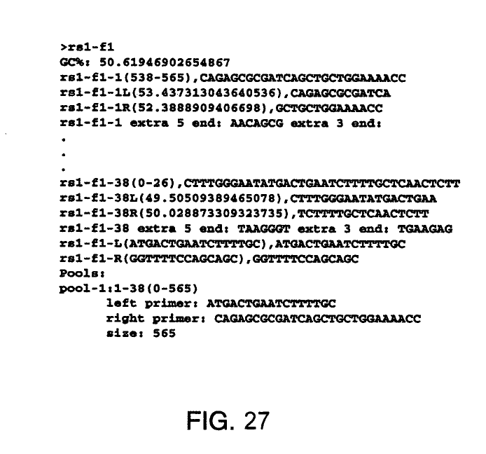

FIG. 27 is an example of an info.out file for rs1-f1, with poolSize=50, pickSequenceBy=tm and chipExtraSeqLen=7. In this case, the 7-base long sticky ends of the fragments are identified as the “extra 5 end[s]” and the “extra 3 end[s].”

From block 38 (FIG. 7), the program proceeds to block 40 and outputs files containing data for the designed construction and selection oligonucleotides. In addition to the “info.out” and “chipProduction” out files previously discussed, the program outputs two files listing the selection oligonucleotides (“chipSelectionA” and “chipSelectionB,” not shown), a file containing the input sequence(s) as divided at block 28 (“full_sequences.out,” as shown in FIG. 12), and a file containing oligonucleotides sequences that have reverse complementarity to the construction oligonucleotides.

This invention is further illustrated by the following examples, which should not be construed as limiting. The contents of all references, patents and published patent applications cited throughout this application are hereby incorporated by reference in their entirety for all purposes.

### EXAMPLE I

### Pre-Amplification

One or more oligonucleotides could be flanked by “temporary-tags” or “amplification sites” (e.g., universal temporary-tags, or universal amplification sites) that could be 5 to 30 bases long and/or could be lengthened during amplification cycles by having longer primers complementary to the tags at their 3′ ends. The primers would have 3′ terminal labile nucleotides, e.g. purines alkylated at their N7 position (N7me-dGTP). These would be heat labile and/or light labile and would last only a few rounds of PCR. When released or damaged, the next round of polymerase action would terminate at or near that position such that a “long-primer” appropriate for priming on an oligo or extended oligo (which is adjacent in the desired final sequence) is generated. Without intending to be bound by theory, this should work even if the chosen template is still flanked by temporary-tags. The very terminal tags are not labile and hence dominate in the final rounds. One way to synthesize the desired primers is extending with dimethylsulfate treated dATP or dGTP (and purified) on a template that has at its 5′ end the complementary extra nucleotide. An attractive alternative is to use one or more rNTPs at the 3′ end of the template primer. These would be destabilized by heat and Mg⁺⁺ or by RNAse. RnaseH is particularly suitable since it would preferentially hit the extended primers not the reserves; it can top some extent regenerate the original primer while creating a correctly truncated template.

Other variations on temporary tags include type-IIS restriction cleaving of the temporary-tags (set forth below), as well as or chemical cleavage requiring access to the reactions during the amplification.

### EXAMPLE II

### Recursive PCR Assembly Using Type-IIS Restriction Sites

Recursive PCR assembly of 38 pre-amplified 40-mers selected from a pool of 516 70-mers on a Xeotron-type chip, 14 to 28 base pair overlap. The two IIS enzymes chosen were:

5′ . . . G G T C T C (N)1e,cir . . . 3′BsaI
3′ . . . C C A G A G (N)5e,cir . . . 5′
5′ . . . A C C T G C (N)4e,cir . . . 3′BfuAI
3′ . . . T G G A C G (N)8e,cir . . . 5′

The strategy is set forth in Example IX.

### EXAMPLE III

### Use of the Same 7-mer Tag on Both Ends of A 44-mer (A 30-mer After Release)

Universal temp-primer: 5′ tagtaga 3′ (3′ underlined base is easily cleavable)

The temp-PCR product from rs1-1 is:

(SEQ ID NO:64)

5′ tagtagaTAAACAGGAAGATGCAAATTTTAGTAATAtctatcta 3′

(SEQ ID NO:65)

3′ atcatctATTTGTCCTTCTACGTTTAAAATCATTATagatagat 5′

After cleavage at the lower strand's special base and extension with the 7-mer we obtain the ss-37-mer below:

| | |
|---|---|
| 5′ tagtagaTAAACAGGAAGATGCAAATTTTAGTAATAA 3′ | (SEQ ID NO:66) |
| \|\|\|\|\|\|\| | |
| 3′ atcatctCATTATTAACGTTACCGTCTTCGTAAATTTCagatagat 5′ | (SEQ ID NO:67) |

which will pair with an overlapping 43-mer above (30-non-tag bases).

Two extensions later, the following ds-68-mer (54 non-tag bases) is generated:

(SEQ ID NO:68)

5′ tagtagaTAAACAGGAAGATGCAAATTTTAGTAATAATGCAATGGCA
GAAGCATTTAAAGtctatcta

(SEQ ID NO:69)

3″ atcatctATTTGTCCTTCTACGTTTAAAATCATTATTACGTTACCGT
CTTCGTAAATTTCagatagat

### EXAMPLE IV

### Using the Immobilized Synthesis Pattern to Bias the Order of Addition of Adjacent Oligos

If the genes are synthesized as clusters of oligonucleotides in the 2D layout, then they could be assembled in a manner similar to “in situ” polonies (i.e., polymerase colonies). The templates could be immobile 70-mers and the mobile phase (e.g., in a gel or polymer medium) would be universal primers and their extension products. Site-specific recombination points could be engineered for assembly of genes into larger chromosomes or in situ. Without intending to be bound be theory, this patterned assembly would greatly reduce problems of mispriming/misassembly since the number of choices are very small at each step. Another benefit is that the local concentrations are higher than if the entire mix were released into typical PCR reaction volumes (e.g., femtoliter polony scale reactions vs. microliter scale). For example, current Xeotron arrays synthesize 8000 oligonucleotides in a 20 nl volume. If these are diluted into typical PCR volumes (10 microliters) the concentrations are 1 pM of each oligo (=6 M molecules). PCR primers are typically used at 1000 nM, so even the undiluted 1 nM concentration is expected to go about a 1000 times more slowly at first (a bimolecular reaction, with one of the two molecules more dilute than usual).

A non-limiting example is the 2D array layout below, wherein the 4 primer pairs (e.g. 70-mer pair ab and bc) would extend on each other first (see dashes, producing abc, cde, efg and ghi), then because of extension and diffusion, two pairs of these products will coextend (along the vertical lines) to make abcde and efghi. Finally, these fuse to make the desired abcedefghi. The distance between the centers of the spots for each original pair might be 40 microns and 5 microns between closest points, while the centroids of the first pairs from the next pair might be 100 microns and 200 to the next etc.

ab-bc ef-fg
|-----|
cd-de gh-hi

### EXAMPLE V

### Post-Amplification Strand-Selection Strategy

An alternative is to alternate the strands synthesized (e.g. rs3-2 and the other even-numbered oligos in Example IX would be the reverse-complement of the one illustrated in example II above). Two PCR reactions would be made from the original chip pool. One would use a biotinylated L-primer that is cut with only BfuAI. This pool will be bound to Streptavidin-beads and the unbiotinylated strand can be released leaving ss-55-mers. The other reaction will use two unbiotinylated primers that are cut with both enzymes, releasing double stranded 40-mers. Only one strand of the 40-mers should bind to the 55-mer beads with 40-base-pair perfect matches. The overlaps of 14 to 28 bp should not bind significantly. Imperfect matches can be washed off at just less than the melting temperature (Tₘ), and perfect matches eluted at just over the Tₘ.

Software could be used to generate similar Tₘ points by varying the position of the 40-mers (or if size-selection can be relaxed, then length variation of the “40-mers” to 39, 41, etc. can make the Tₘ equalization better).

### EXAMPLE VI

### Pre-Amplification Plus Ligation Strategy

Ligations are typically performed at 1 nM concentration or higher. As one uses smaller (hence less expensive, e.g., Xeo-chips 8000*40/$2000=160 bases/$ vs. Illumina 6 bases/$) array elements, the amount of each oligomer decreases (at 4000 70-mer sequences per chip, this is about 1 fmol, reduced to 10% with capillary electrophoretic cleanup)=0.1 fmole of each oligo in 10 microliter ligation reaction=0.01 nM. The bimolecular reaction rate is thus expected to be slower by at least the square of the dilution factor (( 1/0.01)²=10,000 times slower). Including shared tag primers at the ends of each chip oligomer (e.g. 70-mer) allows PCR amplification. This should help recursive PCR as well, since the initial extension reactions depend on the same bimolecular (square law) interactions. The usual escape from this offered by PCR is not applicable since it requires driving the reaction with excess of both end primers which can't happen until the rare middle reactions occur. The combination of ligation and recursive PCR in principle help reduce the number of PCR cycles (e.g. by at least six cycles in the example 1 above, since 2ˆ6>38), but in practice those extra cycles need to be done anyway to get the amounts of DNA needed. The ligation can also select against mismatches at the 5′ and 3′ ends, but recursive PCR will do the same. Even if no theoretical advantage for ligation is evident, the empirical combination may win in some cases.

### EXAMPLE VII

### Integrated Multiplex Size, Mismatch and Open Reading Frame Selection Strategies

Size Selection

If all of the chip-oligomers have the same (or similar sizes), then the entire pool (or subset) can be multiplex-size-selected, e.g. by capillary electrophoresis or HPLC (before and/or after amplification). Similarly, if the ligation or Recursive PCR products have similar sizes, then multiplex-size-selection can be applied. The design of universal-gene-flanking PCR primers into the terminal oligos for each gene (or fragment) is often desirable and would not prevent use of gene-specific primers as well. If the DNAs have distinct sizes, these properties can be used to begin de-multiplexing (separation) at any stage.

Mismatch Selection

Method 1: The strand selection in Example V above can also be used to select against mismatches by pre-eluting just below the Tₘ of the pool. Software programs can be used to design the pool to be fairly homogeneous in Tₘ, if necessary, making separate chips for two or more Tₘ pools then pooling the pools after Tₘ selection. In order to maximize mismatch discrimination and to reduce conflict between size-uniformity and Tₘ-uniformity, one or more “selection-oligo-set” can be synthesized and amplified as above but with shorter overlaps with the main pool (e.g., sequential selection with two immobilized 24-mers (plus tags) rather than one 40-mer-plus-tag).

It has been determined that sequential rounds of hybridization selection are capable of reducing the chemical synthesis errors multiplicatively. It has been observed that error rates dropped from 1/160 in assemblies without selection to 1/1400 bp in assemblies using two sequential steps with overlapping ˜26-mer “selection” oligos covering the ˜50 bp “construction” oligos. The selection and construction lengths could vary but Tₘ could be brought as close to uniformity as desired by varying the lengths of the selection oligos at either end.

Method 2: MutS-protein-based selection.

Method 3: Homologous recombination in vivo or in vitro among double stranded and/or single stranded fragments.

Method 4: Randomly nicked and re-annealed pools are extended by DNA polymerase preferentially when the 3′ end matches the complementary template.

ORF Selection

Assembled genes (or intermediate fragments) can be selected in vivo (Lutz et al. (2002) Protein Eng. 15:1025) or in vitro (Jermutus et al. (2001) Proc. Natl. Acad. Sci. U.S.A. 98:75, incorporated herein by reference in its entirety for all purposes) to maintain reading frame (e.g., to overcome frame shift and nonsense mutations).

For any of the above selection methods, an optimal number of multiple rounds of selection can be employed to increase fidelity of the final product.

### EXAMPLE VIII

### Well Plate DNA Pool Sets

According to this embodiment of the invention, standard well plates, such as a universal 384-well plate, can be used in combination with the pool synthesis methods described herein and other methods of synthesizing large numbers of high-fidelity (or controlled diversity) DNAs to advantageously provide a platform for distribution and use of the DNAs.

One embodiment directed to synthetic genes recognizes that there are an increasing number of RNA and protein encoding genes in databases and increasing desire to use them singly and in various combinations, but the cost of storage, duplication and distribution can be prohibitive. According to the present invention, one standardized 384 well plate is used to collect and provide access to DNA samples including for example a collection of all human genes, numerous genes from plants, microbes, and viruses, many observed and theoretical splice variants, common mutant variants, codon-optimized versions, etc. easily totaling in the millions.

As an example, 884,736 (=96*96*96) genes can be made for as little as $35,000 per 50 chips (700,000 50-mers/Kbp/gene=17,500 genes per chip) as described above. Once the master plate is made, additional 384-well plates could be replicated for about $300 each (including PCR, primers, labor and infrastructure amortization). Each of these genes would be flanked by a nested set of three primer pairs. According to the invention, 288 universal primer pairs are used to access any amount of any gene. This gives a broad set of users access to a variety of genes or gene segments without cDNA cloning or individual stocking costs.

For illustrative purposes only, each of the genes has a representative structure as follows:

CCCCBBBBAAAAGGGGaaaabbbbccccpppp

In the above, aaaa and AAAA are the inner primer pair. The sequence of aaaa can be any sequence suitable for PCR priming (e.g. a 25-mer chosen to be far from the other primers) and can be unrelated to AAAA. BBBB and bbbb are the secondary pair, CCCC and cccc the outermost pair, and GGGG the desired gene.

A standard well plate, such as a 384 well plate is divided into quadrants with the upper left containing 96 sub-pools each containing 9216 (=96*96) genes each (already amplified by the outermost primers, CCCC/cccc). The lower left quadrant contains those 96 primer pairs in sufficient quantities to reamplify any/all of the above 96 pools. The upper right quadrant contains all of the secondary primers (BBBB & bbbb type) and the lower right the innermost primers (AAAA/aaaa). Any gene can be amplified by taking the appropriate well from the upper left quadrant, combining it with the appropriate primer pair in the upper right and PCR. Then a second PCR (optionally a cleanup step between PCRs) using the correct well from the lower right. The final product would be flanked by one of the AAAA/aaaa pairs which could contain signal for subsequent cleavage, expression, ligation, annealing, or binding for convenience in downstream applications.

According to an alternate embodiment, there are estimated to be well over 300,000 human exons and other important conserved elements in the human genome which might be reasonable segments for “targeted” sequencing in genetic association studies (e.g. where genetic variations in affected cases are compared with the same sites in controls). Even with very inexpensive DNA sequencing, a need exists to develop and use assays for genome subsets (e.g. frequent cancer genome surveillance and profiling).

According to this embodiment of the present invention, the protocol in this Example VIII is carried out, but replacing the genes with primers. 288 Universal primer pairs are used to access any amount of any primer pair. The result is a method of multiplex-testing and distributing large primer sets for case/control sequencing, which according to one specific embodiment may be carried out one 384 well plate.

### REFERENCES

Prodromou and Pearl (1992) Protein Eng. 5:827
Dillon, P. J. and Rosen, C. A. (1993) In White, B. A. (ed.), PCR Protocols: Current Methods and Applications. Humana Press, Totowa, N.J., Vol. 15, pp. 263-267.
Sardana et al. (1996) Plant Cell Rep. 15:677
Stemmer (1994) Proc. Natl. Acad. Sci. U.S.A. 91:10747
Ho et al. (1989) Gene 77:51

Each reference is incorporated herein by reference in its entirety for all purposes.

### EXAMPLE IX

### E. Coli Small Ribosomal Subunit

The following three genes (rs1, rs3 and rs14) were optimized for expression in E. coli.

Gene rs1, optimized for expression in E. coli extract:

(SEQ ID NO:7)

ATGACCGAATCATTCGCACAGTTATTCGAGGAAAGTTTAAAAGAAATTGA
AACCCGTCCGGGCTCAATCGTGCGTGGCGTAGTTGTTGCTATAGACAAAG
ATGTTGTTTTAGTTGATGCAGGTTTAAAAAGTGAAAGTGCAATTCCGGCA
GAACAGTTTAAAAATGCACAGGGTGAATTAGAAATTCAGGTAGGCGATGA
GGTAGATGTAGCTTTAGATGCAGTAGAGGATGGCTTCGGTGAAACCTTAT
TAAGTCGTGAAAAAGCAAAACGTCATGAAGCATGGATTACCTTAGAAAAA
GCATATGAAGATGCAGAAACTGTAACCGGTGTAATCAACGGCAAGGTAAA
AGGCGGCTTTACTGTTGAGTTAAATGGTATTCGTGCATTTTTACCAGGCA
GTTTAGTTGATGTTCGTCCGGTTCGTGATACCTTACATTTAGAAGGTAAA
GAATTAGAATTTAAAGTAATCAAATTAGATCAGAAACGTAACAACGTAGT
AGTTAGTCGTCGTGCAGTAATCGAAAGTGAAAACTCAGCAGAACGTGATC
AGTTATTAGAAAATCTGCAAGAAGGTATGGAAGTAAAGGGTATTGTAAAG
AATTTAACCGATTATGGTGCATTTGTCGACTTAGGCGGCGTTGATGGTTT
ATTACACATCACCGACATGGCATGGAAACGTGTTAAACATCCGAGTGAAA
TCGTAAATGTTGGCGACGAGATAACCGTAAAGGTTTTAAAATTTGATCGT
GAACGTACCCGTGTTAGTTTAGGATTGAAACAGTTAGGTGAAGATCCGTG
GGTTGCAATTGCAAAACGTTATCCGGAAGGTACCAAATTAACCGGCAGAG
TTACCAATTTAACCGATTATGGTTGCTTCGTAGAGATCGAGGAAGGTGTA
GAGGGCCTTGTTCACGTTAGTGAAATGGACTGGACCAATAAAAACATCCA
TCCGAGTAAAGTAGTAAACGTAGGTGACGTAGTGGAGGTAATGGTTTTAG
ATATCGACGAAGAACGTCGTCGTATTAGTTTAGGTTTAAAACAGTGCAAG
GCTAACCCGTGGCAGCAGTTCGCTGAAACCCATAATAAAGGCGACCGTGT
AGAGGGTAAGATTAAAAGCATTACTGACTTTGGCATCTTTATCGGCCTTG
ACGGTGGCATCGATGGTCTTGTCCATTTAAGTGACATCAGTTGGAATGTT
GCAGGTGAAGAAGCTGTACGTGAATATAAAAAAGGAGACGAAATTGCAGC
AGTTGTTTTACAGGTAGACGCAGAACGTGAACGTATTAGTCTGGGCGTAA
AGCAACTGGCAGAAGACCCGTTTAACAATTGGGTAGCTTTAAATAAAAAA
GGTGCAATTGTTACCGGTAAAGTTACCGCAGTAGACGCAAAAGGTGCAAC
TGTAGAACTGGCTGACGGCGTTGAAGGCTACTTACGTGCAAGTGAAGCAA
GTCGTGATCGTGTTGAAGATGCAACCCTTGTCTTAAGTGTAGGCGATGAA
GTTGAAGCAAAATTTACCGGTGTAGACCGTAAAAATCGTGCAATTAGTTT
AAGTGTTCGTGCAAAAGATGAAGCAGATGAAAAAGATGCAATTGCAACCG
TTAATAAACAGGAAGATGCAAATTTTAGTAATAATGCAATGGCAGAAGCA
TTTAAAGCAGCAAAAGGTGAATAA

Gene rs3, optimized for expression in E. coli extract:

(SEQ ID NO:70)

ATGGGACAGAAAGTTCATCCGAACGGCATTCGTCTGGGCATCGTAAAGCC
TTGGAATAGTACCTGGTTCGCTAATACCAAAGAATTTGCAGATAATCTGG
ACAGTGACTTCAAAGTTCGTCAGTATTTAACCAAAGAACTGGCTAAAGCA
AGTGTTAGTCGTATTGTTATTGAACGTCCGGCAAAAAGTATTCGTGTTAC
CATTCATACCGCACGTCCGGGAATAGTTATTGGTAAAAAAGGTGAAGACG
TAGAAAAATTACGTAAAGTTGTTGCAGACATAGCAGGCGTACCGGCACAG
ATTAATATTGCAGAAGTTCGTAAACCGGAATTAGATGCAAAACTTGTCGC
AGATAGTATTACCAGTCAGTTAGAAAGAAGAGTTATGTTCCGTCGTGCAA
TGAAGAGAGCAGTTCAGAACGCTATGCGTTTAGGTGCAAAAGGTATTAAA
GTTGAAGTTAGTGGTCGTTTAGGTGGTGCAGAAATTGCACGTACCGAATG
GTATCGTGAAGGTCGTGTTCCGTTACATACCTTACGTGCAGATATTGATT
ATAACACAAGTGAAGCACACACTACCTATGGCGTAATTGGTGTTAAGGTA
TGGATTTTCAAGGGTGAAATTTTAGGTGGTATGGCAGCAGTTGAACAGCC
GGAAAAACCGGCAGCACAGCCGAAAAAACAGCAGCGTAAAGGTCGTAAAT
AA

Gene rs14, optimized for expression in E. coli extract:

(SEQ ID NO:71)
+TL,1ATGGCAAAACAGTCAATGAAAGCTAGAGAAGTTAAACGTGTTGCATTAGC
AGATAAATATTTCGCTAAACGTGCAGAATTAAAAGCAATCATCTCAGACG
TTAATGCATCAGACGAAGATCGTTGGAACGCAGTTTTAAAATTACAGACC
TTACCGCGTGACTCAAGTCCGAGTCGTCAGCGTAACAGATGTCGTCAGAC
CGGCAGACCGCATGGCTTCTTACGTAAATTCGGCTTAAGTAGAATCAAAG
TTCGTGAAGCAGCAATGCGTGGTGAAATTCCGGGTTTAAAAAAAGCAAGT
TGGTAA

Oligonucleotides derived from sequences rs1, rs3, and rs14:

(SEQ ID NO:72)

rs1-1:
TAAACAGGAAGATGCAAATTTTAGTAATAATGCAATGGCAGAAGCATTTA
AAGCAGCAAAAGGTGAATAA

(SEQ ID NO:73)

rs 1-2:
AGATGAAGCAGATGAAAAAGATGCAATTGCAACCGTTAATAAACAGGAAG
ATGCAAATTTTAGTAATAAT

(SEQ ID NO:74)

rs 1-3:
GGTGTAGACCGTAAAAATCGTGCAATTAGTTTAAGTGTTCGTGCAAAAGA
TGAAGCAGATGAAAAAGATG

(SEQ ID NO:75)

rs1-4:
GCAACCCTTGTCTTAAGTGTAGGCGATGAAGTTGAAGCAAAATTTACCGG
TGTAGACCGTAAAAATCGTG

(SEQ ID NO:76)

rs1-5:
AAGGCTACTTACGTGCAAGTGAAGCAAGTCGTGATCGTGTTGAAGATGCA
ACCCTTGTCTTAAGTGTAGG

(SEQ ID NO:77)

rs1-6:
CGCAGTAGACGCAAAAGGTGCAACTGTAGAACTGGCTGACGGCGTTGAAG
GCTACTTACGTGCAAGTGAA

(SEQ ID NO:78)

rs1-7:
CAATTGGGTAGCTTTAAATAAAAAAGGTGCAATTGTTACCGGTAAAGTTA
CCGCAGTAGACGCAAAAGGT

(SEQ ID NO:79)

rs1-8:
TATTAGTCTGGGCGTAAAGCAACTGGCAGAAGACCCGTTTAACAATTGGG
TAGCTTTAAATAAAAAAGGT

(SEQ ID NO:80)

rs1-9:
ACGAAATTGCAGCAGTTGTTTTACAGGTAGACGCAGAACGTGAACGTATT
AGTCTGGGCGTAAAGCAACT

(SEQ ID NO:81)

rs1-10:
AGTTGGAATGTTGCAGGTGAAGAAGCTGTACGTGAATATAAAAAAGGAGA
CGAAATTGCAGCAGTTGTTT

(SEQ ID NO:82)

rs1-11:
TTATCGGCCTTGACGGTGGCATCGATGGTCTTGTCCATTTAAGTGACATC
AGTTGGAATGTTGCAGGTGA

(SEQ ID NO:83)

rs1-12:
TAAAGGCGACCGTGTAGAGGGTAAGATTAAAAGCATTACTGACTTTGGCA
TCTTTATCGGCCTTGACGGT

(SEQ ID NO:84)

rs1-13:
TTAAAACAGTGCAAGGCTAACCCGTGGCAGCAGTTCGCTGAAACCCATAA
TAAAGGCGACCGTGTAGAGG

(SEQ ID NO:85)

rs1-14:
GTAATGGTTTTAGATATCGACGAAGAACGTCGTCGTATTAGTTTAGGTTT
AAAACAGTGCAAGGCTAACC

(SEQ ID NO:86)

rs1-15:
ATCCATCCGAGTAAAGTAGTAAACGTAGGTGACGTAGTGGAGGTAATGGT
TTTAGATATCGACGAAGAAC

(SEQ ID NO:87)

rs1-16:
GGCCTTGTTCACGTTAGTGAAATGGACTGGACCAATAAAAACATCCATCC
GAGTAAAGTAGTAAACGTAG

(SEQ ID NO:88)

rs1-17:
CAATTTAACCGATTATGGTTGCTTCGTAGAGATCGAGGAAGGTGTAGAGG
GCCTTGTTCACGTTAGTGAA

(SEQ ID NO:89)

rs1-18:
TTGCAAAACGTTATCCGGAAGGTACCAAATTAACCGGCAGAGTTACCAAT
TTAACCGATTATGGTTGCTT

(SEQ ID NO:90)

rs1-19:
CCCGTGTTAGTTTAGGATTGAAACAGTTAGGTGAAGATCCGTGGGTTGCA
ATTGCAAAACGTTATCCGGA

(SEQ ID NO:91)

rs1-20:
GGCGACGAGATAACCGTAAAGGTTTTAAAATTTGATCGTGAACGTACCCG
TGTTAGTTTAGGATTGAAAC

(SEQ ID NO:92)

rs1-21:
CCGACATGGCATGGAAACGTGTTAAACATCCGAGTGAAATCGTAAATGTT
GGCGACGAGATAACCGTAAA

(SEQ ID NO:93)

rs1-22:
CCGATTATGGTGCATTTGTCGACTTAGGCGGCGTTGATGGTTTATTACAC
ATCACCGACATGGCATGGAA

(SEQ ID NO:94)

rs1-23:
AGAAAATCTGCAAGAAGGTATGGAAGTAAAGGGTATTGTAAAGAATTTAA
CCGATTATGGTGCATTTGTC

(SEQ ID NO:95)

rs1-24:
GTGCAGTAATCGAAAGTGAAAACTCAGCAGAACGTGATCAGTTATTAGAA
AATCTGCAAGAAGGTATGGA

(SEQ ID NO:96)

rs1-25:
TAATCAAATTAGATCAGAAACGTAACAACGTAGTAGTTAGTCGTCGTGCA
GTAATCGAAAGTGAAAACTC

(SEQ ID NO:97)

rs1-26:
TGATACCTTACATTTAGAAGGTAAAGAATTAGAATTTAAAGTAATCAAAT
TAGATCAGAAACGTAACAAC

(SEQ ID NO:98)

rs1-27:
TTACCAGGCAGTTTAGTTGATGTTCGTCCGGTTCGTGATACCTTACATTT
AGAAGGTAAAGAATTAGAAT

(SEQ ID NO:99)

rs1-28:
GTAAAAGGCGGCTTTACTGTTGAGTTAAATGGTATTCGTGCATTTTTACC
AGGCAGTTTAGTTGATGTTC

(SEQ ID NO:100)

rs1-29:
AAAGCATATGAAGATGCAGAAACTGTAACCGGTGTAATCAACGGCAAGGT
AAAAGGCGGCTTTACTGTTG

(SEQ ID NO:101)

rs1-30:
AAGTCGTGAAAAAGCAAAACGTCATGAAGCATGGATTACCTTAGAAAAAG
CATATGAAGATGCAGAAACT

(SEQ ID NO:102)

rs1-31:
AGATGTAGCTTTAGATGCAGTAGAGGATGGCTTCGGTGAAACCTTATTAA
GTCGTGAAAAAGCAAAACGT

(SEQ ID NO:103)

rs1-32:
AAATGCACAGGGTGAATTAGAAATTCAGGTAGGCGATGAGGTAGATGTAG
CTTTAGATGCAGTAGAGGAT

(SEQ ID NO:104)

rs1-33:
ATGCAGGTTTAAAAAGTGAAAGTGCAATTCCGGCAGAACAGTTTAAAAAT
GCACAGGGTGAATTAGAAAT

(SEQ ID NO:105)

rs1-34:
GTGCGTGGCGTAGTTGTTGCTATAGACAAAGATGTTGTTTTAGTTGATGC
AGGTTTAAAAAGTGAAAGTG

(SEQ ID NO:106)

rs1-35:
ACAGTTATTCGAGGAAAGTTTAAAAGAAATTGAAACCCGTCCGGGCTCAA
TCGTGCGTGGCGTAGTTGTT

(SEQ ID NO:107)

rs1-36:
ATGACCGAATCATTCGCACAGTTATTCGAGGAAAGTTTAAAAGAAAT

(SEQ ID NO:108)

rs3-1:
GGCAGCAGTTGAACAGCCGGAAAAACCGGCAGCACAGCCGAAAAAACAGC
AGCGTAAAGGTCGTAAATAA

(SEQ ID NO:109)

rs3-2:
GGCGTAATTGGTGTTAAGGTATGGATTTTCAAGGGTGAAATTTTAGGTGG
TATGGCAGCAGTTGAACAGC

(SEQ ID NO:110)

rs3-3:
TACGTGCAGATATTGATTATAACACAAGTGAAGCACACACTACCTATGGC
GTAATTGGTGTTAAGGTATG

(SEQ ID NO:111)

rs3-4:
TACCGAATGGTATCGTGAAGGTCGTGTTCCGTTACATACCTTACGTGCAG
ATATTGATTATAACACAAGT

(SEQ ID NO:112)

rs3-5:
GTATTAAAGTTGAAGTTAGTGGTCGTTTAGGTGGTGCAGAAATTGCACGT
ACCGAATGGTATCGTGAAGG

(SEQ ID NO:113)

rs3-6:
AAGAGAGCAGTTCAGAACGCTATGCGTTTAGGTGCAAAAGGTATTAAAGT
TGAAGTTAGTGGTCGTTTAG

(SEQ ID NO:114)

rs3-7:
GTATTACCAGTCAGTTAGAAAGAAGAGTTATGTTCCGTCGTGCAATGAAG
AGAGCAGTTCAGAACGCTAT

(SEQ ID NO:115)

rs3-8:
GTAAACCGGAATTAGATGCAAAACTTGTCGCAGATAGTATTACCAGTCAG
TTAGAAAGAAGAGTTATGTT

(SEQ ID NO:116)

rs3-9:
CAGACATAGCAGGCGTACCGGCACAGATTAATATTGCAGAAGTTCGTAAA
CCGGAATTAGATGCAAAACT

(SEQ ID NO:117)

rs3-10:
TAGTTATTGGTAAAAAAGGTGAAGACGTAGAAAAATTACGTAAAGTTGTT
GCAGACATAGCAGGCGTACC

(SEQ ID NO:118)

rs3-11:
AAAAAGTATTCGTGTTACCATTCATACCGCACGTCCGGGAATAGTTATTG
GTAAAAAAGGTGAAGACGTA

(SEQ ID NO:119)

rs3-12:
GGCTAAAGCAAGTGTTAGTCGTATTGTTATTGAACGTCCGGCAAAAAGTA
TTCGTGTTACCATTCATACC

(SEQ ID NO:120)

rs3-13:
TCTGGACAGTGACTTCAAAGTTCGTCAGTATTTAACCAAAGAACTGGCTA
AAGCAAGTGTTAGTCGTATT

(SEQ ID NO:121)

rs3-14:
CCTTGGAATAGTACCTGGTTCGCTAATACCAAAGAATTTGCAGATAATCT
GGACAGTGACTTCAAAGTTC

(SEQ ID NO:122)

rs3-15:
ATGGGACAGAAAGTTCATCCGAACGGCATTCGTCTGGGCATCGTAAAGCC
TTGGAATAGTACCTGGTTCG

(SEQ ID NO:123)

rs3-16:
ATGGGACAGAAAGTTCATCC

(SEQ ID NO:124)

rs14-1:
AAGTAGAATCAAAGTTCGTGAAGCAGCAATGCGTGGTGAAATTCCGGGTT
TAAAAAAAGCAAGTTGGTAA

(SEQ ID NO:125)

rs14-2:
TCGTCAGACCGGCAGACCGCATGGCTTCTTACGTAAATTCGGCTTAAGTA
GAATCAAAGTTCGTGAAGCA

(SEQ ID NO:126)

rs14-3:
AAAATTACAGACCTTACCGCGTGACTCAAGTCCGAGTCGTCAGCGTAACA
GATGTCGTCAGACCGGCAGA

(SEQ ID NO:127)

rs14-4:
CATCTCAGACGTTAATGCATCAGACGAAGATCGTTGGAACGCAGTTTTAA
AATTACAGACCTTACCGCGT

(SEQ ID NO:128)

rs14-5:
GCATTAGCAGATAAATATTTCGCTAAACGTGCAGAATTAAAAGCAATCAT
CTCAGACGTTAATGCATCAG

(SEQ ID NO:129)

rs14-6:
ATGGCAAAACAGTCAATGAAAGCTAGAGAAGTTAAACGTGTTGCATTAGC
AGATAAATATTTCGCTAAAC

(SEQ ID NO:130)

rs14-7:
ATGGCAAAACAGTCAATGAAAG

Full list of 70-mers generated for rs3 gene. 15-mer tags are underlined and 6-mer IIS sites are bolded.

(SEQ ID NO:131)

rs3-1: CACTCCAGGGTCTCGTTATTTACGACCTTTACGCTGCTGTTTT
TTCGGCTGTGCTCGTCGCAGGTGTCAC

(SEQ ID NO:132)

rs3-2: CACTCCAGGGTCTCGCTGTTTTTTCGGCTGTGCTGCCGGTTTT
TCCGGCTGTTCACGTCGCAGGTGTCAC

(SEQ ID NO:133)

rs3-3: CACTCCAGGGTCTCGGTTTTTCCGGCTGTTCAACTGCTGCCATACCAC
CTAAAATCGTCGCAGGTGTCAC

(SEQ ID NO:134)

rs3-4: CACTCCAGGGTCTCGCTGCCATACCACCTAAAATTTCACCCTT
GAAAATCCATACCGTCGCAGGTGTCAC

(SEQ ID NO:135)

rs3-5: CACTCCAGGGTCTCGTCACCCTTGAAAATCCATACCTTAACAC
CAATTACGCCATCGTCGCAGGTGTCAC

(SEQ ID NO:136)

rs3-6: CACTCCAGGGTCTCGATACCTTAACACCAATTACGCCATAGGT
AGTGTGTGCTTCCGTCGCAGGTGTCAC

(SEQ ID NO:137)

rs3-7: CACTCCAGGGTCTCGGCCATAGGTAGTGTGTGCTTCACTTGTG
TTATAATCAATACGTCGCAGGTGTCAC

(SEQ ID NO:138)

rs3-8: CACTCCAGGGTCTCGTGCTTCACTTGTGTTATAATCAATATCT
GCACGTAAGGTACGTCGCAGGTGTCAC

(SEQ ID NO:139)

rs3-9: CACTCCAGGGTCTCGTATAATCAATATCTGCACGTAAGGTATG
TAACGGAACACGCGTCGCAGGTGTCAC

(SEQ ID NO:140)

rs3-10: CACTCCAGGGTCTCGGTAAGGTATGTAACGGAACACGACCTT
CACGATACCATTCCGTCGCAGGTGTCAC

(SEQ ID NO:141)

rs3-11: CACTCCAGGGTCTCGCGACCTTCACGATACCATTCGGTACGT
GCAATTTCTGCACCGTCGCAGGTGTCAC

(SEQ ID NO:142)

rs3-12: CACTCCAGGGTCTCGGGTACGTGCAATTTCTGCACCACCTAA
ACGACCACTAACTCGTCGCAGGTGTCAC

(SEQ ID NO:143)

rs3-13: CACTCCAGGGTCTCGCACCTAAACGACCACTAACTTCAACTT
TAATACCTTTTGCCGTCGCAGGTGTCAC

(SEQ ID NO:144)

rs3-14: CACTCCAGGGTCTCGCACTAACTTCAACTTTAATACCTTTTG
CACCTAAACGCATCGTCGCAGGTGTCAC

(SEQ ID NO:145)

rs3-15: CACTCCAGGGTCTCGAATACCTTTTGCACCTAAACGCATAGC
GTTCTGAACTGCTCGTCGCAGGTGTCAC

(SEQ ID NO:146)

rs3-16: CACTCCAGGGTCTCGGCATAGCGTTCTGAACTGCTCTCTTCA
TTGCACGACGGAACGTCGCAGGTGTCAC

(SEQ ID NO:147)

rs3-17: CACTCCAGGGTCTCGCTCTTCATTGCACGACGGAACATAACT
CTTCTTTCTAACTCGTCGCAGGTGTCAC

(SEQ ID NO:148)

rs3-18: CACTCCAGGGTCTCGGGAACATAACTCTTCTTTCTAACTGAC
TGGTAATACTATCCGTCGCAGGTGTCAC

(SEQ ID NO:149)

rs3-19: CACTCCAGGGTCTCGTCTTTCTAACTGACTGGTAATACTATC
TGCGACAAGTTTTCGTCGCAGGTGTCAC

(SEQ ID NO:150)

rs3-20: CACTCCAGGGTCTCGGTAATACTATCTGCGACAAGTTTTGCA
TCTAATTCCGGTTCGTCGCAGGTGTCAC

(SEQ ID NO:151)

rs3-21: CACTCCAGGGTCTCGAAGTTTTGCATCTAATTCCGGTTTACG
AACTTCTGCAATACGTCGCAGGTGTCAC

(SEQ ID NO:152)

rs3-22: CACTCCAGGGTCTCGGGTTTACGAACTTCTGCAATATTAATC
TGTGCCGGTACGCCGTCGCAGGTGTCAC

(SEQ ID NO:153)

rs3-23: CACTCCAGGGTCTCGATATTAATCTGTGCCGGTACGCCTGCT
ATGTCTGCAACAACGTCGCAGGTGTCAC

(SEQ ID NO:154)

rs3-24: CACTCCAGGGTCTCGCCTGCTATGTCTGCAACAACTTTACGT
AATTTTTCTACGTCGTCGCAGGTGTCAC

(SEQ ID NO: 155)

rs3-25: CACTCCAGGGTCTCGAACAACTTTACGTAATTTTTCTACGTC
TTCACCTTTTTTACGTCGCAGGTGTCAC

(SEQ ID NO:156)

rs3-26: CACTCCAGGGTCTCGTTTCTACGTCTTCACCTTTTTTACCAA
TAACTATTCCCGGCGTCGCAGGTGTCAC

(SEQ ID NO:157)

rs3-27: CACTCCAGGGTCTCGACCTTTTTTACCAATAACTATTCCCGG
ACGTGCGGTATGACGTCGCAGGTGTCAC

(SEQ ID NO:158)

rs3-28: CACTCCAGGGTCTCGGGACGTGCGGTATGAATGGTAACACGA
ATACTTTTTGCCGCGTCGCAGGTGTCAC

(SEQ ID NO:159)

rs3-29: CACTCCAGGGTCTCGATGGTAACACGAATACTTTTTGCCGGA
CGTTCAATAACAACGTCGCAGGTGTCAC

(SEQ ID NO:160)

rs3-30: CACTCCAGGGTCTCGCCGGACGTTCAATAACAATACGACTAA
CACTTGCTTTAGCCGTCGCAGGTGTCAC

(SEQ ID NO:161)

rs3-31: CACTCCAGGGTCTCGAATACGACTAACACTTGCTTTAGCCAG
TTCTTTGGTTAAACGTCGCAGGTGTCAC

(SEQ ID NO:162)

rs3-32: CACTCCAGGGTCTCGTTTAGCCAGTTCTTTGGTTAAATACTG
ACGAACTTTGAAGCGTCGCAGGTGTCAC

(SEQ ID NO:163)

rs3-33: CACTCCAGGGTCTCGGGTTAAATACTGACGAACTTTGAAGTC
ACTGTCCAGATTACGTCGCAGGTGTCAC

(SEQ ID NO:164)

rs3-34: CACTCCAGGGTCTCGTTTGAAGTCACTGTCCAGATTATCTGC
AAATTCTTTGGTACGTCGCAGGTGTCAC

(SEQ ID NO:165)

rs3-35: CACTCCAGGGTCTCGAGATTATCTGCAAATTCTTTGGTATTA
GCGAACCAGGTACCGTCGCAGGTGTCAC

(SEQ ID NO:166)

rs3-36: CACTCCAGGGTCTCGTGGTATTAGCGAACCAGGTACTATTCC
AAGGCTTTACGATCGTCGCAGGTGTCAC

(SEQ ID NO:167)

rs3-37: CACTCCAGGGTCTCGGTACTATTCCAAGGCTTTACGATGCCC
AGACGAATGCCGTCGTCGCAGGTGTCAC

(SEQ ID NO:168)

rs3-38: CACTCCAGGGTCTCGGCCCAGACGAATGCCGTTCGGATGAAC
TTTCTGTCCCATACGTCGCAGGTGTCAC

Two 15-mer “tag” pre-primers are used for PCR:

| | |
|---|---|
| L = 5′ CACTCCAGGGTCTCG | (SEQ ID NO:169) |
| R = 5′ GTGACACCTGCGACG | (SEQ ID NO:170) |

The double stranded 70-mer for the first oligonucleotide sequence (rs3-1) with nuclease breaks indicated by gaps:

(SEQ ID NO:171)
CACTCCAGGGTCTCG TTATTTACGACCTTTACGCTGCTGTTTTTTCGGCTG TGCTCGTCGCAGGTGTCAC
(SEQ ID NO:172)
gtgaggtcccagagcaata aatgctggaaatgcgacgacaaaaaagccgacacga gcagcgtccacagag

Eliminating tags and overlaps and reverse complement yields the following 708-mer:

(SEQ ID NO:63)

TATGGGACAGAAAGTTCATCCGAACGGCATTCGTCTGGGCATCGTAAAGC
CTTGGAATAGTACCTGGTTCGCTAATACCAAAGAATTTGCAGATAATCTG
GACAGTGACTTCAAAGTTCGTCAGTATTTAACCAAAGAACTGGCTAAAGC
AAGTGTTAGTCGTATTGTTATTGAACGTCCGGCAAAAAGTATTCGTGTTA
CCATTCATACCGCACGTCCGGGAATAGTTATTGGTAAAAAAGGTGAAGAC
GTAGAAAAATTACGTAAAGTTGTTGCAGACATAGCAGGCGTACCGGCACA
GATTAATATTGCAGAAGTTCGTAAACCGGAATTAGATGCAAAACTTGTCG
CAGATAGTATTACCAGTCAGTTAGAAAGAAGAGTTATGTTCCGTCGTGCA
ATGAAGAGAGCAGTTCAGAACGCTATGCGTTTAGGTGCAAAAGGTATTAA
AGTTGAAGTTAGTGGTCGTTTAGGTGGTGCAGAAATTGCACGTACCGAAT
GGTATCGTGAAGGTCGTGTTCCGTTACATACCTTACGTGCAGATATTGAT
TATAACACAAGTGAAGCACACACTACCTATGGCGTAATTGGTGTTAAGGT
ATGGATTTTCAAGGGTGAAATTTTAGGTGGTATGGCAGCAGTTGAACAGC
CGGAAAAACCGGCAGCACAGCACAGCCGAAAAAACAGCAGCGTAAAGGTC
GTAAATAA

The flanking primers used for the final PCR are:

| | |
|---|---|
| rs3-L = 5′ ATGGGACAGAAAGTTCATC | (SEQ ID NO:36) |
| rs3-R = 5′ TTATTTACGACCTTTACGCT | (SEQ ID NO:37) |

### EXAMPLE X

### Design of Sequences

Gene and oligonucleotide sequences were designed using a Java program, CAD-PAM. Basically, CAD-PAM uses constraints on the amino acid sequences, codon usage, messenger RNA secondary structure and restriction enzymes used to release the construction oligonucleotides in order to create nearly optimal, overlapping sets of n-mer (typically 50-mer) construction oligomers and shorter selection oligomers (typically 26-mer). The melting temperatures (Tₘ) of overlapping regions between adjacent gene construction oligonucleotides or between construction and selection oligonucleotides were equalized. The selection oligonucleotides were padded with extra adenine residues to keep oligomer length constant (70-mers) for optional size selection (not used for typical PAM). Tₘ values were calculated using the nearest neighbor method (Breslauer et al. (1986) Proc. Natl. Acad. Sci. USA. 83:3746, incorporated by reference herein in its entirety for all purposes). Codons can be fixed or altered to allow expression improvements.

### EXAMPLE XI

### Amplification of Synthesized Oligonucleotides

Current microchips have very low surface areas and therefore produce only small amounts of oligonucleotides. When released into solution, the oligonucleotides are present at picomolar or lower concentrations per sequence, concentrations that are insufficiently high to efficiently drive bimolecular priming reactions such as, for example, those involved in PCR assembly, ligation assembly, etc.

To address this problem of scale, oligonucleotides obtained from the microchips were amplified from roughly as little as 10⁵ (or 10⁹ for low density arrays) up to 10⁹ (or 10¹²) molecules of each sequence, thereby permitting subsequent selection and assembly steps. An overview of the integrated process is presented in FIG. 6.

For this amplification method, oligonucleotides flanked by universal primer sequences were synthesized on a programmable microchip. This generates a pool of 10²-10⁵ different oligonucleotides, which can be released from the microchips by chemical or enzymatic treatment. Released oligonucleotides were amplified by polymerase chain reaction (PCR) using primers that contained type-IIS restriction enzyme recognition sites. Digestion of the PCR products with the corresponding restriction enzyme(s) yielded sufficient amounts of unadulterated oligonucleotide sequences to be used for gene or genome assembly.

The feasibility of this approach was first demonstrated with Atactic/Xeotron 4K (that is, 3,968 synthesis chambers) photo-programmable microfluidic microarrays (Zhou, X. et al., Nucleic Acids Res. 32: 5409-5417 (2004)). To monitor oligonucleotide synthesis and cleavage from the microchip, the 5′ ends of the oligonucleotides were coupled with fluorescein. The microchip was scanned with a microarray scanner before and after cleavage. The cleaved portions of the oligonucleotides were hybridized onto a ‘quality-assessment (QA)-chip’ synthesized with complementary oligonucleotide sequences. These results demonstrated that individual oligonucleotides were synthesized and nearly completely released from the microchip in quantities that can be measured by a QA-chip hybridization process. The typical yield of oligonucleotide released from each chamber of the 4K microchip was about 5 fmoles, as determined by quantitative PCR (Zhou, X. et al. Nucleic Acids Res. 32: 5409-5417 (2004)). Using primers that annealed specifically to the universal primers flanking the oligonucleotide sequences, PCR reactions were carried out to amplify the oligonucleotides more than a million-fold.

### EXAMPLE XII

### Error Reduction of Synthesized and/or Amplified Oligonucleotides

Mutations incurred during oligonucleotide synthesis are a major source of errors in assembled DNA molecules, and are costly and difficult to eradicate (Cello et al. (2002) Science 297:1016; Smith et al. (2003) Proc. Natl. Acad. Sci. USA 100: 15440). This example describes a simple, stringent hybridization-based method to remove oligonucleotides with such mutations. To select against mutations in construction oligonucleotides, these oligonucleotides were hybridized sequentially to two pools of bead-immobilized short complementary selection oligonucleotides that together span the entire length of the construction oligonucleotides (FIG. 5). All selection oligonucleotides were designed to have nearly identical melting temperatures by varying their lengths. Under appropriate hybridization conditions, imperfect pairs between selection and construction oligonucleotides due to base-mismatch or deletion have lower melting temperatures and are unstable. After the cycles of hybridization, wash and elution, oligonucleotides with sequences that perfectly match the selection oligonucleotides were preferentially retained and enriched. Digestion of the PCR products with type-IIS restriction enzymes removed the generic primer sequences from both ends of the oligonucleotides. In these experiments the amplification tags were removed just before selection. However, if the digestion were deferred, the oligonucleotides could be re-amplified by PCR and subjected to further rounds of hybridization selection. Without intending to be bound by theory, because the probability of complementary mutations occurring at matching positions on construction and selection oligonucleotides is miniscule, in principle most oligonucleotides with mutations can be eliminated by this selection procedure.

Like construction oligonucleotides, selection oligonucleotides were also synthesized and released from programmable microarrays. Selection oligonucleotides with arms were amplified by PCR, and the strands complementary to the gene construction oligonucleotide were labeled with biotin at the 5′ end and selectively immobilized on streptavidin beads. The unlabelled strands were denatured and removed. Immobilized selection oligonucleotides selectively retained the correct 50-base pair construction oligonucleotides.

The error-reduced construction oligonucleotides are suitable for gene assembly. To facilitate automation, a single-step polymerase assembly multiplexing (PAM) reaction was developed for multiple gene syntheses from a single pool of oligonucleotides. Single-fragment assembly methods have traditionally used two or three steps (ligation, assembly and PCR) (Cello, J., et al., Science 297: 1016-1018 (2002); Smith, H. O. et al., Proc. Natl. Acad. Sci. USA 100: 15440-15445 (2003); Stemmer, W. P. et al., Gene 164: 49-53 (1995)). For PAM, gene-flanking primer pairs were added to the pool of gene-construction oligonucleotides (with the primer pairs at a higher concentration than the oligonucleotides), together with thermostable polymerase and dNTPs. Extension of overlapping oligonucleotides and subsequent amplification of multiple full-length genes were accomplished in a closed-tube, one-step reaction using a thermal cycler. Different generic adaptor sequences could be incorporated into the ends of each gene or gene set, and a set of complementary adaptor-primer pairs can be pre-synthesized to avoid the cost of synthesizing gene-specific PAM primer pairs and to facilitate automation (for example, 96 or 384 generic adaptors to match standard multi-well plates).

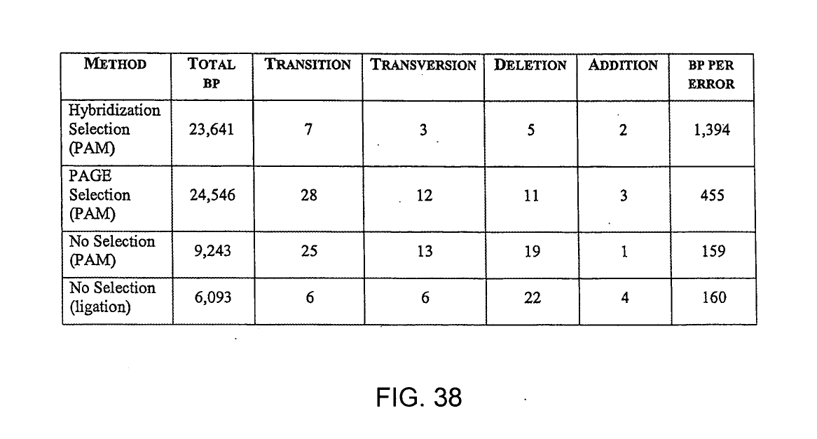

To determine the efficiency of the hybridization-selection method to eliminate mismatch mutations (Eason, R. G. et al. Proc. Natl. Acad. Sci. USA 101: 11046-11051 (2004)), genes were constructed using the same pool of microchip-synthesized oligonucleotides purified in three different ways: unpurified, polyacrylamide gel electrophoresis (PAGE)-purified or hybridization-purified. These genes were cloned and random clones from each category were sequenced in both directions to determine error types and rates for each category. As shown in (FIG. 38), genes synthesized with unpurified oligonucleotides have the highest error rates (1 in 160 bp); the method of gene assembly (using ligation or PAM) made little difference. PAGE purification of oligonucleotides reduced the error rate to 1 in 450 bp, mainly through removal of deletion mutations. This rate is comparable to figures reported by other groups using PAGE purification (Cello, J., et al., Science 297: 1016-1018 (2002); Smith, H. O. et al., Proc. Natl. Acad. Sci. USA 100: 15440-15445 (2003)). With hybridization selection, the error rate was further reduced to approximately 1 in 1,394 bp.

### EXAMPLE XII

### Parallel Assembly of Multiple Genes in a Single Pool

A microchip was used to redesign and synthesize codon-altered versions of the 21 protein-encoding genes that constitute the E. coli small ribosomal subunit. Translational efficiencies of the natural versions of these 21 proteins are very low in vitro, even though in vivo the proteins have high expression levels (Culver, G. M. & Noller, H. F. RNA 5: 832-843 (1999)). Redesigning codon usage is a way to increase protein translation efficiencies, although it is more challenging to accomplish when starting with nearly ideal codons. Because many other proteins are expressed well in this in vitro system, it was hypothesized that some of the problem was due to secondary structure (possibly exacerbated by the fact that the rate of T7 polymerase-mediated transcription is eightfold higher than translation) (lost, I., et al., J. Bacteriol. 174: 619-622 (1992); Iost, I. & Dreyfus, M. Nature 372: 193-196 (1994)). Codons were replaced with sequences likely to have less secondary structure (for example, by lowering G+C content). The CAD-PAM software (FIG. 7) designed overlapping 50-bp oligonucleotide sequences (embedded in 70-mers) for the 21 ribosomal genes and synthesized them all on a 4K Xeochip. These oligonucleotides were processed and hybridization-selected with selection oligonucleotides, and were then used to construct the 21 ribosomal genes in multiple PAM reactions. Error-free clones were tested in E. coli using coupled in vitro transcription-translation reactions. The translation profiles of the synthetic genes were determined. A number of codon-altered genes had higher translation levels in the E. coli extract compared with their respective wild-type genes. These 21 genes were combined using sequential PAM reactions to give a pool of ˜14.6 kb assemblies by introducing unique ˜30-mer overlapping linkers between gene units and performing sequential PAM reactions. Correct assembly was confirmed by sequencing on average four individual clones from every overlapping DNA segment generated by high-fidelity PCR reactions that together covered the whole construct. By starting with correct input gene sequences, and through repeated high-fidelity, polymerase-based extension reactions, the assembly process resulted in a lower error rate (about 1 in 7,300 bp) than any of the methods shown in FIG. 38 (all of which started with oligonucleotides containing synthetic errors). This clearly demonstrated that a major source of error for gene assembly comes from oligonucleotide chemical synthesis rather than polymerase proofreading activity. Although the increasing length of the PCR products might be expected to reduce yield in the later assemblies, the number of reaction components decreases and so the efficiency remains high. If PAM length does become limiting, homologous recombination may be used to allow assembly in the megabase range.

Several successful assembly reactions were carried out using the methods described herein. For example, the 14-kb operon of 21 ribosomal genes was assembled using polymerase assembly multiplexing as described herein. Production of the full length fragment was confirmed by gel electrophoresis. Additionally, the s19 gene was successfully assembled from a mixture of oligos from a Nimbelgen custom array of 95,376 oligos (6.7 megabases). The results were confirmed by gel electrophoresis.

### EXAMPLE XIV

### Methods for Examples XI-XII

Design of sequences

Gene and oligonucleotide sequences were designed using the Java program CAD-PAM as described further herein. Basically, CAD-PAM uses constraints on the amino acid sequences, codon usage, messenger RNA secondary structure and restriction enzymes used to release the construction oligonucleotides in order to create nearly optimal, overlapping sets of n-mer (typically 50-mer) construction oligomers and shorter selection oligomers (typically 26-mer). The melting temperatures (Tₘ) of overlapping regions between adjacent gene construction oligonucleotides or between construction and selection oligonucleotides were equalized. The selection oligonucleotides were padded with extra adenine residues to keep oligomer length constant (70-mers) for optional size selection (not used for typical PAM). Tₘ values were calculated using the nearest neighbor method (Breslauer, K. J., et al., Proc. Natl. Acad. Sci. USA 83: 3746-3750 (1986)). Codons can be fixed or altered to allow expression improvements.

Microchip Synthesis, Amplification and Selection of Oligonucleotides

Oligonucleotides were synthesized on photo-programmable microfluidic microchips with a phosphate at the 5′ end and the 3′ end coupling to the 3′-hydroxy terminus of a uracil residue. After synthesis, the oligonucleotides were cleaved either with RNase A or by ammonium hydroxide treatment (used for deprotection as in standard oligonucleotide syntheses) followed by precipitation. Gene construction oligonucleotides that had been PCR amplified with 20-mers (initially complementary to the terminal ten bases) were digested with the type-IIS restriction enzymes BsaI and BseRI (without gel purification except for the ‘PAGE’ controls). Immobilization of biotin-labeled selection oligonucleotides on magnetic streptavidin beads (Dynal Biotech, Brown Deer, Wis.) and removal of the non-biotinylated strand were done as described (Espelund, M., et al., Nucleic Acids Res. 18: 6157-6158 (1990)). Construction oligonucleotides were denatured at 95° C. for 3 min and hybridized to selection oligonucleotides in hybridization buffer (5×SSPET buffer, 50% formamide, 0.2 mg ml⁻¹ BSA) for 14-16 h at 42° C. on a rotor. Beads were washed three times with 0.5×SSPET and three times with wash buffer (20 mM Tris-HCl pH 7.0, 5 mM EDTA, 4 mM NaCl) at room temperature. The construction oligonucleotides were recovered by denaturation in 0.1 M NaOH for 15 min and subsequent neutralization.

Polymerase Assembly Multiplexing Reactions

PAM reactions were carried out in 25 μl reactions containing 2 μl of oligonucleotide mixtures, 0.4 μM of each of the gene-end primer pairs, 1×dNTP mixture and 0.5 μl of Advantage 2 polymerase mixture in 1× buffer (Clontech ADVANTAGE 2™ PCR kit). Samples were denatured at 95° C. for 3 min, then underwent 40-45 thermal cycles of 95° C. for 30 s, 49° C. for 1 min and 68° C. for 1 min kb⁻¹, then finished at 68° C. for 10 min. Sequential PAM reactions were used to combine multiple genes. First, His6-tagged linear expression constructs of the correct sequences of 21 ribosomal protein genes were pre-constructed by PCR using an RTS E. coli linear template generation kit (Roche). These constructs were then used as templates in separate PCR reactions where unique ˜30-mer linkers with identical Tₘ (0.4 μM of each, Integrated DNA Technologies, Inc.) were introduced to create enough overlapping sequences between genes for secondary PAM reactions. In these, three large fragments were made in separate Roche Expand long template PCR reactions: RS1-5 (1-5,513), RS6-13 (5,483-10,526) and RS14-21 (10,497-14,593). These fragments were gel-purified and assembled into a full 14,593-bp operon in the final assembly reaction using RS1-21 (1-14,593). For the last two assemblies, samples were denatured at 92° C. for 2 min, followed by 10 thermal cycles at 92° C. for 30 s, 65° C. for 1 min and 68° C. for 1 min kb⁻¹, then followed by 25 additional cycles at 92° C. for 30 s, 65° C. for 1 min, and 68° C. for 1 min kb⁻¹ plus 10 s per cycle, and finished at 68° C. for 10 min.

Coupled In Vitro Transcription and Translation

Assembled genes were cloned and error-free clones were selected by sequencing. Linear constructs for in vitro protein expression were made using Roche RTS E. coli linear template generation set, His-tag. In-vitro-coupled transcription and translation was performed using a Roche Rapid Translation System RTS 100 E. coli HY kit. Proteins were detected by western blotting with an anti-His6-peroxidase antibody (Roche) using standard procedures.

### EQUIVALENTS

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application were specifically indicated to be so incorporated by reference.

1-84. (canceled)

85. An article of manufacture comprising a multiplicity of different, retrievable polynucleotides, the article comprising:
a polynucleotide reservoir which contains a mixture of different polynucleotides comprising differing pairs of primer sequences which permit amplification of a subgroup of said different polynucleotides from said reservoir; and
plural primer reservoirs each of which contains a pair of oligonucleotide primers complementary to a pair of primer sequences of a polynucleotide in the construct reservoir.

86. The article of claim 85 wherein the primer sequence pairs of polynucleotides in a polynucleotide reservoir are different from each other.

87. The article of claim 85 wherein the polynucleotides comprise synthetic DNA.

88. The article of claim 85 wherein the polynucleotides comprise genes.

89. The article of claim 85 wherein the polynucleotides comprise multiple mutants of a wild-type sequence.

90. The article of claim 85 wherein the polynucleotides comprise vectors.

91. The article of claim 85 wherein at least a portion of said polynucleotides are at least one Kb long.

92. The article of claim 85 wherein at least a portion of said polynucleotides are at least two Kb long.

93. The article of claim 85 wherein at least a portion of said polynucleotides are at least ten Kb long.

94. The article of claim 85 wherein at least a portion of said polynucleotides are circularized.

95. The article of claim 85 wherein the polynucleotides comprise a polynucleotide sequence flanked by adapter sequences to facilitate manipulation of the polynucleotide sequence.

96. The article of claim 95 wherein the adapter sequences facilitate one or more of insertion into a vector, immobilization, and identification of a function of the sequence.

97. The article of claim 85 wherein said mixture of polynucleotides comprises one or more sequences selected from the group consisting of mammalian sequences, yeast sequences, prokaryotic sequences, plant sequences, D. melanogaster sequences, C. elegans sequences, and Xenopus sequences.

98. The article of claim 85 wherein the mixture of different, retrievable polynucleotide constructs are independently retrievable.

99. The article of claim 85 comprising plural polynucleotide reservoirs containing plural different polynucleotides, the polynucleotides in different reservoirs comprising an identical said pair of primer sequences.

100-102. (canceled)

103. The article of claim 85 wherein a polynucleotide reservoir contains different polynucleotides comprising plural nested pairs of primer sequences, each of said plural nested pairs permitting amplification of a selected group of polynucleotides in said reservoir or of individual ones of said different polynucleotides therein.

104. The article of claim 85 comprising 10² different polynucleotides.

105. The article of claim 85 comprising 10³ different polynucleotides.

106. The article of claim 85 comprising 10⁴ different polynucleotides.

107. The article of claim 85 comprising 10⁵ different polynucleotides.

108. The article of claim 85 comprising 10⁶ different polynucleotides.

109. An article of manufacture comprising a package containing a multiplicity of different, retrievable polynucleotides, the article comprising:
a polynucleotide reservoir which contains a mixture of different polynucleotides at least some of which comprise plural nested pairs of primer sequences, each of said plural nested pairs permitting amplification of a selected group of polynucleotides in said reservoir or of individual ones of said different polynucleotides therein; and
plural primer reservoirs each of which contains a pair of oligonucleotide primers complementary to a pair of primer sequences of a polynucleotide in said construct reservoir.

110-119. (canceled)

120. A method of obtaining a polynucleotide of choice comprising the steps of:
providing plural construct reservoirs containing mixtures of identified synthesized polynucleotides comprising plural nested pairs of primer sequences which permit amplification of selected ones of said polynucleotides from a said reservoir, the combination of primer pairs of a polynucleotide in a said reservoir being different from other pairs of primer sequence of other polynucleotides in said reservoir;
providing plural primer reservoirs each of which contains a pair of oligonucleotide primers complementary to a pair of primer sequences of a polynucleotide in a said construct reservoirs;
conducting a first amplification procedure in a first amplification mixture comprising an aliquot of a said mixture of polynucleotides retrieved from a selected said construct reservoir and a pair of primers complementary to an outer nested pair of primer sequences retrieved from one or more primer reservoirs; and
conducting a second amplification procedure in a second amplification mixture comprising an aliquot of amplicons retrieved from said first amplification mixture and a pair of primers complementary to an inner nested pair of primer sequences retrieved from one or more primer reservoirs.

121-124. (canceled)
